(19) United States
(12) Patent Application Publication
(10) Pub. No.: US 2023/0372263 A1
(43) Pub. Date: Nov. 23, 2023

(54) METHODS AND COMPOSITIONS FOR TREATING AMYOTROPHIC LATERAL SCLEROSIS

(71) Applicant: Amylyx Pharmaceuticals, Inc., Cambridge, MA (US)

(72) Inventors: Joshua Cohen, Canton, MA (US); Justin Klee, Cambridge, MA (US)

(21) Appl. No.: 17/746,458

(22) Filed: May 17, 2022

(51) Int. Cl. A61K 31/05; A61K 31/215; A61K 9/00; A61J 15/00; A61P 25/00

(52) U.S. Cl. CPC A61K 31/05; A61K 31/215; A61K 9/0073; A61J 15/0011; A61P 25/00

(57) ABSTRACT

Provided herein are methods and compositions for treating a neurodegenerative disease (e.g., ALS). The methods can include administering to the subject a bile acid or a pharmaceutically acceptable salt thereof and a phenylbutyrate compound.

### TECHNICAL FIELD

The present disclosure generally relates to compositions and methods for treating Amyotrophic lateral sclerosis.

### BACKGROUND

Amyotrophic lateral sclerosis (ALS) is the most prevalent progressive motor neuron disease. ALS causes the progressive degeneration of motor neurons, resulting in rapidly progressing muscle weakness and atrophy that eventually leads to partial or total paralysis. Median survival from symptom onset is 2 to 3 years, with respiratory failure being the predominant cause of death. ALS treatment currently centers on symptom management. Only two FDA-approved medications for ALS, riluzole and edaravone, are presently available. Accordingly, there is a need for improved therapies for treating ALS.

### SUMMARY

Provided herein are methods of treating at least one symptom of ALS in a subject, the method comprising: (a) administering to the subject one or more doses of a composition comprising about 1 gram of Taurursodiol (TURSO) and about 3 grams of sodium phenylbutyrate, (b) determining that the subject has (1) a Cₘₐₓ for sodium phenylbutyrate of about 3 to about 425 μg/mL, and/or (2) a Cₘₐₓ for phenylacetate of about 5 to about 50 μg/mL, and (c) administering to the subject an additional dose of the composition. In some embodiments, the methods further comprise determining that the subject has a Cₘₐₓ for sodium phenylbutyrate of about 90 to about 170 μg/mL. In some embodiments, the methods further comprise determining that the subject has a Cₘₐₓ for sodium phenylbutyrate of about 110 to about 150 μg/mL. In some embodiments, the methods further comprise determining that the subject has a Cₘₐₓ for phenylacetate of about 10 to about 45 μg/mL. In some embodiments, the Cₘₐₓ is steady-state Cₘₐₓ.

Also provided herein are methods of treating at least one symptom of ALS in a subject, the method comprising: (a) administering to the subject one or more doses of a composition comprising about 1 gram of TURSO and about 3 grams of sodium phenylbutyrate, (b) determining that the subject has (1) an AUC₀₋ₗₐₛₜ for sodium phenylbutyrate of about 20 to about 550 μg*h/mL, and/or (2) an AUC₀₋ₗₐₛₜ for phenylacetate of about 20 to about 160 μg*h/mL, and (c) administering to the subject an additional dose of the composition. In some embodiments, the methods further comprise determining that the subject has an AUC₀₋ₗₐₛₜ for sodium phenylbutyrate of about 140 to about 300 μg*h/mL. In some embodiments, the methods further comprise determining that the subject has an AUC₀₋ₗₐₛₜ for phenylacetate of about 40 to about 80 μg*h/mL.

Also provided herein are methods of treating at least one symptom of ALS in a subject, the method comprising: (a) administering to the subject one or more doses of a composition comprising about 1 gram of TURSO and about 3 grams of sodium phenylbutyrate, (b) determining that the subject has (1) an AUC_{0-∞} for sodium phenylbutyrate of about 25 to about 545 μg*h/mL, and/or (2) an AUC_{0-∞} for phenylacetate of about 21 to about 155 μg*h/mL, and (c) administering to the subject an additional dose of the composition. In some embodiments, the methods further comprise determining that the subject has an AUC_{0-∞} for sodium phenylbutyrate of about 140 to about 300 μg*h/mL. In some embodiments, the methods further comprise determining that the subject has an AUC_{0-∞} for phenylacetate of about 40 to about 80 μg*h/mL.

In some embodiments, in any of the methods described above, step (a) comprises administering the composition once a day or twice a day for about 1 day to about 40 weeks. In some embodiments, in any of the methods described above, step (a) comprises administering the composition once a day or twice a day for about 10 weeks to about 26 weeks. In some embodiments, in any of the methods described above, step (a) comprises administering the composition twice a day for about 9 weeks to about 21 weeks. In some embodiments, in any of the methods described above, step (a) comprises administering the composition once a day for about 3 weeks followed by twice a day for about 9 weeks to about 21 weeks. In some embodiments, in any of the methods described above, step (b) comprises obtaining a blood sample from the subject about one hour after the last dose of the composition. In some embodiments, in any of the methods described above, step (b) comprises obtaining a blood sample from the subject about four hours after the last dose of the composition.

Also provided herein are methods of treating at least one symptom of ALS in a subject, the method comprising. (a) administering to the subject one or more doses of a composition comprising about 1 gram of TURSO and about 3 grams of sodium phenylbutyrate; (b) determining a plasma concentration of one or more bile acids selected from TURSO, UDCA, or GUDCA in the subject; and (c) administering to the subject an additional dose of the composition. In some embodiments, the plasma concentration is steady-state plasma concentration. In some embodiments, step (a) comprises administering the composition once a day or twice a day for about 1 day to about 40 weeks. In some embodiments, step (a) comprises administering the composition once a day or twice a day for about 10 weeks to about 26 weeks. In some embodiments, step (a) comprises administering the composition twice a day for about 9 weeks to about 21 weeks. In some embodiments, step (a) comprises administering the composition once a day for about 3 weeks followed by twice a day for about 9 weeks to about 21 weeks. In some embodiments, step (b) comprises determining the plasma concentration about one hour after the last dose of the composition. In some embodiments, step (b) comprises determining the plasma concentration about four hours after the last dose of the composition.

In some embodiments, step (b) comprises determining the plasma concentration of TURSO in the subject about one hour after the last dose of the composition, wherein the plasma concentration of TURSO is about 20 to about 2570 ng/mL. In some embodiments, wherein the plasma concentration of TURSO is about 20 to about 1045 ng/mL. In some embodiments, wherein the plasma concentration of TURSO is about 88 to about 540 ng/mL. In some embodiments, wherein step (b) comprises determining the plasma concentration of TURSO in the subject about four hours after the last dose of the composition, wherein the plasma concentration of TURSO is about 20 to about 3250 ng/mL. In some embodiments, wherein the steady-state plasma concentration of TURSO is about 20 to about 1125 ng/mL. In some embodiments, wherein the steady-state plasma concentration of TURSO is about 155 to about 785 ng/mL.

In some embodiments, step (b) comprises determining the plasma concentration of UDCA in the subject about one hour after the last dose of the composition, wherein the plasma concentration of UDCA is about 20 to about 6020 ng/mL. In some embodiments, wherein the plasma concentration of UDCA is about 20 to about 1955 ng/mL. In some embodiments, the plasma concentration of UDCA is about 285 to about 1125 ng/mL. In some embodiments, step (b) comprises determining the plasma concentration of UDCA in the subject about four hours after the last dose of the composition, wherein the plasma concentration of UDCA is about 20 to about 7340 ng/mL. In some embodiments, the plasma concentration of UDCA is about 20 to about 2550 ng/mL. In some embodiments, the plasma concentration of UDCA is about 305 to about 1395 ng/mL.

In some embodiments, step (b) comprises determining the plasma concentration of GUDCA in the subject about one hour after the last dose of the composition, wherein the plasma concentration of GUDCA is about 20 to about 4600 ng/mL. In some embodiments, the plasma concentration of GUDCA is about 65 to about 2085 ng/mL. In some embodiments, the plasma concentration of GUDCA is about 340 to about 1635 ng/mL. In some embodiments, step (b) comprises determining the plasma concentration of GUDCA in the subject about four hours after the last dose of the composition, wherein the plasma concentration of GUDCA is about 20 to about 5290 ng/mL. In some embodiments, the plasma concentration of GUDCA is about 320 to about 2315 ng/mL. In some embodiments, the plasma concentration of GUDCA is about 530 to about 1915 ng/mL.

In some embodiments, the method further comprises, prior to step (a), determining a baseline plasma concentration of the bile acid in the subject. In some embodiments, the method comprises determining a baseline plasma concentration of TURSO in the subject, and wherein the baseline plasma concentration of TURSO is about 20 to about 577 ng/mL. In some embodiments, the baseline plasma concentration of TURSO is about 20 to about 125 ng/mL.

In some embodiments, the method comprises determining a baseline plasma concentration of UDCA in the subject, and wherein the baseline plasma concentration of UDCA is about 20 to about 5970 ng/mL. In some embodiments, the baseline plasma concentration of UDCA is about 20 to about 825 ng/mL. In some embodiments, the baseline plasma concentration of UDCA is about 20 to about 53 ng/mL.

In some embodiments, the method comprises determining a baseline plasma concentration of GUDCA in the subject, and wherein the baseline plasma concentration of GUDCA is about 20 to about 4540 ng/mL. In some embodiments, the baseline plasma concentration of GUDCA is about 20 to about 755 ng/mL. In some embodiments, the baseline plasma concentration of GUDCA is about 25 to about 180 ng/mL.

In some embodiments of any of the above-mentioned methods, step (a) comprises administering a dose of the composition more than two hours after the subject has consumed food, or more than one hour before the subject consumes food.

Also provided herein are methods of increasing the plasma concentration of a bile acid in a subject, the method comprising administering to the subject one or more doses of a composition comprising about 1 gram of TURSO and about 3 grams of sodium phenylbutyrate, wherein the bile acid is selected from TURSO, UDCA or GUDCA, wherein where the bile acid is TURSO, the plasma concentration is about 20 to about 3250 ng/mL, wherein where the bile acid is UDCA, the plasma concentration is about 20 to about 7340 ng/mL, and wherein where the bile acid is GUDCA, the plasma concentration is about 20 to about 5290 ng/mL. In some embodiments, the plasma concentration is steady-state plasma concentration. In some embodiments, the method comprises administering the composition once a day or twice a day for about 1 day to about 40 weeks. In some embodiments, the method comprises administering the composition once a day or twice a day for about 10 weeks to about 26 weeks. In some embodiments, the method comprises administering the composition twice a day for about 9 weeks to about 21 weeks. In some embodiments, the method comprises administering the composition once a day for about 3 weeks followed by twice a day for about 9 weeks to about 21 weeks. In some embodiments, the method comprises determining the plasma concentration of the bile acid about one hour after the last dose of the composition. In some embodiments, the method comprises determining the plasma concentration of the bile acid about four hours after the last dose of the composition.

In some embodiments of any of the above-mentioned methods, the composition is administered orally. In some embodiments of any of the above-mentioned methods, the composition is administered through a feeding tube. In some embodiments of any of the above-mentioned methods, the composition is administered by bolus injection. In some embodiments of any of the above-mentioned methods, the composition is a powder formulation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a flowchart of the study sequence followed in Example 1.

FIG. 2 is a graph showing the geometric mean (×/÷Geometric SD) plasma concentrations for PB following single oral doses of AMX0035 in the fasted and fed states on a Log₁₀/Linear Scale.

FIG. 3 is a graph showing the geometric mean (×/÷Geometric SD) plasma concentrations for PAA following single oral doses of AMX0035 in the fasted and fed states on a Log₁₀/Linear Scale.

FIG. 4 is a graph showing the geometric mean (×/÷Geometric SD) plasma concentrations for TURSO following single oral doses of AMX0035 in the fasted and fed states on a Log₁₀/Linear Scale.

FIG. 5 is a graph showing the geometric mean (×/÷Geometric SD) plasma concentrations for UDCA following single oral doses of AMX0035 in the fasted and fed states on a Log₁₀/Linear Scale.

FIG. 6 is a graph showing the geometric mean (×/÷Geometric SD) plasma concentrations for GUDCA following single oral doses of AMX0035 in the fasted and fed states on a Log₁₀/Linear Scale.

FIG. 7 are TUDCA Boxplots of Sex-by-Pooled-Visit Groups.

FIG. 8 are GUDCA Boxplots of Sex-by-Pooled-Visit Groups.

FIG. 9 are UDCA Boxplots of Age-Categories-by-Pooled-Visit Groups.

FIG. 10 are GUDCA Boxplots of Age-Categories-by-Pooled-Visit Groups.

FIG. 11 are TUDCA Boxplots of Antibiotic-Use-by-Pooled-Visit Groups.

FIG. 12 are UDCA Boxplots of Antibiotic-Use-by-Pooled-Visit Groups.

FIG. 13 are GUDCA Boxplots of Antibiotic-Use-by-Pooled-Visit Groups.

FIG. 14 are TUDCA Boxplots of Glomerular-Filtration-Rate-Category-by-Pooled-Visit Groups.

FIG. 15 are UDCA Boxplots of Glomerular-Filtration-Rate-Category-by-Pooled-Visit Groups.

FIG. 16 are GUDCA Boxplots of Glomerular-Filtration-Rate-Category-by-Pooled-Visit Groups.

FIG. 17 is a diagram showing pathway visualization of changes in CC based on ANOVA results (Week 12).

FIG. 18 is a diagram showing pathway visualization of changes in CC based on ANOVA results (Week 24).

### DETAILED DESCRIPTION

In one aspect, provided herein are methods of treating at least one symptom of ALS in a subject, the methods include (a) administering to the subject one or more doses of a composition comprising about 1 gram of Taurursodiol (TURSO) and about 3 grams of sodium phenylbutyrate, (b) determining that the subject has (1) a Cₘₐₓ for sodium phenylbutyrate of about 3 to about 425 μg/mL, and/or (2) a Cₘₐₓ for phenylacetate of about 5 to about 50 μg/mL, and (c) administering to the subject an additional dose of the composition. In another aspect, provided herein are methods of treating at least one symptom of ALS in a subject, the methods include: (a) administering to the subject one or more doses of a composition comprising about 1 gram of TURSO and about 3 grams of sodium phenylbutyrate, (b) determining that the subject has (1) an AUC₀₋ₗₐₛₜ for sodium phenylbutyrate of about 20 to about 550 μg*h/mL, and/or (2) an AUC₀₋ₗₐₛₜ for phenylacetate of about 20 to about 160 μg*h/mL, and (c) administering to the subject an additional dose of the composition. In another aspect, provided herein are methods of treating at least one symptom of ALS in a subject, the methods include: (a) administering to the subject one or more doses of a composition comprising about 1 gram of TURSO and about 3 grams of sodium phenylbutyrate, (b) determining that the subject has (1) an AUC_{0-∞} for sodium phenylbutyrate of about 25 to about 545 μg*h/mL, and/or (2) an AUC_{0-∞} for phenylacetate of about 21 to about 155 μg*h/mL, and (c) administering to the subject an additional dose of the composition.

Further aspects of the present disclosure involve methods of treating at least one symptom of ALS in a subject, the methods include: (a) administering to the subject one or more doses of a composition comprising about 1 gram of TURSO and about 3 grams of sodium phenylbutyrate; (b) determining a plasma concentration of one or more bile acids selected from TURSO, UDCA, or GUDCA in the subject; (c) administering to the subject an additional dose of the composition.

An additional aspect of the present disclosure involves methods of increasing the plasma concentration of a bile acid in a subject, the methods include administering to the subject one or more doses of a composition comprising about 3 grams of sodium phenylbutyrate and about 1 gram of TURSO, wherein the bile acid is selected from TURSO, UDCA and GUDCA, wherein where the bile acid is TURSO, the plasma concentration is about 20 to about 3250 ng/mL, wherein where the bile acid is UDCA, the plasma concentration is about 20 to about 7340 ng/mL, and wherein where the bile acid is GUDCA, the plasma concentration is about 20 to about 5290 ng/mL.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Throughout the specification it is noted that μg-h/mL, μg*h/mL, and μg·h/ml are used interchangeably. Likewise, ng-h/mL, ng*h/mL, and ng·h/ml are also used interchangeably.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

I. Amyotrophic Lateral Sclerosis (ALS)

The terms “amyotrophic lateral sclerosis” and “ALS” are used interchangeably herein, and include all of the classifications of ALS known in the art, including, but not limited to classical ALS (e.g., ALS that affects both lower and upper motor neurons), Primary Lateral Sclerosis (PLS, e.g., those that affect only the upper motor neurons), Progressive Bulbar Palsy (PBP or Bulbar Onset, a version of ALS that typically begins with difficulties swallowing, chewing and speaking) and Progressive Muscular Atrophy (PMA, typically affecting only the lower motor neurons). The terms include sporadic and familial (hereditary) ALS, ALS at any rate of progression (e.g., rapid, non-slow or slow progression) and ALS at any stage (e.g., prior to onset, at onset and late stages of ALS).

The subjects in the methods described herein may exhibit one or more symptoms associated with ALS, or have been diagnosed with ALS. In some embodiments, the subjects may be suspected as having ALS, and/or at risk for developing ALS.

The subjects in the methods described herein may exhibit one or more symptoms associated with benign fasciculation syndrome (BFS) or cramp-fasciculation syndrome (CFS).

Some embodiments of any of the methods described herein can further include determining that a subject has or is at risk for developing ALS, diagnosing a subject as having or at risk for developing ALS, or selecting a subject having or at risk for developing ALS. Likewise, some embodiments of any of the methods described herein can further include determining that a subject has or is at risk for developing benign fasciculation syndrome or cramp fasciculation syndrome, diagnosing a subject as having or at risk for developing BFS or CFS, or selecting a subject having or at risk for developing BFS or CFS.

In some embodiments of any of the methods described herein, the subject has shown one or more symptoms of ALS for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of ALS for about 36 months or less (e.g., about 35, 34, 33, 32, 31, 30, 29, 28, 27, 26, or 25 months or less).

The order and type of ALS symptoms displayed by a subject may depend on which motor neurons in the body are damaged first, and consequently which muscles in the body are damaged first. For example, bulbar onset, limb onset, or respiratory onset ALS may present with similar or different symptoms. In general, ALS symptoms may include muscle weakness or atrophy (e.g., affecting upper body, lower body, and/or speech), muscle fasciculation (twitching), cramping, or stiffness of affected muscles. Early symptoms of ALS may include those of the arms or legs, difficulty in speaking clearly or swallowing (e.g., in bulbar onset ALS). Other symptoms include loss of tongue mobility, respiratory difficulties, difficulty breathing or abnormal pulmonary function, difficulty chewing, and/or difficulty walking (e.g., resulting in stumbling). Subjects may have respiratory muscle weakness as the initial manifestation of ALS symptoms. Such subjects may have very poor prognosis and in some instances have a median survival time of about two months from diagnosis. In some subjects, the time of onset of respiratory muscle weakness can be used as a prognostic factor.

ALS symptoms can also be classified by the part of the neuronal system that is degenerated, namely, upper motor neurons or lower motor neurons. Lower motor neuron degeneration manifests, for instance, as weakness or wasting in one or more of the bulbar, cervical, thoracic, and/or lumbosacral regions. Upper motor neuron degeneration can include increased tendon reflexes, spasticity, pseudo bulbar features, Hoffmann reflex, extensor plantar response, and exaggerated reflexes (hyperreflexia) including an overactive gag reflex. Progression of neuronal degeneration or muscle weakness is a hallmark of the disease. Accordingly, some embodiments of the present disclosure provide a method of ameliorating at least one symptom of lower motor neuron degeneration, at least one symptom of upper motor neuron degeneration, or at least one symptom from each of lower motor neuron degeneration and upper motor neuron degeneration. In some embodiments of any of the methods described herein, symptom onset can be determined based on information from subject and/or subject's family members. In some embodiments, the median time from symptom onset to diagnosis is about 12 months.

In some instances, the subject has been diagnosed with ALS. For example, the subject may have been diagnosed with ALS for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with ALS for 1 week or less, or on the same day that the presently disclosed treatments are administered. The subject may have been diagnosed with ALS for more than about 24 months (e.g., more than about 28, 32, 36, 40, 44, 48, 52, 56, 60, 64, 68, 72, 76, or 80 months). Methods of diagnosing ALS are known in the art. For example, the subject can be diagnosed based on clinical history, family history, physical or neurological examinations (e.g., signs of lower motor neuron or upper motor neuron degeneration). The subject can be confirmed or identified, e.g. by a healthcare professional, as having ALS. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner (e.g., a general practitioner).

In some embodiments, the subject fulfills the El Escorial criteria for probable or definite ALS, i.e. the subject presents.
1. Signs of lower motor neuron (LMN) degeneration by clinical, electrophysiological or neuropathologic examination;
2. Signs of upper motor neuron (UMN) degeneration by clinical examination; and
3. Progressive spread of signs within a region or to other regions, together with the absence of:
Electrophysiological evidence of other disease processes that might explain the signs of LMN and/or UMN degenerations; and
Neuroimaging evidence of other disease processes that might explain the observed clinical and electrophysiological signs.

Under the El Escorial criteria, signs of LMN and UMN degeneration in four regions are evaluated, including brainstem, cervical, thoracic, and lumbrasacral spinal cord of the central nervous system. The subject may be determined to be one of the following categories:
A. Clinically Definite ALS, defined on clinical evidence alone by the presence of UMN, as well as LMN signs, in three regions.
B. Clinically Probable ALS, defined on clinical evidence alone by UMN and LMN signs in at least two regions with some UMN signs necessarily rostral to (above) the LMN signs.
C. Clinically Probable ALS—Laboratory-supported, defined when clinical signs of UMN and LMN dysfunction are in only one region, or when UMN signs alone are present in one region, and LMN signs defined by EMG criteria are present in at least two limbs, with proper application of neuroimaging and clinical laboratory protocols to exclude other causes.
D. Clinically Possible ALS, defined when clinical signs of UMN and LMN dysfunction are found together in only one region or UMN signs are found alone in two or more regions; or LMN signs are found rostral to UMN signs and the diagnosis of Clinically Probable-Laboratory-supported.

In some embodiments, the subject has clinically definite ALS (e.g., based on the El Escorial criteria).

The subject can be evaluated and/or diagnosed using the Revised Amyotrophic Lateral Sclerosis Functional Rating Scale (ALSFRS-R). The ALSFRS-R is an ordinal rating scale (ratings 0-4) used to determine subjects' assessment of their capability and independence in 12 functional activities relevant in ALS. ALSFRS-R scores calculated at diagnosis can be compared to scores throughout time to determine the speed of progression. Change in ALSFRS-R scores can be correlated with change in strength over time, and can be associated with quality of life measures and predicted survival. ALSFRS-R demonstrates a linear mean slope and can be used as a prognostic indicator (See e.g., Berry et al. Amyotroph Lateral Scler Frontotemporal Degener 15:1-8, 2014; Traynor et al., Neurology 63:1933-1935, 2004; Simon et al., Ann Neurol 76:643-657, 2014; and Moore et al. Amyotroph Lateral Scler Other Motor Neuron Disord 4:42, 2003).

In the ALSFRS-R, functions mediated by cervical, trunk, lumbosacral, and respiratory muscles are each assessed by 3 items. Each item is scored from 0-4, with 4 reflecting no involvement by the disease and 0 reflecting maximal involvement. The item scores are added to give a total. Total scores reflect the impact of ALS, with the following exemplary categorization:
>40 (minimal to mild); 39-30 (mild to moderate), <30 (moderate to severe); <20 (advanced disease).

For example, a subject can have an ALSFRS-R score (e.g., a baseline ALSFRS-R score) of 40 or more (e.g., at least 41, 42, 43, 44, 45, 46, 47, or 48), between 30 and 39, inclusive (e.g., 31, 32, 33, 34, 35, 36, 37, or 38), or 30 or less (e.g., 21, 22, 23, 24, 25, 26, 27, 28, or 29). In some embodiments of any of the methods described herein, the subject has an ALSFRS-R score (e.g., a baseline ALSFRS-R score) of 40 or less (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10 or less). In some embodiments, the subject has an ALSFRS-R score (e.g., a baseline ALSFRS-R score) of 20 or less (e.g., 19, 18, 17, 16, 15, 14, 13, 12, 11, 10, 9, 8, 7, 6, 5 or less).

As ALS is a progressive disease, all patients generally will progress over time. However, a large degree of inter-subject variability exists in the rate of progression, as some subjects die or require respiratory support within months while others have relatively prolonged survival. The subjects described herein may have rapid progression ALS or slow progression ALS. The rate of functional decline in a subject with ALS can be measured by the change in ALSFRS-R score per month. For example, the score can decrease by about 1.02 (±2.3) points per month.

One predictor of patient progression is the patient's previous rate of disease progression (ΔFS), which can be calculated as: ΔFS=(48−ALSFRS-R score at the time of evaluation)/duration from onset to time of evaluation (month). The ΔFS score represents the number of ALSFRS-R points lost per month since symptom onset, and can be a significant predictor of progression and/or survival in subjects with ALS (See e.g., Labra et al. J Neurol Neurosurg Psychiatry 87:628-632, 2016 and Kimura et al. Neurology 66:265-267, 2006). The subject may have a disease progression rate (ΔFS) of about 0.50 or less (e.g., about 0.45, 0.40, 0.35, 0.30, 0.25, 0.20, 0.15, or 0.10 or less); between about 0.50 and about 1.20 inclusive (e.g., about 0.55, 0.60, 0.65, 0.70, 0.75, 0.80, 0.85, 0.90, 0.95, 1.00, 1.05, 1.10, or 1.15); or about 1.20 or greater (e.g., about 1.25, 1.30, 1.35, 1.40, 1.45, 1.50, 1.55, 1.60, 1.75, 1.80, 1.85, 1.90, 1.95, or 2.00 or greater). In some embodiments of any of the methods described herein, the subject can have an ALS disease progression rate (ΔFS) of about 0.50 or greater (e.g., about 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.75, 1.80, 1.85, 1.90, 1.95, or 2.00 or greater). However, it should be noted that the ΔFS score is a predictor of patient progression, and may under or overestimate a patient's progression once under evaluation.

In some embodiments, since initial evaluation, the subject has lost on average about 0.8 to about 2 (e.g., about 0.9, 1.0, 1.1, 1.2, 1.3, 1.4, 1.5, 1.6, 1.7, 1.8, or 1.9) ALSFRS-R points per month over 3-12 months. In some embodiments, the subject has lost on average more than about 1.2 ALSFRS-R points per month over 3-12 months since initial evaluation. The subject may have had a decline of at least 3 points (e.g., at least 4, 6, 8, 10, 12, 14, 16, 20, 24, 28, or 32 points) in ALSFRS-R score over 3-12 months since initial evaluation. In some embodiments, the subject has lost on average about 0.8 to about 2 (e.g., about 0.9, 1.0, 1.1, 1.2, 1.3, 1.4, 1.5, 1.6, 1.7, 1.8, or 1.9) ALSFRS-R points per month over the previous 3-12 months. In some embodiments, the subject has lost on average more than about 1.2 (e.g., more than about 1.5, 1.8, 2.0, 2.5, or 3) ALSFRS-R points per month over the previous 3-12 months.

In some embodiments of any of the methods described herein, the presence or level of a marker in a sample obtained from the subject may be used for ALS diagnosis or prognosis, or to track disease activity and treatment responses. Suitable samples include, for example, cells, tissues, or body fluids (e.g. blood, urine, or cerebral spinal fluid (CSF) samples). For instance, levels of phosphorylated neurofilament heavy subunit (pNF-H) or neurofilament light chain (NfL) in the CSF and/or blood can be used as a biomarker for ALS diagnosis, prognosis, or to track disease activity or treatment outcomes. pNF-H is a main component of the neuronal cytoskeleton and is released into the CSF and the bloodstream with neuronal damage. Levels of pNF-H may correlate with the level of axonal loss and/or burden of motor neuron dysfunction (See, e.g., De Schaepdryver et al. Journal of Neurology, Neurosurgery & Psychiatry 89:367-373, 2018).

The concentration of pNF-H in the CSF and/or blood of a subject with ALS may significantly increase in the early disease stage. Higher levels of pNF-H in the plasma, serum and/or CSF may be associated with faster ALS progression (e.g., faster decline in ALSFRS-R), and/or shorter survival. pNF-H concentration in plasma may be higher in ALS subjects with bulbar onset than those with spinal onset. In some cases, an imbalance between the relative expression levels of the neurofilament heavy and light chain subunits can be used for ALS diagnosis, prognosis, or tracking disease progression.

Methods of detecting pNF-H and NfL (for example, in the cerebrospinal fluid, plasma, or serum) are known in the art and include but are not limited to, ELISA and Simoa assays (See e.g., Shaw et al. Biochemical and Biophysical Research Communications 336:1268-1277, 2005; Ganesalingam et al. Amyotroph Lateral Scler Frontotemporal Degener 14(2):146-9, 2013; De Schaepdryver et al. Annals of Clinical and Translational Neurology 6(10): 1971-1979, 2019; Wilke et al. Clin Chem Lab Med 57(10):1556-1564, 2019; Poesen et al. Front Neurol 9:1167, 2018; Pawlitzki et al. Front. Neurol. 9:1037, 2018; Gille et al. Neuropathol Appl Neurobiol 45(3):291-304, 2019). Commercialized pNF-H detection assays can also be used, such as those developed by EnCor Biotechnology, BioVendor, and Millipore-EMD. Commercial NfL assay kits based on the Simoa technology, such as those produced by Quanterix can also be used (See, e.g., Thouvenot et al. European Journal of Neurology 27:251-257, 2020). Factors affecting pNF-H and NfL levels or their detection in serum or plasma in relation to disease course may differ from those in CSF. The levels of neurofilament (e.g. pNF-H and/or NfL) in the CSF and serum may be correlated (See, e.g., Wilke et al. Clin Chem Lab Med 57(10):1556-1564, 2019).

Subjects described herein may have a CSF or blood pNF-H level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, the serum pNF-H level can be about 70 to about 1200 pg/mL (e.g., about 70 to about 1000, about 70 to about 800, about 80 to about 600, or about 90 to about 400 pg/mL). In some embodiments, the CSF pNF-H level can be about 1000 to about 5000 pg/mL (e.g., about 1500 to about 4000, or about 2000 to about 3000 pg/mL).

The subjects may have a CSF or blood level of NfL of about 50 pg/mL or higher (e.g., about 60, 70, 80, 90, 100, 110, 120, 130, 140, 150, 160, 170, 180, 190, 200, 210, 220, 230, 240, or 250 pg/mL or higher). In some embodiments, the serum NfL level can be about 50 to about 300 pg/mL (e.g., about 50 to about 280, about 50 to about 250, about 50 to about 200, about 50 to about 150, about 50 to about 100, about 100 to about 300, about 100 to about 250, about 100 to about 200, about 100 to about 150, about 150 to about 300, about 150 to about 250, about 150 to about 200, about 200 to about 300, about 200 to about 250, or about 250 to about 300 pg/mL). In some embodiments, the CSF NfL level can be about 2000 to about 40,000 pg/mL (e.g., about 2000 to about 35,000, about 2000 to about 30,000, about 2000 to about 25,000, about 2000 to about 20,000, about 2000 to about 15,000, about 2000 to about 10,000, about 2000 to about 8000, about 2000 to about 6000, about 2000 to about 4000, about 4000 to about 40.000, about 4000 to about 35.000, about 4000 to about 30,000, about 4000 to about 25,000, about 4000 to about 20.000, about 4000 to about 15,000, about 4000 to about 10,000, about 4000 to about 8000, about 4000 to about 6000, about 6000 to about 40,000, about 6000 to about 35,000, about 6000 to about 30,000, about 6000 to about 25,000, about 6000 to about 20,000, about 6000 to about 15,000, about 6000 to about 10,000, about 6000 to about 8000, about 8000 to about 40,000, about 8000 to about 35,000, about 8000 to about 30,000, about 8000 to about 25,000, about 8000 to about 20,000, about 8000 to about 15,000, about 8000 to about 10,000, about 10,000 to about 40,000, about 10,000 to about 35,000, about 10,000 to about 30.000, about 10.000 to about 25,000, about 10.000 to about 20,000, about 10,000 to about 15,000, about 15,000 to about 40,000, about 15,000 to about 35,000, about 15,000 to about 30,000, about 15,000 to about 25,000, about 15,000 to about 20,000, about 20,000 to about 40,000, about 20,000 to about 35,000, about 20,000 to about 30,000, about 20,000 to about 25,000, about 25,000 to about 40,000, about 25,000 to about 35,000, about 25,000 to about 30,000, about 30,000 to about 40,000, about 30,000 to about 35,000, or about 35,000 to about 40,000 pg/mL).

Additional biomarkers useful for ALS diagnosis, prognosis, and disease progression monitoring are contemplated herein, including but are not limited to, CSF levels of S100-β, cystatin C, and chitotriosidase (CHIT)(See e.g., Chen et al. BMC Neurol 16:173, 2016). Serum levels of uric acid can be used as a biomarker for prognosing ALS (See e.g., Atassi et al. Neurology 83(19):1719-1725, 2014). Akt phosphorylation can also be used as a biomarker for prognosing ALS (See e.g., WO2012/160563). Urine levels of p75ECD and ketones can be used as a biomarker for ALS diagnosis (See e.g., Shepheard et al. Neurology 88:1137-1143, 2017). Serum and urine levels of creatinine can also be used as a biomarker. Other useful blood, CSF, neurophysiological, and neuroradiological biomarkers for ALS are described in e.g., Turner et al. Lancet Neurol 8:94-109, 2009. Any of the markers described herein can be used for diagnosing a subject as having ALS, or determining that a subject is at risk for developing ALS.

A subject may also be identified as having ALS, or at risk for developing ALS, based on genetic analysis. Genetic variants associated with ALS are known in the art (See, e.g., Taylor et al. Nature 539:197-206, 2016; Brown and Al-Chalabi N Engl J Med 377:162-72, 2017; and http://alsod.iop.kcl.ac.uk). Subjects described herein can carry mutations in one or more genes associated with familial and/or sporadic ALS. Exemplary genes associated with ALS include but are not limited to: ANG, TARDBP, VCP, VAPB, SQSTM1, DCTN1, FUS, UNC13A, ATXN2, HNRNPA1, CHCHD10, MOBP, C21ORF2, NEK1, TUBA4A, TBK1, MATR3, PFN1, UBQLN2, TAF15, OPTN, TDP-43, and DAO. Additional description of genes associated with ALS can be found at Therrien et al. Curr Neurol Neurosci Rep 16:59-71, 2016: Peters et al. J Clin Invest 125:2548, 2015, and Pottier et al. J Neurochem, 138:Suppl 1:32-53, 2016. Genetic variants associated with ALS can affect the ALS progression rate in a subject, the pharmacokinetics of the administered compounds in a subject, and/or the efficacy of the administered compounds for a subject.

The subjects may have a mutation in the gene encoding CuZn-Superoxide Dismutase (SOD1). Mutation causes the SOD1 protein to be more prone to aggregation, resulting in the deposition of cellular inclusions that contain misfolded SOD1 aggregates (See e.g., Andersen et al., Nature Reviews Neurology 7:603-615, 2011). Over 100 different mutations in SOD1 have been linked to inherited ALS, many of which result in a single amino acid substitution in the protein. In some embodiments, the SOD1 mutation is A4V (i.e., a substitution of valine for alanine at position 4). SOD1 mutations are further described in, e.g., Rosen et al. Hum. Mol. Genet. 3, 981-987, 1994 and Rosen et al. Nature 362:59-62, 1993. In some embodiments, the subject has a mutation in the C9ORF72 gene. Repeat expansions in the C9ORF72 gene are a frequent cause of ALS, with both loss of function of C9ORF72 and gain of toxic function of the repeats being implicated in ALS (See e.g., Balendra and Isaacs, Nature Reviews Neurology 14:544-558, 2018). The methods described herein can include, prior to administration of a bile acid and a phenylbutyrate compound, detecting a SOD1 mutations and/or a C9ORF72 mutation in the subject. Methods for screening for mutations are well known in the art. Suitable methods include, but are not limited to, genetic sequencing. See, e.g., Hou et al. Scientific Reports 6:32478, 2016; and Vajda et al. Neurology 88:1-9, 2017.

Skilled practitioners will appreciate that certain factors can affect the bioavailability and metabolism of the administered compounds for a subject, and can make adjustments accordingly. These include but are not limited to liver function (e.g. levels of liver enzymes), renal function, and gallbladder function (e.g., ion absorption and secretion, levels of cholesterol transport proteins). There can be variability in the levels of exposure each subject has for the administered compounds (e.g., bile acid and a phenylbutyrate compound), differences in the levels of excretion, and in the pharmacokinetics of the compounds in the subjects being treated. Any of the factors described herein may affect drug exposure by the subject. For instance, decreased clearance of the compounds can result in increased drug exposure, while improved renal function can reduce the actual drug exposure. The extent of drug exposure may be correlated with the subject's response to the administered compounds and the outcome of the treatment.

The subject can be e.g., older than about 18 years of age (e.g., between 18-100, 18-90, 18-80, 18-70, 18-60, 18-50, 18-40, 18-30, 18-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 years of age). The subject can have a BMI of between about 18.5-30 kg/m² (e.g., between 18.5-28, 18.5-26, 18.5-24, 18.5-22, 18.5-20, 20-30, 20-28, 20-26, 20-24, 20-22, 22-30, 22-28, 22-26, 22-24, 24-30, 24-28, 24-26, 26-30, 26-28, or 28-30 kg/m²). Having a mutation in any of the ALS-associated genes described herein or presenting with any of the biomarkers described herein may suggest that a subject is at risk for developing ALS. Such subjects can be treated with the methods provided herein for preventative and prophylaxis purposes.

In some embodiments, the subjects have one or more symptoms of benign fasciculation syndrome (BFS) or cramp-fasciculation syndrome (CFS). BFS and CFS are peripheral nerve hyperexcitability disorders, and can cause fasciculation, cramps, pain, fatigue, muscle stiffness, and paresthesia Methods of identifying subjects with these disorders are known in the art, such as by clinical examination and electromyography.

II. Composition

The present disclosure provides methods of treating at least one symptom of ALS in a subject, the methods including administering to the subject a bile acid or a pharmaceutically acceptable salt thereof and a phenylbutyrate compound. In some embodiments, the methods include administering a composition comprising a TURSO and a sodium phenylbutyrate to a subject.

### Bile Acid

As used herein, “bile acid” refers to naturally occurring surfactants having a nucleus derived from cholanic acid substituted with a 3α-hydroxyl group and optionally with other hydroxyl groups as well, typically at the C6, C7 or C12 position of the sterol nucleus. Bile acid derivatives (e.g., aqueous soluble bile acid derivatives) and bile acids conjugated with an amine are also encompassed by the term “bile acid”. Bile acid derivatives include, but are not limited to, derivatives formed at the carbon atoms to which hydroxyl and carboxylic acid groups of the bile acid are attached with other functional groups, including but not limited to halogens and amino groups. Soluble bile acids may include an aqueous preparation of a free acid form of bile acids combined with one of HCl, phosphoric acid, citric acid, acetic acid, ammonia, or arginine. Suitable bile acids include but are not limited to, taurursodiol (TURSO), ursodeoxycholic acid (UDCA), chenodeoxvcholic acid (also referred to as “chenodiol” or “chenic acid”), cholic acid, hyodeoxycholic acid, deoxycholic acid, 7-oxolithocholic acid, lithocholic acid, iododeoxycholic acid, iocholic acid, taurochenodeoxycholic acid, taurodeoxycholic acid, glycoursodeoxycholic acid, taurocholic acid, glycocholic acid, or an analog, derivative, or prodrug thereof.

In some embodiments, the bile acids of the present disclosure are hydrophilic bile acids. Hydrophilic bile acids include but are not limited to, TURSO, UDCA, chenodeoxycholic acid, cholic acid, hyodeoxycholic acid, lithocholic acid, and glycoursodeoxycholic acid. Pharmaceutically acceptable salts or solvates of any of the bile acids disclosed herein are also contemplated. In some embodiments, bases commonly employed to form pharmaceutically acceptable salts of the bile acids of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The terms “tauroursodeoxycholic acid” (TUDCA) and “taurursodiol” (TURSO) are used interchangeably herein.

The bile acid described herein can be TURSO, as shown in formula I (with labeled carbons to assist in understanding where substitutions may be made).

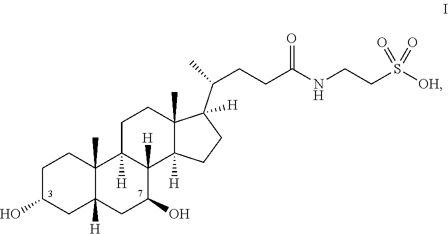

or a pharmaceutically acceptable salt thereof.

The bile acid described herein can be UDCA as shown in formula II (with labeled carbons to assist in understanding where substitutions may be made).

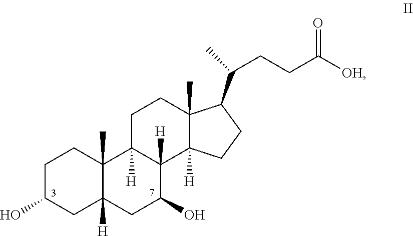

or a pharmaceutically acceptable salt thereof.

Derivatives of bile acids of the present disclosure can be physiologically related bile acid derivatives. For example, any combination of substitutions of hydrogen at position 3 or 7, a shift in the stereochemistry of the hydroxyl group at positions 3 or 7, in the formula of TURSO or UDCA are suitable for use in the present composition.

The “bile acid” can also be a bile acid conjugated with an amino acid. The amino acid in the conjugate can be, but are not limited to, taurine, glycine, glutamine, asparagine, methionine, or carbocysteine. Other amino acids that can be conjugated with a bile acid of the present disclosure include arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid. One example of such a bile acid is a compound of formula III:

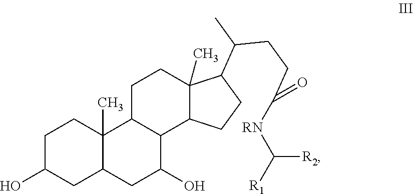

wherein
R is —H or C₁-C₄ alkyl;
R₁ is —CH₂—SO₃R₃, CH₂COOH, or CH₂CH₂COOH, and R₂ is —H,
or R₁ is —COOH and R₂ is —CH₂—CH₂—CONH₂, —CH₂—CONH₂, —CH₂—CH₂—SCH₃, CH₂CH₂CH₂NH(C═NH)NH₂, CH₂(imidazolyl), CH₂CH₂CH₂CH₂NH₂, CH₂COOH, CH₂CH₂COOH, CH₂OH, CH(OH)CH₃, CH₂SH, pyrrolidin-2-yl, CH₃, 2-propyl, 2-butyl, 2-methylbutyl, CH₂(phenyl), CH₂(4-OH-phenyl), or —CH₂—S—CH₂—COOH, and
R₃ is —H or the residue of an amino acid, or a pharmaceutically acceptable analog, derivative, prodrug thereof, or a mixture thereof. One example of the amino acid is a basic amino acid. Other examples of the amino acid include glycine, glutamine, asparagine, methionine, carbocysteine, arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid.

Another example of a bile acid of the present disclosure is a compound of formula IV:

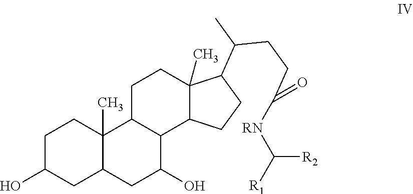

wherein
R is —H or C₁-C₄ alkyl;
R₁ is —CH₂—SO₃R₃, and R₂ is —H;
or R₁ is —COOH and R₂ is —CH₂—CH₂—CONH₂, —CH₂—CONH₂, —CH₂—CH₂—SCH₃, or —CH₂—S—CH₂—COOH; and
R₃ is —H or the residue of a basic amino acid, or a pharmaceutically acceptable analog, derivative, prodrug thereof, or a mixture thereof. Examples of basic amino acids include lysine, histidine, and arginine.

In some embodiments, the bile acid is TURSO. TURSO is an amphiphilic bile acid and is the taurine conjugate form of UDCA. TURSO recovers mitochondrial bioenergetic deficits through incorporating into the mitochondrial membrane, reducing Bax translocation to the mitochondrial membrane, reducing mitochondrial permeability, and increasing the apoptotic threshold of the cell (Rodrigues et al. Biochemistry 42, 10: 3070-3080, 2003). It is used for the treatment of cholesterol gallstones, where long periods of treatment is generally required (e.g., 1 to 2 years) to obtain complete dissolution. It has been used for the treatment of cholestatic liver diseases including primary cirrhosis, pediatric familial intrahepatic cholestasis and primary sclerosing cholangitis and cholestasis due to cystic fibrosis. TURSO is contraindicated in subjects with biliary tract infections, frequent biliary colic, or in subjects who have trouble absorbing bile acids (e.g. ileal disease or resection). Drug interactions may include with substances that inhibit the absorption of bile acids, such as cholestyramine, and with drugs that increase the elimination of cholesterol in the bile (TURSO reduces biliary cholesterol content). Based on similar physicochemical characteristics, similarities in drug toxicity and interactions exist between TURSO and UDCA. The most common adverse reactions reported with the use of TURSO (≥1%) are: abdominal discomfort, abdominal pain, diarrhea, nausea, pruritus, and rash. There are some cases of pruritus and a limited number of cases of elevated liver enzymes.

In some embodiments, the bile acid is UDCA. UDCA, or ursodiol, has been used for treating gallstones, and is produced and secreted endogenously by the liver as a taurine (TURSO) or glycine (GUDCA) conjugate. Taurine conjugation increases the solubility of UDCA by making it more hydrophilic. TURSO is taken up in the distal ileum under active transport and therefore likely has a slightly a longer dwell time within the intestine than UDCA which is taken up more proximally in the ileum. Ursodiol therapy has not been associated with liver damage. Abnormalities in liver enzymes have not been associated with Actigall® (Ursodiol USP capsules) therapy and, Actigall® has been shown to decrease liver enzyme levels in liver disease. However, subjects given Actigall® should have SGOT (AST) and SGPT (ALT) measured at the initiation of therapy and thereafter as indicated by the particular clinical circumstances. Previous studies have shown that bile acid sequestering agents such as cholestyramine and colestipol may interfere with the action of ursodiol by reducing its absorption. Aluminum-based antacids have been shown to adsorb bile acids in vitro and may be expected to interfere with ursodiol in the same manner as the bile acid sequestering agents. Estrogens, oral contraceptives, and clofibrate (and perhaps other lipid-lowering drugs) increase hepatic cholesterol secretion, and encourage cholesterol gallstone formation and hence may counteract the effectiveness of ursodiol.

### Phenylbutyrate Compounds

Phenylbutyrate compound is defined herein as encompassing phenylbutyrate (a low molecular weight aromatic carboxylic acid) as a free acid (4-phenylbutyrate (4-PBA), 4-phenylbutyric acid, or phenylbutyric acid), and pharmaceutically acceptable salts, co-crystals, polymorphs, hydrates, solvates, conjugates, derivatives or pro-drugs thereof. Phenylbutyrate compounds described herein also encompass analogs of 4-PBA, including but not limited to Glyceryl Tri-(4-phenylbutyrate), phenylacetic acid (which is the active metabolite of PBA), 2-(4-Methoxyphenoxy) acetic acid (2-POAA-OMe), 2-(4-Nitrophenoxy) acetic acid (2-POAA-NO2), and 2-(2-Naphthyloxy) acetic acid (2-NOAA), and their pharmaceutically acceptable salts. Phenylbutyrate compounds also encompass physiologically related 4-PBA species, such as but not limited to any substitutions for Hydrogens with Deuterium in the structure of 4-PBA. Other HDAC2 inhibitors are contemplated herein as substitutes for phenylbutyrate compounds.

Physiologically acceptable salts of phenylbutyrate, include, for example sodium, potassium, magnesium or calcium salts. Other example of salts include ammonium, zinc, or lithium salts, or salts of phenylbutyrate with an organ amine, such as lysine or arginine.

In some embodiments of any of the methods described herein, the phenylbutyrate compound is sodium phenylbutyrate. Sodium phenylbutyrate has the following formula:

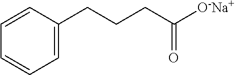

Phenylbutyrate is a pan-HDAC inhibitor and can ameliorate ER stress through upregulation of the master chaperone regulator DJ-1 and through recruitment of other chaperone proteins (See e.g., Zhou et al. J Biol Chem. 286: 14941-14951, 2011 and Suaud et al. JBC. 286:21239-21253, 2011). The large increase in chaperone production reduces activation of canonical ER stress pathways, folds misfolded proteins, and has been shown to increase survival in in vivo models including the G93A SOD1 mouse model of ALS (See e.g., Ryu, H et al. J Neurochem. 93:1087-1098, 2005).

In some embodiments, the combination of a bile acid (e.g., TURSO), or a pharmaceutically acceptable salt thereof, and a phenylbutyrate compound (e.g., sodium phenylbutyrate) has synergistic efficacy when dosed in particular ratios (e.g., any of the ratios described herein), in treating one or more symptoms associated with ALS. The combination can, for example, induce a mathematically synergistic increase in neuronal viability in a strong oxidative insult model (H₂O₂-mediated toxicity) by linear modeling, through the simultaneous inhibition of endoplasmic reticulum stress and mitochondrial stress (See, e.g. U.S. Pat. Nos. 9,872,865 and 10,251,896).

### Formulation

Bile acids and phenylbutyrate compounds described herein can be formulated for use as or in pharmaceutical compositions. For example, the methods described herein can include administering an effective amount of a composition comprising TURSO and sodium phenylbutyrate. The term “effective amount”, as used herein, refer to an amount or a concentration of one or more drugs for a period of time (including acute or chronic administration and periodic or continuous administration) that is effective within the context of its administration for causing an intended effect or physiological outcome. The composition can include about 5% to about 15% w/w (e.g., about 6% to about 14%, about 7% to about 13%, about 8% to about 12%, about 8% to about 11%, about 9% to about 10%, or about 9.7% w/w) of TURSO and about 15% to about 45% w/w (e.g., about 20% to about 40%, about 25% to about 35%, about 28% to about 32%, or about 29% to about 30%, e.g., about 29.2% w/w) of sodium phenylbutyrate. In some embodiments, the composition includes about 9.7% w/w of TURSO and 29.2% w/w of sodium phenylbutyrate.

The sodium phenylbutyrate and TURSO can be present in the composition at a ratio by weight of between about 1:1 to about 4:1 (e.g., about 2:1 or about 3:1). In some embodiments, the ratio between sodium phenylbutyrate and TURSO is about 3:1.

The compositions described herein can include any pharmaceutically acceptable carrier, adjuvant, and/or vehicle. The term “pharmaceutically acceptable carrier or adjuvant” refers to a carrier or adjuvant that may be administered to a patient, together with a compound disclosed herein, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

Compositions of the present disclosure can include about 8% to about 24% w/w of dextrates (e.g., about 9% to about 23%, about 10% to about 22%, about 10% to about 20%, about 11% to about 21%, about 12% to about 20%, about 13% to about 19%, about 14% to about 18%, about 14% to about 17%, about 15% to about 16%, or about 15.6% w/w of dextrates). Both anhydrous and hydrated dextrates are contemplated herein. The dextrates of the present disclosure can include a mixture of saccharides developed from controlled enzymatic hydrolysis of starch. Some embodiments of any of the compositions described herein include hydrated dextrates (e.g., NF grade, obtained from JRS Pharma, Colonial Scientific, or Quadra).

Compositions of the present disclosure can include about 1% to about 6% w/w of sugar alcohol (e.g., about 2% to about 5%, about 3% to about 4%, or about 3.9% w/w of sugar alcohol). Sugar alcohols can be derived from sugars and contain one hydroxyl group (—OH) attached to each carbon atom. Both disaccharides and monosaccharides can form sugar alcohols. Sugar alcohols can be natural or produced by hydrogenation of sugars. Exemplary sugar alcohols include but are not limited to, sorbitol, xylitol, and mannitol. In some embodiments, the composition comprises about 1% to about 6% w/w (e.g., about 2% to about 5%, about 3% to about 4%, or about 3.9% w/w) of sorbitol.

Compositions of the present disclosure can include about 22% to about 35% w/w of maltodextrin (e.g., about 22% to about 33%, about 24% to about 31%, about 25% to about 32%, about 26% to about 30%, or about 28% to about 29% w/w, e.g., about 28.3% w/w of maltodextrin). Maltodextrin can form a flexible helix enabling the entrapment of the active ingredients (e.g., any of the phenylbutyrate compounds and bile acids described herein) when solubilized into solution, thereby masking the taste of the active ingredients. Maltodextrin produced from any suitable sources are contemplated herein, including but not limited to, pea, rice, tapioca, corn, and potato. In some embodiments, the maltodextrin is pea maltodextrin. In some embodiments, the composition includes about 28.3% w/w of pea maltodextrin. For example, pea maltodextrin obtained from Roquette (KLEPTOSE® LINECAPS) can be used.

The compositions described herein can further include sugar substitutes (e.g. sucralose). For example, the compositions can include about 0.5% to about 5% w/w of sucralose (e.g., about 1% to about 4%, about 1% to about 3%, or about 1% to about 2%, e.g., about 1.9% w/w of sucralose). Other sugar substitutes contemplated herein include but are not limited to aspartame, neotame, acesulfame potassium, saccharin, and advantame.

In some embodiments, the compositions include one or more flavorants. The compositions can include about 2% to about 15% w/w of flavorants (e.g., about 3% to about 13%, about 3% to about 12%, about 4% to about 9%, about 5% to about 10%, or about 5% to about 8%, e.g., about 7.3% w/w). Flavorants can include substances that give another substance flavor, or alter the characteristics of a composition by affecting its taste. Flavorants can be used to mask unpleasant tastes without affecting physical and chemical stability, and can be selected based on the taste of the drug to be incorporated. Suitable flavorants include but are not limited to natural flavoring substances, artificial flavoring substances, and imitation flavors. Blends of flavorants can also be used. For example, the compositions described herein can include two or more (e.g., two, three, four, five or more) flavorants. Flavorants can be soluble and stable in water. Selection of suitable flavorants can be based on taste testing. For example, multiple different flavorants can be added to a composition separately, which are subjected to taste testing. Exemplary flavorants include any fruit flavor powder (e.g., peach, strawberry, mango, orange, apple, grape, raspberry, cherry or mixed berry flavor powder). The compositions described herein can include about 0.5% to about 1.5% w/w (e.g., about 1% w/w) of a mixed berry flavor powder and/or about 5% to about 7% w/w (e.g., about 6.3% w/w) of a masking flavor. Suitable masking flavors can be obtained from e.g., Firmenich.

The compositions described herein can further include silicon dioxide (or silica). Addition of silica to the composition can prevent or reduce agglomeration of the components of the composition. Silica can serve as an anti-caking agent, adsorbent, disintegrant, or glidant. In some embodiments, the compositions described herein include about 0.1% to about 2% w/w of porous silica (e.g., about 0.3% to about 1.5%, about 0.5% to about 1.2%, or about 0.8% to about 1%, e.g., 0.9% w/w). Porous silica may have a higher H₂O absorption capacity and/or a higher porosity as compared to fumed silica, at a relative humidity of about 20% or higher (e.g., about 25%, 30%, 35%, 40%, 45%, 50%, 55%, 60%, 65%, 70%, 75%, 80%, 85%, 90%, or 95% or higher). The porous silica can have an H₂O absorption capacity of about 5% to about 40% (e.g. about 20% to about 40%, or about 30% to about 40%) by weight at a relative humidity of about 50%. The porous silica can have a higher porosity at a relative humidity of about 20% or higher (e.g., about 30%, 40%, 50%, 60%, 70%, 80%, 90% or higher) as compared to that of fumed silica. In some embodiments, the porous silica have an average particle size of about 2 μm to about 10 μm (e.g. about 3 μm to about 9 μm, about 4 μm to about 8 μm, about 5 μm to about 8 μm, or about 7.5 μm). In some embodiments, the porous silica have an average pore volume of about 0.1 cc/gm to about 2.0 cc/gm (e.g., about 0.1 cc/gm to about 1.5 cc/gm, about 0.1 cc/gm to about 1 cc/gm, about 0.2 cc/gm to about 0.8 cc/gm, about 0.3 cc/gm to about 0.6 cc/gm, or about 0.4 cc/gm). In some embodiments, the porous silica have a bulk density of about 50 g/L to about 700 g/L (e.g. about 100 g/L to about 600 g/L, about 200 g/L to about 600 g/L, about 400 g/L to about 600 g/L, about 500 g/L to about 600 g/L, about 540 g/L to about 580 g/L, or about 560 g/L). In some embodiments, the compositions described herein include about 0.05% to about 2% w/w (e.g., any subranges of this range described herein) of Syloid®) 63FP (WR Grace).

The compositions described herein can further include one or more buffering agents. For example, the compositions can include about 0.5% to about 5% w/w of buffering agents (e.g., about 1% to about 4% w/w, about 1.5% to about 3.5% w/w, or about 2% to about 3% w/w, e.g. about 2.7% w/w of buffering agents). Buffering agents can include weak acid or base that maintain the acidity or pH of a composition near a chosen value after addition of another acid or base. Suitable buffering agents are known in the art. In some embodiments, the buffering agent in the composition provided herein is a phosphate, such as a sodium phosphate (e.g., sodium phosphate dibasic anhydrous). For example, the composition can include about 2.7% w/w of sodium phosphate dibasic.

The compositions can also include one or more lubricants. For example, the compositions can include about 0.05% to about 1% w/w of lubricants (e.g., about 0.1% to about 0.9%, about 0.2% to about 0.8%, about 0.3% to about 0.7%, or about 0.4% to about 0.6%, e.g. about 0.5% w/w of lubricants). Exemplary lubricants include, but are not limited to sodium stearyl fumarate, magnesium stearate, stearic acid, metallic stearates, talc, waxes and glycerides with high melting temperatures, colloidal silica, polyethylene glycols, alkyl sulphates, glyceryl behenate, and hydrogenated oil. Additional lubricants are known in the art. In some embodiments, the composition includes about 0.05% to about 1% w/w (e.g., any of the subranges of this range described herein) of sodium stearyl fumarate. For example, the composition can include about 0.5% w/w of sodium stearyl fumarate.

In some embodiments, the composition include about 29.2% w/w of sodium phenylbutyrate, about 9.7% w/w of TURSO, about 15.6% w/w of dextrates, about 3.9% w/w of sorbitol, about 1.9% w/w of sucralose, about 28.3% w/w of maltodextrin, about 7.3% w/w of flavorants, about 0.9% w/w of silicon dioxide, about 2.7% w/w of sodium phosphate (e.g. sodium phosphate dibasic), and about 0.5% w/w of sodium stearyl fumerate.

The composition can include about 3000 mg of sodium phenylbutyrate, about 1000 mg of TURSO, about 1600 mg of dextrates, about 400 mg of sorbitol, about 200 mg of sucralose, about 97.2 mg of silicon dioxide, about 2916 mg of maltodextrin, about 746 mg of flavorants (e.g. about 102 mg of mixed berry flavor and about 644 mg of masking flavor), about 280 mg of sodium phosphate (e.g. sodium phosphate dibasic), and about 48.6 mg of sodium stearyl fumerate.

Additional suitable sweeteners or taste masking agents can also be included in the compositions, such as but not limited to, xylose, ribose, glucose, mannose, galactose, fructose, dextrose, sucrose, maltose, steviol glycosides, partially hydrolyzed starch, and corn syrup solid. Water soluble artificial sweeteners are contemplated herein, such as the soluble saccharin salts (e.g., sodium or calcium saccharin salts), cyclamate salts, acesulfam potassium (acesulfame K), and the free acid form of saccharin and aspartame based sweeteners such as L-aspartyl-phenylalanine methyl ester, Alitame® or Neotame®. The amount of sweetener or taste masking agents can vary with the desired amount of sweeteners or taste masking agents selected for a particular final composition.

Pharmaceutically acceptable binders in addition to those described above are also contemplated. Examples include cellulose derivatives including microcrystalline cellulose, low-substituted hydroxypropyl cellulose (e.g. LH 22, LH 21, LH 20, LH 32, LH 31, LH30); starches, including potato starch; croscarmellose sodium (i.e. cross-linked carboxymethylcellulose sodium salt; e.g. Ac-Di-Sol®)) alginic acid or alginates; insoluble polyvinylpyrrolidone (e.g. Polyvidon® CL, Polyvidon® CL-M, Kollidon® CL, Polyplasdone®, XL, Polyplasdone® XL-10); and sodium carboxymethyl starch (e.g. Primogel® and Explotab®).

Additional fillers, diluents or binders may be incorporated such as polyols, sucrose, sorbitol, mannitol, Erythritol®, Tagatose®, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcema® P100, Emcocel®, Vivacel®, Ming Tai® and Solka-Floc®), hydroxypropylcellulose, L-hydroxypropylcellulose (low-substituted) (e.g. L-HPC-CH31, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium sulfate, and calcium carbonate.

The compositions described herein can be formulated or adapted for administration to a subject via any route (e.g. any route approved by the Food and Drug Administration (FDA)). Exemplary methods are described in the FDA's CDER Data Standards Manual, version number 004 (which is available at fda.give/cder/dsm/DRG/drg00301.html).

Pharmaceutical compositions are typically formulated to be compatible with its intended route of administration. Examples of routes of administration include parenteral (subcutaneous, intracutaneous, intravenous, intradermal, intramuscular, intra-articular, intraarterial, intrasynovial, intrasternal, intrathecal, intralesional and intracranial injection or infusion techniques), oral (e.g., inhalation or through a feeding tube), transdermal (topical), transmucosal, and rectal administration.

Pharmaceutical compositions can be in the form of a solution or powder for inhalation and/or nasal administration. In some embodiments, the pharmaceutical composition is formulated as a powder filled sachet. Suitable powders may include those that are substantially soluble in water. Pharmaceutical compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions can be orally administered in any orally acceptable dosage form including, but not limited to, powders, capsules, tablets, emulsions and aqueous suspensions, dispersions and solutions. In the case of powders for oral administration, the powders can be substantially dissolved in water prior to administration. In the case of tablets for oral use, carriers which are commonly used include lactose and corn starch. Lubricating agents, such as magnesium stearate, may be added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, the compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the bile acid and phenylbutyrate compounds. The kit may also include instructions for the physician and/or patient, syringes, needles, box, bottles, vials, etc.

III. Methods of Treatment/Pharmacokinetics

In one aspect, provided herein are methods for treating at least one symptom of ALS in a subject, wherein the method comprises: (a) administering to the subject one or more doses of a composition comprising about 1 gram of Taurursodiol (TURSO) and about 3 grams of sodium phenylbutyrate, (b) determining that the subject has a certain levels of Cₘₐₓ AUC₀₋ₗₐₛₜ, and/or AUC_{0-∞} for sodium phenylbutyrate, and/or certain levels of Cₘₐₓ, AUC₀₋ₗₐₛₜ, and/or AUC_{0-∞} for the metabolite phenylacetate, and (c) administering to the subject an additional dose of the composition.

The methods can include determining that the subject has a Cₘₐₓ for sodium phenylbutyrate of about 3 to about 425 μg/mL (e.g. about 10 to about 425, about 20 to about 425, about 30 to about 425, about 40 to about 425, about 50 to about 425, about 60 to about 425, about 70 to about 425, about 80 to about 425, about 100 to about 425, about 150 to about 425, about 200 to about 425, about 300 to about 425, about 90 to about 170, or about 110 to about 150 μg/mL).

Sodium phenylbutyrate can be rapidly cleared by metabolism (β-oxidation in the liver and kidney) to the primary metabolite phenylacetate (PAA). Accordingly, the methods can include determining that the subject has a Cₘₐₓ for phenylacetate of about 5 to about 50 μg/mL (e.g., about 9 to about 45, about 9 to about 40, or about 15 to about 35 μg/mL). In some embodiments of any of the methods described herein, the Cₘₐₓ is steady-state Cₘₐₓ (e.g, steady-state mean Cₘₐₓ).

The methods can include determining that the subject has an AUC₀₋ₗₐₛₜ for sodium phenylbutyrate of about 20 to about 550 μg*h/mL (e.g. about 40 to about 500, about 60 to about 450, about 80 to about 400, about 100 to about 350 or about 140 to about 300 μg*h/mL). The methods can also include determining that the subject has an AUC₀₋ₗₐₛₜ for phenylacetate of about 20 to about 160 μg*h/mL (e.g. about 30 to about 150, about 40 to about 120, or about 40 to about 80 μg*h/mL). In some embodiments of any of the methods described herein, the AUC₀₋ₗₐₛₜ is steady-state AUC₀₋ₗₐₛₜ (e.g, steady-state mean AUC₀₋ₗₐₛₜ).

In some instances, the methods include determining that the subject has (1) an AUC_{0-∞} for sodium phenylbutyrate of about 25 to about 545 μg*h/mL, and/or (2) an AUC_{0-∞} for phenylacetate of about 21 to about 155 μg*h/mL. For example, the methods can include determining that the subject has an AUC_{0-∞} for sodium phenylbutyrate of about 40 to about 500, about 60 to about 450, about 80 to about 400, about 100 to about 350 or about 140 to about 300 μg*h/mL. The methods can also include determining that the subject has an AUC_{0-∞} for phenylacetate of about 30 to about 150, about 40 to about 120, or about 40 to about 80 μg*h/mL. In some embodiments of any of the methods described herein, the AUC_{0-∞} is steady-state AUC_{0-∞} (e.g., steady-state mean AUC_{0-∞}).

In some embodiments of any of the methods described herein, step (a) can include administering the composition once a day or twice a day for about 1 day to about 40 weeks (e.g. about 3 days to about 38 weeks, about 1 week to about 34 weeks, about 3 weeks to about 32 weeks, about 4 weeks to about 32 weeks, about 6 weeks to about 32 weeks, about 8 weeks to about 32 weeks, about 10 weeks to about 32 weeks, about 10 weeks to about 26 weeks, or about 12 weeks to about 24 weeks). For example, the composition can be administered twice a day for about 9 weeks to about 21 weeks. In some instances, the composition is administered once a day for about 3 weeks followed by twice a day for about 9 weeks to about 21 weeks.

Step (b) of the methods described herein can include obtaining a blood sample from the subject about 30 minutes to about 8 hours (e.g. about 1, 2, 3, 4, 5, 6, or 7 hours) after the last dose of the composition.

In another aspect, provided herein are methods of treating at least one symptom of ALS in a subject, the methods include (a) administering to the subject one or more doses of a composition comprising about 1 gram of TURSO and about 3 grams of sodium phenylbutyrate; (b) determining a plasma concentration of one or more bile acids in the subject; and (c) administering to the subject an additional dose of the composition. For example, the methods can include determining the plasma concentration of TURSO, UDCA, GUDCA, Cholic acid (CA), CDCA (Chenodeoxycholic acid), DCA (Deoxycholic acid), Glycocholic acid (GCA), Glycodeoxycholic acid (GDCA), Taurocholic acid (TCA), Taurochenodeoxycholic acid (TCDCA), or Taurodeoxycholic acid (TDCA). In some embodiments, the plasma concentration is steady-state plasma concentration. In some embodiments, step (b) can include determining the plasma concentration about 30 minutes to about 8 hours (e.g. about 1, 2, 3, 4, 5, 6, or 7 hours) after the last dose of the composition.

In some embodiments of the above aspect, the methods include determining the plasma concentration of TURSO in the subject about one hour after the last dose of the composition, where the plasma concentration of TURSO is about 20 to about 2570 ng/mL (e.g., about 20 to about 2000, about 20 to about 1800, about 20 to about 1500, about 20 to about 1100, about 20 to about 1045, about 40 to about 800, about 60 to about 700, or about 88 to about 540 ng/mL). The plasma concentration of TURSO can also be determined about 4 hours after the last dose of the composition where the plasma concentration is about 20 to about 3250 ng/mL (e.g. about 20 to about 2800, about 20 to about 2500, about 20 to about 2000, about 20 to about 1800, about 20 to about 1500, about 20 to about 1200, about 20 to about 1125, about 50 to about 1000, about 80 to about 900, about 100 to about 800, or about 155 to about 785 ng/mL).

In some embodiments of the above aspect, the methods include determining the plasma concentration of UDCA in the subject about one hour after the last dose of the composition, where the plasma concentration of UDCA is about 20 to about 6020 ng/mL (e.g. about 20 to about 5500, about 20 to about 5000, about 20 to about 4500, about 20 to about 4000, about 20 to about 3500, about 20 to about 3000, about 20 to about 2500, about 20 to about 2000, about 20 to about 1955, about 50 to about 1800, about 80 to about 1500, or about 285 to about 1125 ng/mL). The plasma concentration of UDCA can also be determined about 4 hours after the last dose of the composition where the plasma concentration is about 20 to about 7340 ng/mL (e.g., about 20 to about 7000, about 20 to about 6000, about 20 to about 5000, about 20 to about 4000, about 20 to about 3000, about 20 to about 2550, about 50 to about 2000, about 100 to about 1500 or about 305 to about 1395 ng/mL).

In some embodiments of the above aspect, the methods include determining the plasma concentration of GUDCA in the subject about one hour after the last dose of the composition, where the plasma concentration of GUDCA is about 20 to about 4600 ng/mL (e.g. about 20 to about 4000, about 20 to about 3500, about 20 to about 3000, about 40 to about 2500, about 65 to about 2085, or about 340 to about 1635 ng/mL). The plasma concentration of GUDCA can also be determined about 4 hours after the last dose of the composition where the plasma concentration is about 20 to about 5290 ng/mL (e.g. about 40 to about 4500, about 80 to about 4000, about 150 to about 3500, about 320 to about 2315 ng/mL, or about 530 to about 1915 ng/mL).

In some embodiments of the above aspect, the methods further include prior to step (a), determining a baseline plasma concentration of the bile acid in the subject. For example, the methods can include determining a baseline plasma concentration of TURSO in the subject, where the baseline plasma concentration of TURSO is about 20 to about 577 ng/mL (e.g. about 20 to about 400, about 20 to about 300, about 20 to about 200, or about 20 to about 125 ng/mL). The methods can include determining a baseline plasma concentration of UDCA in the subject, where the baseline plasma concentration of UDCA is about 20 to about 5970 ng/mL (e.g., about 20 to about 5000, about 20 to about 4000, about 20 to about 3000, about 20 to about 2000, about 20 to about 1000, about 20 to about 825, about 20 to about 500, or about 20 to about 52 ng/mL). The methods can include determining a baseline plasma concentration of GUDCA in the subject, where the baseline plasma concentration of GUDCA is about 20 to about 4540 ng/mL (e.g., about 20 to about 4000, about 20 to about 3000, about 20 to about 2000, about 20 to about 755, or about 25 to about 180 ng/mL).

In some embodiments of any of the methods described herein, the subject has fasted for a period of time prior to receiving a dose of the compositions. For example, step (a) of the methods can include administering a dose of the composition more than two hours after the subject has consumed food, or more than one hour before the subject consumes food. In some embodiments, the subject has consumed food within two hours (e.g., within one hour, or within 30 minutes) of receiving the administration of the composition.

In a further aspect, provided herein are methods of increasing the plasma concentration of a bile acid (e.g. any of the bile acids described herein) in a subject, the method comprising administering to the subject one or more doses of a composition comprising about 3 grams of sodium phenylbutyrate and about 1 gram of TURSO. The composition can be administered according to any suitable dosing regimen disclosed herein. In some embodiments, the bile acid is selected from TURSO. UDCA and GUDCA. For example, the bile acid can be TURSO, and the plasma concentration of TURSO after administration of the composition is about 20 to about 3250 ng/mL (e.g. any of the subranges within this range described herein). The bile acid can be UDCA, and the plasma concentration of UDCA after administration of the composition is about 20 to about 7340 ng/mL (e.g. any of the subranges within this range described herein). The bile acid can also be GUDCA, and the plasma concentration of GUDCA after administration of the composition is about 20 to about 5290 ng/mL (e.g. any of the subranges within this range described herein).

Also provided herein are methods of administering to a subject (e.g. a healthy subject) who has been fasting for 24 hours a composition comprising about 1 gram of Taurursodiol (TURSO) and about 3 grams of sodium phenylbutyrate and measuring one or more pharmacokinetic parameters, such as, Tₘₐₓ, Cₘₐₓ, and AUC₀₋ₗₐₛₜ. In some embodiments, the subject has a Tₘₐₓ for sodium phenylbutyrate of about 0.25 hours to about 0.50 hours. In some embodiments, the subject has a Cₘₐₓ for sodium phenylbutyrate of about 64.4 μg/mL to about 260 μg/mL. In some embodiments, the subject has an AUC₍₀₋ₗₐₛₜ₎ for sodium phenylbutyrate of about 73.5 μg*h/mL to about 423 μg*h/mL. In some embodiments, the subject has an AUC_{(0-∞)} for sodium phenylbutyrate of about 74.8 μg*h/mL to about 425 μg*h/mL. In some embodiments, the subject has a Tₘₐₓ for TURSO of about 1.50 hours to about 10.00 hours. In some embodiments, the subject has a Cₘₐₓ for TURSO of about 0.219 μg/mL to about 1.74 μg/mL. In some embodiments, the subject has an AUC₍₀₋ₗₐₛₜ₎ for TURSO of about 1.18 μg*h/mL to about 11.6 μg*h/mL. In some embodiments, the subject has an AUC_{(0-∞)} for TURSO of about 1.80 μg*h/mL to about 6.67 μg*h/mL. In any of the embodiments described above, the Tₘₐₓ can be a steady-state Tₘₐₓ (e.g., steady-state mean Tₘₐₓ). In any of the embodiments described above, the Cₘₐₓ can be a steady-state Cₘₐₓ (e.g., steady-state mean Cₘₐₓ). In any of the embodiments described above, the AUC₍₀₋ₗₐₛₜ₎ can be a steady-state AUC₍₀₋ₗₐₛₜ₎ (e.g., steady-state mean AUC₍₀₋ₗₐₛₜ₎). In any of the embodiments described above, the AUC_{(0-∞)} can be a steady-state AUC_{(0-∞)} (e.g., steady-state mean AUC_{(0-∞)}).

Also provided herein are methods of administering to a subject (e.g. a healthy subject) who has been fasting for 24 hours a composition comprising about 1 gram of Taurursodiol (TURSO) and about 3 grams of sodium phenylbutyrate and measuring one or more pharmacokinetic parameters, such as, Tₘₐₓ, Cₘₐₓ, and AUC₀₋ₗₐₛₜ for one or more metabolites of sodium phenylbutyrate and/or TURSO. Specifically, phenylacetate is considered a metabolite of sodium phenylbutyrate. Further, under physiological conditions, enterohepatic recirculation results in active de-conjugation of TURSO to UDCA by intestinal microflora, and reconjugation of UDCA in the liver with glycine or taurine (GUDCA and TURSO, respectively). In some embodiments, the subject has a Tₘₐₓ for phenylacetate of about 1.50 hours to about 3.50 hours. In some embodiments, the subject has a Cₘₐₓ for phenylacetate of about 13.3 μg/mL to about 42.3 μg/mL. In some embodiments, the subject has an AUC₍₀₋ₗₐₛₜ₎ for phenylacetate of about 43.8 μg*h/mL to about 141 μg*h/mL. In some embodiments, the subject has an AUC_{(0-∞)} for phenylacetate of about 45.2 μg*h/mL to about 142 μg*h/mL. In some embodiments, the subject has a Tₘₐₓ for UDCA of about 0.25 hours to about 20.00 hours. In some embodiments, the subject has a Cₘₐₓ for UDCA of about 195 ng/mL to about 1380 ng/mL. In some embodiments, the subject has an AUC₍₀₋ₗₐₛₜ₎ for UDCA of about 1970 ng*h/mL to about 14900 ng*h/mL. In some embodiments, the subject has an AUC_{(0-∞)} for UDCA of about 11300 ng*h/mL. In some embodiments, the subject has a Tₘₐₓ for GUDCA of about 6.00 hours to about 20.00 hours. In some embodiments, the subject has a Cₘₐₓ for GUDCA of about 143 ng/mL to about 1420 ng/mL. In some embodiments, the subject has an AUC₍₀₋ₗₐₛₜ₎ for GUDCA of about 1840 ng*h/mL to about 12000 ng*h/mL. In any of the embodiments described above, the Tₘₐₓ can be a steady-state Tₘₐₓ (e.g., steady-state mean Tₘₐₓ). In any of the embodiments described above, the Cₘₐₓ can be a steady-state Cₘₐₓ (e.g., steady-state mean Cₘₐₓ). In any of the embodiments described above, the AUC₍₀₋ₗₐₛₜ₎ can be a steady-state AUC₍₀₋ₗₐₛₜ₎ (e.g., steady-state mean AUC₍₀₋ₗₐₛₜ₎). In any of the embodiments described above, the AUC_{(0-∞)} can be a steady-state AUC_{(0-∞)} (e.g., steady-state mean AUC_{(0-∞)}).

Also provided herein methods of administering to a subject (e.g. a healthy subject) who has been fed a high-fat diet a composition comprising about 1 gram of Taurursodiol (TURSO) and about 3 grams of sodium phenylbutyrate and measuring one or more pharmacokinetic parameters, such as, Tₘₐₓ, Cₘₐₓ, and AUC₀₋ₗₐₛₜ. In some embodiments, the subject has a Tₘₐₓ for sodium phenylbutyrate of about 0.25 hours to about 1.50 hours. In some embodiments, the subject has a Cₘₐₓ for sodium phenylbutyrate of about 12.9 μg/mL to about 70.5 μg/mL. In some embodiments, the subject has an AUC₍₀₋ₗₐₛₜ₎ for sodium phenylbutyrate of about 34.6 μg*h/mL to about 198 μg*h/mL. In some embodiments, the subject has an AUC_{(0-∞)} for sodium phenylbutyrate of about 76.2 μg*h/mL to about 200 μg*h/mL. In some embodiments, the subject has a Tₘₐₓ for TURSO of about 4.50 hours to about 10.00 hours. In some embodiments, the subject has a Cₘₐₓ for TURSO of about 0.435 μg/mL to about 2.16 μg/mL. In some embodiments, the subject has an AUC₍₀₋ₗₐₛₜ₎ for TURSO of about 2.45 μg*h/mL to about 22.4 μg*h/mL. In some embodiments, the subject has an AUC_{(0-∞)} for TURSO of about 3.09 μg*h/mL to about 5.83 μg*h/mL. In any of the embodiments described above, the Tₘₐₓ can be a steady-state Tₘₐₓ (e.g., steady-state mean Tₘₐₓ). In any of the embodiments described above, the Cₘₐₓ can be a steady-state Cₘₐₓ (e.g., steady-state mean Cₘₐₓ). In any of the embodiments described above, the AUC₍₀₋ₗₐₛₜ₎ can be a steady-state AUC₍₀₋ₗₐₛₜ₎ (e.g., steady-state mean AUC₍₀₋ₗₐₛₜ₎). In any of the embodiments described above, the AUC_{(0-∞)} can be a steady-state AUC_{(0-∞)} (e.g., steady-state mean AUC_{(0-∞)}).

Also provided herein methods of administering to a subject (e.g. a healthy subject) who has been fed a high-fat diet a composition comprising about 1 gram of Taurursodiol (TURSO) and about 3 grams of sodium phenylbutyrate and measuring one or more pharmacokinetic parameters, such as, Tₘₐₓ, Cₘₐₓ, and AUC₀₋ₗₐₛₜ for one or more metabolites of sodium phenylbutyrate and/or TURSO. In some embodiments, the subject has a Tₘₐₓ for phenylacetate of about 2.00 hours to about 4.50 hours. In some embodiments, the subject has a Cₘₐₓ for phenylacetate of about 8.39 μg/ml to about 36.5 μg/mL. In some embodiments, the subject has a AUC₍₀₋ₗₐₛₜ₎ for phenylacetate of about 30.6 μg*h/mL to about 128 μg*h/mL. In some embodiments, the subject has an AUC_{(0-∞)} for phenylacetate of about 31.3 μg*h/mL to about 130 μg*h/mL. In some embodiments, the subject has a Tₘₐₓ for UDCA of about 6.00 hours to about 24.00 hours. In some embodiments, the subject has a Cₘₐₓ for UDCA of about 181 ng/mL to about 6250 ng/mL. In some embodiments, the subject has an AUC₍₀₋ₗₐₛₜ₎ for UDCA of about 1590 ng*h/mL to about 33400 ng*h/mL. In some embodiments, the subject has an AUC_{(0-∞)} for UDCA of about 8580 ng*h/mL. In some embodiments, the subject has a Tₘₐₓ for GUDCA of about 0.50 hours to about 24.00 hours. In some embodiments, the subject has a Cₘₐₓ for GUDCA of about 114 ng/mL to about 2430 ng/mL. In some embodiments, the subject has an AUC₍₀₋ₗₐₛₜ₎ for GUDCA of about 900 ng*h/mL to about 18400 ng*h/mL. In any of the embodiments described above, the Tax can be a steady-state Tₘₐₓ (e.g., steady-state mean Tₘₐₓ). In any of the embodiments described above, the Cₘₐₓ can be a steady-state Cₘₐₓ (e.g., steady-state mean Cₘₐₓ). In any of the embodiments described above, the AUC₍₀₋ₗₐₛₜ₎ can be a steady-state AUC₍₀₋ₗₐₛₜ₎ (e.g., steady-state mean AUC₍₀₋ₗₐₛₜ₎). In any of the embodiments described above, the AUC_{(0-∞)} can be a steady-state AUC_{(0-∞)} (e.g., steady-state mean AUC_{(0-∞)}).

### EXAMPLES

The invention will be described in greater detail by way of specific examples. The following examples are offered for illustrative purposes, and are not intended to limit the invention in any manner. Those of skill in the art will readily recognize a variety of non-critical parameters, which can be changed or modified to yield essentially the same results. It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable sub-combination.

### Example 1: Pharmacokinetics Studies

The pharmacokinetics of AMX0035 are based on plasma concentration data from two studies: a single dose PK study in healthy volunteers, and sparse sampling from patients in a clinical efficacy and safety study.

### Protocols

1. Protocol AMX3500: Evaluation of the safety, tolerability, efficacy and activity of AMX0035, a fixed combination of phenylbutyrate (PB) and tauroursodeoxycholic acid (TUDCA), for treatment of Amyotrophic Lateral Sclerosis (ALS)
   28-week multi-center, randomized, double-blind, placebo-controlled phase II trial examining the safety, tolerability, efficacy, pharmacokinetics and biological activity of AMX0035
   Population: 132 males or females between 18-80 years of age with sporadic or familial ALS, vital capacity >60% of predicted, and onset of ALS symptoms within the last 18 months.
   Treatment: Subjects randomly assigned in a 2:1 ratio to receive oral (or feeding tube) AMX0035 (approximately 88 subjects) or placebo (approximately 44 subjects). Treatment is administered as one sachet daily for the initial 3 weeks and then increased if tolerated to one sachet twice daily. Sachet in the active treatment group contains 1 g TUDCA and 3 g PB.
   Pharmacokinetic sampling: Single blood samples for analysis of PB, phenylacetate (PAA), TUDCA, UDCA and GUDCA plasma concentrations are collected at the baseline visit (pre-dose) and the 12 and 24 week treatment visits. Sampling times are randomly assigned to occur at either 1 or 4 hours post-dose at the 12 week visit and the other time at the 24 week visit.
   Treatment duration: 24 weeks
2. Protocol A35-002: A Phase I study of single dose oral AMX0035 under the fasted and fed state in healthy volunteers to evaluate plasma pharmacokinetics
   Phase I, open label, two period cross-over single dose trial of AMX-0035 in healthy adult volunteers examining the pharmacokinetics of PB, TUDCA and major metabolites following single oral dose administration of AMX-0035 with and without food.
   Population: 14 male or female healthy volunteers between 40 and 65 years of age and 18.5 and 32 kg/m2 body mass index.
   Treatment: Subjects receive a single-dose of one sachet (1 g TUDCA and 3 g PB) of AMX0035 under fasting (overnight and 4 hours post-dose) and fed (standard high-fat breakfast 30 minutes prior to dose) conditions with a minimum 4 day washout between treatments. The order of administration with and without food is randomly determined
   Pharmacokinetic sampling: Blood samples for analysis of PB, PAA, TUDCA, UDCA, and GUDCA plasma concentrations are collected pre-dose and 0.25, 0.5, 0.75, 1, 1.5, 2, 2.5, 3, 3.5, 4, 4.5, 5, 6, 7, 8, 10, 12, 16, 20 and 24 hours following the single-dose of AMX0035 on both study days. Additionally, serial plasma samples are obtained on the day prior to the first dose of AMX0035 to characterize endogenous concentrations of TUDCA and metabolites.

### Pharmacokinetic Data Characteristics

Protocol AMX3500: sparse sampling in 87 subjects with ALS. Approximately 2 samples are collected for each subject, 1 or 4 hours post dose following 12 and 24 weeks of treatment.
Protocol A35-002: rich sampling in 14 healthy adults. Approximately 21 samples are collected over 24 hours for each subject on two occasions, once following a high fat meal and the other under fasting conditions.

### Results

The primary metabolite of sodium phenylbutyrate (PB) is phenylacetate (PAA). PAA occurs endogenously as a phenylalanine metabolite. Population-PK analysis indicates that PAA has non-linear (saturable) elimination.

Population PK analysis has shown an inverse relationship between PAA exposure and body weight. There is a potential additive effect of fasting to low body weight, in terms of increased PAA exposure.

TURSO, UDCA (ursodiol) and GUDCA (glycoursodeoxycholic acid) are naturally occurring hydrophilic bile acids and represent a minor fraction of the total bile acid pool in humans. All three entities are subject to extensive enterohepatic circulation. Oral administration of TURSO results in increased plasma levels of all three entities.

The pharmacokinetic parameters after a single dose of ALBRIOZA under fasted conditions in healthy subjects are shown for phenylbutyrate and the primary metabolite PAA in Table 1 and for ursodoxicoltaurine and its metabolites in Table 2.

TABLE 1
Geometric Mean (CV%) Pharmacokinetic Parameters for Phenylbutyrate (PB)
and Phenylacetate (PA) After Single Oral Doses of ALBRIOZA (3 g
phenylbutyrate and 1 g ursodoxicoltaurine) in n = 14 Healthy Subjects Under
Fasted Conditions

| | Cmax | Tmax* | T1/2 | AUC_{(0-inf)} | CL/F | |
|---|---|---|---|---|---|---|
| | (μg/mL) | (h) | (h) | (μg.h/ml) | (mL/min) | Vz/F (L) |
| PB | 188 | 0.500 | 0.461 (15.1) | 237 (44.9) | 211 | 8.4 |
| N = 13 | (39.0) | (0.25- | | | (44.9) | (45.8) |
| | | 0.50) | | | | |
| PAA | 26.4 | 2.500 | 0.813 | 83.4 | N/A** | N/A** |
| N = 13 | (30.7) | (1.50- | (11.5)** | (37.4) | | |
| | | 3.50) | | | | |

Cmax = maximum plasma concentration; Tmax = time to maximum concentration; ; T1/2 = half-life; AUC(0-inf) = area under the concentration-time curve from time 0 to infinity; CL/F = oral clearance; Vz/F = apparent volume of distribution.
*Median (range);
**terminal phase T1/2, based on population-PK analysis, the metabolite appears to have non-linear (saturable) elimination
NA not applicable.

TABLE 2
Geometrie Mean (CV%) Pharmacokinetic Parameters for Ursodoxicoltaurine
(TURSO) and Derivatives, UDCA and GUDCA, After Single Oral Doses of
ALBRIOZA (1 g ursodoxicoltaurine and 3 g phenylbutyrate) in n = 14 Healthy
Subjects Under Fasted Conditions

| | Cmax | Tmax** | | AUG₍₀₋ₗₐₛₜ₎ | | |
|---|---|---|---|---|---|---|
| | (ng/ml) | (h) | T1/2 | (ng.h/ml) | CL/F | |
| | N= 13 | N= 13 | (h) | N= 13 | (mL/min) | Vz/F (L) |
| TURSO | 741 (71.6) | 4.5 | 4.34 (49.7) | 4360 (79.4) | 4260 (58.9) | 1600 (22.1) |
| | | (1.5 ~ 10.0) | | | | |
| | | | N = 5*** | | N = 5*** | N = 5*** |
| UDCA | 639 (73.0) | 6.0 | NC | 5540 (72.5) | NA | NA |
| | | (0.25 - 20.0) | | | | |
| GUDCA | 381 (76.6) | 16.0 | NC | 5060 (88.2) | NA | NA |
| | | (6.0 - 20.0) | | | | |

Cmax = maximum plasma concentration; Tmax = time to maximum concentration; TI/2 = half-life; AUC(0-last) = area under the concentration-time curve from time 0 to the last measured concentration (samples were collected for 24 h post-dose);; CL/F = oral clearance; Vz/F″ = apparent volume of distribution.
*PK parameters derived from time-matched, baseline corrected plasma concentrations to account for endogenous levels
**Median (range),
***Reduced number of subjects contributing to the PK parameter value, due to insufficient number of samples collected in the terminal elimination phase
NC = not calculated due to insufficient number of samples collected in the terminal elimination phase
NA = not applicable

Steady-state plasma concentrations of ursodoxicoltaurine UDCA and GUDCA following twice-daily administration of ALBRIOZA in ALS patients are shown in Table 3. Oral administration of TURSO results in increased plasma levels of all three entities.

TABLE 3
Steady-state plasma concentrations of TURSO, UDCA and GUDCA following
twice daily administration of AMX0035 in ALS patients

| | TURSO* | UDCA* | GUDCA* |
|---|---|---|---|
| | ng/ml | ng/ml | ng/ml |
| Predose (Baseline Visit), | 39.7 (82.93) | 137.9 (682.55) | 208.9 (540.96) |
| n = 77 | n = 10/77 | n = 34/77 | n = 66/77 |
| | with measurable | with measurable | with measurable |
| | plasma levels | plasma levels | plasma levels |
| 1 hr Post-dose | 447.5 (595.25) | 909.9 (1046.64) | 1074.3 (1005.89) |
| (Week 12 and Week 24 Pooled), | n = 71/77 | n = 72/77 | n = 77/77 |
| n = 67 | with measurable | with measurable | with measurable |
| | plasma levels | plasma levels | plasma levels |
| 4 hrs Post-dose | 566.1 (558.25) | 1168.9 (1376.14) | 1319.2 (989.37) |
| (Week 12 and Week 24 Pooled), | n = 74/77 | n = 74/77 | n = 76/77 |
| n = 70 | with measurable | with measurable | with measurable |
| | plasma levels | plasma levels | plasma levels |

*Plasma levels recorded as below measurable levels (<20 ng/ml) were set at 20 ng/ml. For all plasma concentration values other than Pre-Dose (Baseline): maximum number of samples with below measurable levels is 10.

### Absorption:

Following oral administration of a single dose of AMX0035 in healthy subjects under fasting conditions (as described in Example 2 above), sodium phenylbutyrate was rapidly absorbed and reached a mean Cₘₐₓ of 188 μg/mL (range of 64.4 to 260) by a median time of 1 hour. The estimated steady-state mean Cₘₐₓ of sodium phenylbutyrate for ALS patients, based on population PK, is 131 μg/mL (range of 3.8 to 423).

Under physiological conditions, bile acids in the intestine are absorbed primarily in the distal portion (ileum) via both passive diffusion (unconjugated bile acids), and active uptake (conjugated bile acids) by the apical sodium-dependent transporter (ASBT). Specific transporters in the intestinal enterocytes ensure bile acids are directed to the liver via the portal vein, where they are subject to extensive enterohepatic recirculation. Bile acids can be recycled 4-12 times per day between hepatocytes in the liver and enterocytes in the intestine. Following oral administration of a single dose of AMX0035 in healthy subjects under fasting conditions, TURSO reached a mean Cm of 871 ng/mL (range of 219 to 1740) by a median time of 4.5 hours. TURSO plasma concentration profiles in many subjects had 2 to 3 peaks, consistent with bile acid storage and release upon a meal/snack.

### Effect of Food

Administration to healthy volunteers of a single dose of 3 g sodium phenylbutyrate and 1 g TURSO with a high-fat, high-calorie meal (approximately 800-1000 calories: 500-600, 250, and 150 calories from fat, carbohydrate, and protein, respectively) decreased the rate and extent of absorption of sodium phenylbutyrate (Cmax and AUC decreased 75% and 55%, respectively). A high-fat, high-calorie meal did not affect the Cmax for TURSO, but the exposure (AUC) increased by 46%. In the Phase II safety and efficacy study, patients were advised to take the drug before a meal.

### Distribution

Plasma protein binding for sodium phenylbutyrate and ursodoxicoltaurine, when co-administered in vitro, is 82% and 98%, respectively.

Under physiological conditions, less than 10% of the total bile acid pool reaches systemic circulation, as the liver clears the majority from the hepatic circulation for reuse. Serum concentration of bile acid reflects a balance between intestinal input and hepatic extraction, with hepatic extraction of conjugated bile acids known to be more efficient than that of unconjugated.

### Metabolism:

Sodium phenylbutyrate is rapidly cleared by metabolism (β-oxidation in the liver and kidney) to the primary metabolite phenylacetate (PAA), which may have pharmacological activity. Plasma concentrations of metabolite PAA in healthy subjects were evident from 0.25 h in all 13 dosed subjects, reaching a mean Cₘₐₓ of 26.44 μg/mL (range of 13.3 to 42.3) by a median time of 2.5 hours. The estimated steady-state mean Cm, for ALS patients, based on population PK analysis, was 24.1 μg/mL (range of 9.08 to 46.3).

Phenylacetate is rapidly conjugated with glutamine via acetylation, in the liver and kidney, to form phenylacetylglutamine, which is excreted by the kidneys.

Under physiological conditions, enterohepatic recirculation results in active de-conjugation of TURSO to UDCA by intestinal microflora, and reconjugation of UDCA in the liver with glycine or taurine (GUDCA and ursodoxicoltaurine, respectively); about 95% of bile acids in the intestine are (re)absorbed into enterohepatic circulation. All metabolites detected in human hepatocytes in vitro that were ascribed to TURSO were also detected in rat and minipig hepatocytes.

### Elimination

Phenylacetate shows non-linear pharmacokinetics characterized by saturable metabolism. By 6 hours post-dose, both phenylbutyrate and phenylacetate were eliminated from systemic circulation, with estimated terminal half-life of 0.46 h and 0.81 h, respectively. Based on this rapid elimination of PB and PA, there was no accumulation in plasma after once- or twice-daily dosing in patients. Other minor metabolites of phenylbutyrate have been identified. The majority of administered sodium phenylbutyrate (˜80-100%) is excreted in the urine within 24 hours as the conjugated product, phenylacetylglutamine.

Terminal half-life of the bile acids could not be reliably determined in most subjects due primarily to insufficient duration of sampling in the terminal elimination phase (see also Tables 6 and 7 above). Comparison across studies (single-dose pharmacokinetic study, and sparse sampling data from the Phase II safety and efficacy study) indicates there appears to be little accumulation of ursodoxicoltaurine after twice-daily dosing, while there was substantial accumulation of UDCA and GUDCA.

Bile acids not (re)absorbed in the intestine (5% under physiological conditions) are further modified bacterially before excretion primarily in the feces. Bile acids found in feces are unconjugated, and consist primarily of the hydrophobic secondary bile acids lithocholic acid and deoxycholic acid, as well as a complex mixture of others. With administration of ursodoxicoltaurine, UDCA levels in feces are known to be increased.

### Special Populations and Conditions

Geriatrics

Model-based analysis showed no significant difference in the pharmacokinetics of sodium phenylbutyrate or its metabolite, phenylacetate, in ALS patients aged 65-76 versus ALS patients less than 65 years of age (number of patients included in dataset: n=18 vs n=39, respectively). There was no difference in steady-state plasma concentration of ursodoxicoltaurine or its major derivatives UDCA and GUDCA in ALS patients aged 65-76 versus ALS patients less than 65 years of age (number of patients included in dataset: n=24-27, vs n=78-82, respectively).

Sex

Following single oral administrations of ALBRIOZA to healthy volunteers, differences in exposure to sodium phenylbutyrate and phenylacetate were observed between males (N=8) and females (N=6), that were consistent across the fed and fasted food conditions, about 35% and 31% higher exposure levels in females when compared to males, respectively. However, due to the moderate variability between the subjects, this difference was not found to be statistically significant. Similarly, sex was not identified as a significant covariate affecting sodium phenylbutyrate or phenylacetate PK parameters in a pharmacometric analysis across studies in healthy subjects and ALS patients (N=26 females and 56 males contributing to dataset).

Following single oral administrations of ALBRIOZA to healthy volunteers, differences in exposure to ursodoxicoltaurine and UDCA were observed between males (N=8) and females (N=6), that were consistent across both food conditions: about 55% higher exposure seen in females for ursodoxicoltaurine, and about 29% lower exposure in females for UDCA, when compared to males. This effect was statistically significant for ursodoxicoltaurine AUC(0-last) only. No such effects were seen for GUDCA. There were no consistent discernible effect of sex on steady-state plasma concentrations of ursodoxicoltaurine, UDCA and GUDCA in ALS patients (N=34 females; 75 males).

Hepatic Insufficiency

Because sodium phenylbutyrate and ursodoxicoltaurine are metabolized in the liver and kidney, increased plasma levels are anticipated with hepatic impairment. Ursodoxicoltaurine and its derivatives such as UDCA and GUDCA are biliary salts and are recycled by the enterohepatic recirculation and stored in the gallbladder. Lithocholic acid, one of products of bile acid metabolism in the intestine, is known to have potent toxic properties. While not observed in the Phase 2 safety and efficacy study, increases in serum levels of lithocholic acid have been reported with TURSO administration, and hepatic insufficiency is anticipated to contribute to potential for harm.

Renal Insufficiency

The Phase II safety and efficacy study included 32 ALS patients with mild renal impairment (estimated glomerular filtration rate (eGFR 60-90 mL/minute). Model-based analysis showed no significant correlation in the pharmacokinetics of sodium phenylbutyrate or its metabolite, phenylacetate, in healthy subjects and ALS patients with normal renal function and mild renal insufficiency (eGFR greater than 60 mL/min). There was no discernible difference in steady-state plasma concentration of ursodoxicoltaurine or its major metabolites UDCA and GUDCA between ALS patients with mild renal insufficiency (eGFR greater than 60 mL/min and less than 90 mL/min=71-76). However, these data are inconclusive because i) a limited number of patients (N=8) had eGFR <75, and ii) eGFR is an overestimate of renal function in ALS patients.

The main terminal metabolite, phenylacetylglutamine, is excreted by the kidney, and additionally phenylbutyrate and its major metabolite phenylacetate are metabolized in the kidney and liver. Therefore, increased plasma levels of phenylbutyrate and metabolites are anticipated with renal impairment. As well, renal insufficiency may alter distribution and efficacy of TURSO, due to high protein binding (˜99%) with ALBRIOZA.

### Example 2: Phase 1 Study of Single Dose Oral AMX0035 Under the Fasted and Fed State in Healthy Volunteers

### Objective

The primary objective of the study was to determine the plasma levels and PK parameters of sodium PB, TURSO and major metabolites following single oral dose administration of AMX0035 in a population of male and female subjects age >40 years.

The secondary objectives of the study were to evaluate the effect of a high-fat standardized breakfast on the extent and rate of absorption of PB. TURSO and active metabolites in healthy subjects; to evaluate effect of gender on the extent and rate of absorption of PB, TURSO and active metabolites in healthy subjects; and to quantify relationship between plasma concentrations of PB and TURSO and active metabolites on QT/corrected QT interval (QTc) prolongation and proarrhythmic potential of single dose AMX0035 in healthy subjects.

### Overall Study Design and Plan

This was a Phase I, open label, 2 sequence, 2-period, cross-over single dose trial of AMX0035 in healthy subjects. A total of 14 healthy subjects were enrolled in order to have no fewer than 12 subjects complete. A predefined mix of gender was selected. Subjects received a single oral dose of AMX0035 on two occasions, with a washout between periods of no fewer than 4 days under fasted conditions or after a high-fat standardized meal in a balanced two-period cross-over design.

Treatment Period 1 was preceded with a 24 h matched sampling for some time points to confirm endogenous levels of TURSO in each subject.

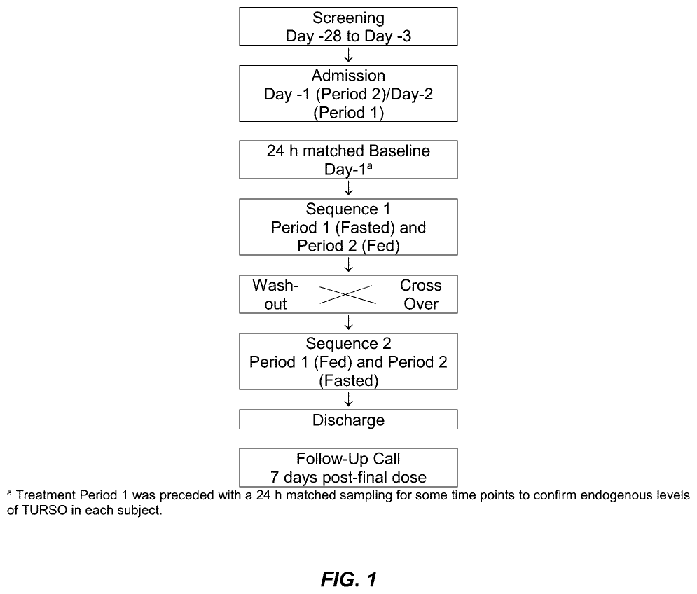

Each study period followed the same study design (FIG. 1). Subjects were screened for inclusion in the study up to 28 days before dosing. Subjects were admitted to the clinical unit during Study Day −2 for Period 1 and Day −1 for Period 2 and were dosed in the morning of Day 1. On the night prior to Period 1 the subjects in the fasted/fed or fed/fasted sequence were fasted for a minimum of 10 h prior to dosing.

Subjects remained in the clinical unit and did not leave until discharged on Day 2, as per Study Schema. The minimum washout between periods was 4 days. A follow-up phone call took place 7 days after the final dose to ensure the ongoing wellbeing of the subjects.

Each subject received the following treatments (Table 4):

TABLE 4
Dosing Regimens

| | | Investigational | Route of |
|---|---|---|---|
| Sequence | Period | Medicinal Product | Administration |
| 1 | 1 | AMX0035 | Oral, Fasted |
| | 2 | | Oral, Fed (High-Fat) |
| 2 | 1 | AMX0035 | Oral Fed (High-Fat) |
| | 2 | | Oral, Fasted |

### Study Design

AMX0035 is a fixed dose combination of two small molecules, PB and TURSO, designed to block neuronal death and neurotoxic inflammation through simultaneous inhibition of ER and mitochondrial cellular stress. PB and TURSO act with unique mode of action on two independent cellular pathways.

The current study was designed to determine the plasma levels and PK parameters of PB and TURSO and major metabolites, of AMX0035 as well as to assess food effect and gender on the extent and rate of absorption. The AMX0035 formulation was expected to simultaneously target both ER stress and mitochondrial bioenergetic pathways.

### Selection of Study Population

Subjects were selected from a panel of volunteers recruited by Quotient Sciences and were screened for inclusion in the study up to 28 days before dosing.

### Inclusion Criteria

To be eligible for study entry subjects had to satisfy all of the following criteria:
1. Healthy male or female subjects aged between 40 and 65 inclusive.
2. Body mass index between 18.5 and 32.0 kg/m², inclusive.
3. Women who were not of childbearing potential by reason of surgery (bilateral tubal ligation, hysterectomy+/−oophorectomy, bilateral oophorectomy, bilateral salpingectomy), or post-menopausal (at least 12 months without menstrual period, and menopause confirmed with a follicle-stimulating hormone level of >40 IU/L at screening).
4. All subjects agreed to use one acceptable method of birth control for the duration of participation in the study and 30 days after the last dose. Acceptable methods of birth control were:
   a. Surgical sterilization of the subject (vasectomy with documented azoospermia);
   b. Use of a non-hormonal intrauterine device (IUD) for the subject's female partner with failure rate of less than 1% per year inserted by qualified physician at least one month before study drug administration and to remain in place for at least 30 days after the last dose of study drug;
   c. Barrier methods such as male condom or cap, diaphragm or sponge with spermicide,
   d. Men who were infertile (at least 3-months post-vasectomy), or truly abstinent of heterosexual intercourse, or an exclusively homosexual lifestyle were not required to use two methods of contraception but were required to commit to this lifestyle for the duration of the study and 30 days after the last dose.
5. Negative test for hepatitis B surface antigen (HBsAg), Hepatitis C antibody (HCV Ab) or human immunodeficiency virus (HIV) 1 and 2 antibodies at screening.
6. The subject had provided written informed consent prior to admission to this study.

In addition to the above criteria, subjects also agreed to the following restrictions:
No alcohol during the 48 h prior to dose administration until 24 h post-discharge.
No food or drinks containing caffeine or xanthine products (e.g. coffee, tea, cola drinks, energy-drinks and chocolate) for 24 h prior to dose administration until 24 h post-discharge.
No food or drinks containing grapefruit, grapefruit juice, Seville oranges, Seville orange marmalade, and Seville orange juice or other products containing grapefruit or Seville oranges from 7 days prior to admission, until 24 h post-discharge.
No unaccustomed strenuous exercise from the 72 h prior to dosing until 24 h post-discharge from the study.
Subjects were advised not to donate blood or plasma for at least 3 months after the last dose administration.
Subjects abided by all birth-control requirements according to their individual circumstances.

### Exclusion Criteria

Subjects were excluded from the study if one or more of the following statements was applicable:
1. Subjects with clinically significant ongoing disease or disorder, including for example: cardiovascular diseases; hypertension; cancer or neoplasia; diabetes; hepatic, endocrine, metabolic, respiratory, renal, gastrointestinal (except appendectomy) including biliary diseases or cholecystectomy, hematological or Axis I or II psychiatric disorders.
2. Clinically significant, in the opinion of the Investigator, laboratory test abnormalities at screening or at admission.
3. Clinically significant, in the opinion of the Investigator, infection or inflammation at time of screening or admission.
4. Acute gastrointestinal symptoms (e.g., nausea, vomiting, diarrhea) at time of screening or admission or a clinical diagnosis of irritable bowel syndrome (IBS) per ROME criteria.
5. Subject had an average QT interval corrected with Fridericia's formula (QTcF) >450 msec taken from screening and first admission electrocardiograms (ECGs) or any clinically significant QTcF prolongation at any time point thereafter.
6. Any current or previous illicit use of Class A drugs such as opiates, cocaine, ecstasy. LSD, and amphetamines (Class B). Subjects that admitted to occasional past use of cannabis were not excluded as long as they had a negative drugs-of abuse test at screening and admission and had been abstinent for at least 3 months.
7. An alcoholic intake greater than 14 units per week or unwillingness to stop alcohol consumption for the duration of the study. Note: 1 unit=8 g ethanol (250 mL of beer or approximately 10 oz, 1 glass wine [100 mL or approximately 3 oz], 1 measure spirits [30 mL or approximately 1 oz]).
8. Use of medication (including over-the-counter [OTC] within 14 days or 5 elimination half-lives of the medication considered. (whichever is longer) of admission.
9. Use of prescribed centrally active or psychoactive agents within 28 days from admission.
10. Use of drugs with enzyme inducing properties such as St John's Wort within 3 weeks prior to the first administration of investigational product.
11. Requirement for any medication that needed to be continued during the study.
12. Use of any investigational medication within 3 months prior to the start of this study or scheduled to receive an investigational drug during the course of this study.
13. Had participated in another clinical study within 30 days prior to screening.
14. History of plasma/blood donation in the last 2 months.
15. History of severe allergies or multiple adverse drug reactions, including penicillin and cephalosporin.
16. Any condition, which compromised ability to give informed consent or to communicate with the Investigator as required for the completion of this study.
17. Unwilling to conform to all lifestyle considerations and restrictions mandated by the protocol.
18. The subject had previously taken AMX0035 in this study.
19. Subjects who had consumed grapefruit, grapefruit juice, Seville oranges, Seville orange marmalade, and Seville orange juice or other products containing grapefruit or Seville oranges during the 7 days prior to admission.
   Removal of Subjects from the Study

If a subject wished to leave the study at any time, they were permitted to do so. Every reasonable effort was to be made by Quotient Sciences to complete a final assessment/discharge procedures. Quotient Sciences were to advise the sponsor of the withdrawal of any subject from the study.

The early withdrawal date was defined as the date of the decision to withdraw the subject from the study. The subject completion date was defined as the date of the last procedure conducted or last contact (i.e., phone call) for that subject.

If a subject requested to leave the clinical unit earlier than the planned discharge time e.g. due to unforeseen personal circumstances, but aimed to return to the unit to complete the study, this was to be documented as a subject self-discharge and a protocol deviation. The subject must have completed the planned assessments/discharge procedures before discharge from the clinical unit and were to return for the next study period/assessments, as planned.

Subjects were withdrawn from the study drug(s) for the following reasons:
Experiencing a serious or severe AE including but not limited to:
   a. Clinically significant QTc(F) of >500 msec or increase in QTc(F) of >60 msec from baseline (confirmed following a repeat ECG)
   b. Baseline was considered the closest QTcF of the triplicate ECGs prior to dosing for each period
   c. alanine aminotransferase (ALT) concentration >3×the upper limit of the reference range and total bilirubin is >2×the upper limit of the reference range
Termination of the study by the sponsor, regulatory agency or IRB
Upon the subject's request (withdrawal of consent)
Significant concurrent illness or requirement for prohibited medication
Subject non compliance
At the discretion of the investigator

For the purpose of withdrawal criteria, baseline was considered as the most recent pre-dose assessment.

For a subject who withdrew because of an IMP-related AE, every effort was to be made to ensure that the subject completed follow-up procedures. Any subject who withdrew or discontinued the study prematurely because of an IMP-related AE or termination of the study was to be considered to have completed the study, and was not to be replaced.

Early termination for any of the above reasons should be distinguished from withdrawal of consent by the subject to participate in any further activities.

Subjects withdrawing for other reasons may have been replaced at the discretion of the investigator and sponsor.

### Investigational Medicinal Products (IMP) Administered

The subject mixed and consumed the drink completely under supervision. It was normal for a small amount of powder to remain undissolved. Subjects were to consume within 60 minutes after the powder was added to water. Subjects were allowed a small amount of water to rinse (1 oz) the bitter taste of AMX0035.

The following information was recorded in the clinical database:
Date and time of administration
Confirmation that entire dose was administered

### Identity of Investigational Medicinal Product

AMX0035 was supplied by the sponsor to the site pharmacy as a carton box containing 40 single use sachets. The active pharmaceutical ingredient was TURSO and PB (AMX0035) which was supplied by the sponsor as a white powder in sachet. Each sachet contained 1 g TURSO and 3 g PB.

Authorized pharmacy personnel prepared the subject study medication by opening one sachet of AMX0035, pouring the powder into a container and adding approximately 240 mL of room temperature water. The solution was stirred until the powder was mostly dissolved.

An accountability record of utilization was maintained throughout the study. Unused IMPs were destroyed at the sponsor's request.

### Method of Assigning Subjects to Treatment Groups

On admission (Period 1), each enrolled subject was randomized to one of two sequences in a 1:1 ratio so that 7 subjects were randomized to Sequence 1 and 7 subjects were randomized to Sequence 2.
Sequence 1: AMX0035 dosed in the fasted state in Period 1 and dosed in the fed state in Period 2
Sequence 2: AMX0035 dosed in the fed state in Period 1 and dosed in the fasted state in Period 2

A treatment allocation list was produced prior to dosing using the randomization schedule.

The randomization schedule and treatment allocation list were produced according to Quotient Sciences' standard operating procedures (SOPs). The treatment allocation list provided the dosing team with information on which treatment was to be administered to each subject in each period, and was filed in the investigator site file.

### Selection of Doses in the Study

PB has been evaluated in a dose-escalating study in subjects with neurodegenerative diseases and was found to be generally safe and tolerable at significantly higher doses than assessed in this study. Specifically, the most common AEs included; falls, or other accidental injury, dizziness, diarrhea, edema, dry mouth, headache, nausea, and rash. Apart from the emergence of headaches in subjects, these AEs occurred at a higher rate compared to the placebo cohort, and are expected side effects from PB. No clinically significant changes in laboratory values, ECGs, or vital signs were observed and no deaths, unexpected or related serious AEs occurred. Importantly, these studies evaluated daily dosages of PB between 9 to 21 g and 12 to 18 g while this study used 6 g daily (Cudkowicz M E, Andres P L, Macdonald S A, et al. Phase 2 study of sodium phenylbutyrate in ALS. Amyotroph Lateral Scler. 2009; 10 (2); 99-106; Hogarth P, Lovrecic L, Krainc D. Sodium phenylbutyrate in Huntington's disease: A dose-finding study. Mov Disord. 2007; 22 (13): 1962-1964).

### Selection and Timing of Dose for Each Subject

AMX0035 was taken orally as a single dose in the morning of Day 1 in Periods 1 and 2. Any departures from the intended regimen were recorded in the eCRFs. All dose administrations were performed in the study center under the supervision of appropriately trained staff.

Meals were required to be controlled by clinical staff members on Day 1. Meals were provided at nominal times. The start and stop time of the meal was recorded in the source workbook.

For Fed Dosing

Subjects were provided with a light snack and were then fasted from all food and drink (except water) until the following morning, when they were provided with a high-fat breakfast. The breakfast was consumed over a maximum period of 25 min, and dosing occurred 30 min after the start of breakfast. Subjects were encouraged to eat their meal evenly over the 25 min period. For this trial the subject was deemed compliant if he consumed >80% of the high-fat standard breakfast.

For Fasted Dosing

The subjects in the 24-h baseline and in the fasted condition Period remained fasted up to 4 h post-dose and were offered a standardized meal to be taken between 4.00 and 5.00 h post-dose. Subjects in the fed condition were fasted overnight and received a high-fat standard breakfast 30 minutes pre-dose and then remained fasted for 4.00 to 5.00 h post-dose when they received a standardized meal.

No water was allowed (except for administration of study drug as instructed) between 1 h prior and 1 h after dose administration, except for a small volume (1 oz) rinse for taste, otherwise water was allowed ad libitum. Subjects were encouraged to hydrate regularly during the in-house stay including the fasting period but no formal volume standard was required.

### Prior and Concomitant Therapy

No medication was permitted from 14 days or 5 half-lives (whichever is longer) before IMP administration until the 7 days post-dose follow-up phone call and those deemed necessary by the investigator to treat AEs. Any medications used were recorded in the source workbook.

### Treatment Compliance

During all clinical phases of the study, subjects were observed by study staff to assure compliance to all study procedures, including dose administration.

The subject was to mix and consume the drink completely under supervision. It was normal for a small amount of powder to remain undissolved. Subjects were to consume within 60 minutes after the powder was added to water. Subjects could use a small amount of water to rinse (1 oz) the bitter taste of AMX0035.

The date and time that each subject was dosed was recorded in the subject's source as well as confirmation that the entire dose was administered. Any violation of compliance required evaluation by the investigator and sponsor to determine if the subject could continue in the study.

### Pharmacokinetic and Safety Variables

A schedule of assessments, showing the PK and safety procedures are shown in Tables 2-4, below.

When more than 1 procedure was scheduled for the same time point, the order of precedence was as follows:

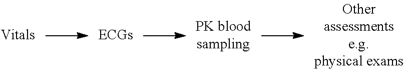

Vital signs were taken prior to ECGs when both measurements were scheduled at the same time point. Other assessments, e.g. physical examinations were performed within the required time windows. ECG and blood sample should be taken within no more than 10 minutes of each-other.

All safety assessments were timed and performed relative to the start of dosing.

TABLE 5
Schedule of Assessments: 24 h Baseline

| | | | | | | | | | | | | | | | | Also |
|---|---|---|---|---|---|---|---|---|---|---|---|---|---|---|---|---|
| | | | | | | | | | | | | | | | | Baseline |
| | | Admit | 24 h Baseline | | | | | | | | | | | | | Period 1 |
| | | to Unit | −1 | | | | | | | | | | | 1 | | 1 |
| Procedure | Screening | (1) | Time (h relative to start) | | | | | | | | | | | | | |
| Study Day | −28 to −2 | −2 | 0 | 1 | 2 | 3 | 4 | 5 | 6 | 7 | 8 | 10 | 12 | 16 | 20 | 24 |
| Explain Study/Obtain | X | | | | | | | | | | | | | | | |
| Consent | | | | | | | | | | | | | | | | |
| Demographic | X | | | | | | | | | | | | | | | |
| information | | | | | | | | | | | | | | | | |
| Medical History | X | | | | | | | | | | | | | | | |
| Inclusion/Exclusion | X | X | | | | | | | | | | | | | | |
| Screening only Labs (Serology) | X | | | | | | | | | | | | | | | |
| Safety laboratory tests | X | X | | | | | | | | | | | | | | X |
| (3) (7) | | | | | | | | | | | | | | | | |
| Urine drug and alcohol | X | X | | | | | | | | | | | | | | |
| Screen (4) | | | | | | | | | | | | | | | | |
| ECG in triplicate (5) | X | | X | X | X | X | X | X | X | X | | | X | | | X |
| Physical Examination | X | X | | | | | | | | | | | | | | |
| Vital Signs (BP, HR, | X | X | X | | X | | X | | | | | | | | | X |
| Resp. Temp) | | | | | | | | | | | | | | | | |
| Body Weight | X | X | | | | | | | | | | | | | | |
| Procedural AEs | | X | | | | | | | | | | | | | | |
| Blood Draw for PK (2) | | | X | X | X | X | X | X | X | X | X | X | X | X | X | X |
| (6) | | | | | | | | | | | | | | | | |

BP = blood pressure, HR = heart rate, Resp = respiration, Temp = temperature.
(1) Admission Day −2.
(2) Pre-study Day −1 - matched PK samples over 24 h to obtain endogenous plasma levels for TURSO and metabolites.
(3) Samples for the following safety laboratory:
Complete blood count (CBC) with red blood count, hemoglobin, hematocrit, mean cell volume (MCV), mean corpuscular hemoglobin (MCH), mean corpuscular hemoglobin concentration (MCHC), platelet count, white cell count, neutrophil count, monocyte count, lymphocyte count, eosinophil count and basophil count
Chemistry profile including serum glucose, uric acid, calcium, phosphorus, sodium, potassium, chloride, creatinine, blood urea nitrogen (BUN), aspartate aminotransferase (AST), ALT, lactate dehydrogenase (LDH), alkaline phosphatase, total bilirubin, total protein, albumin
Coagulation parameters: prothrombin time (PT) and activated partial thromboplastin time (aPTT) (Screening only)
Urinalysis (dipstick) including pH, glucose, ketones, protein and red blood cell (RBC). Discharge urine samples were taken ± 2 h from the nominal urine sampling time.
(4) Drug abuse testing including cannabinoid, cocaine, amphetamines, barbiturates and opiates. Alcohol urine test.
(5) Digital ECG matched to PK samples (to correspond to peak levels of PB and TURSO) in triplicate.
(6) PK analytics: Only TURSO and metabolites (ursodeoxycholic acid [UDCA] and glycoursodeoxycholic acid [GUDCA]) were analyzed for 24-h Baseline Period except for the 24 h time point i.e. baseline for Period 1. For Period 1 and 2 TURSO, PB and metabolites were analyzed.
(7) Follicle stimulating hormone (FSH) in all women at screening.

TABLE 6
Schedule of Assessments: Period 1

| | Period 1 | | | | | | | | | | | | | | | | | | | | | Discharge |
|---|---|---|---|---|---|---|---|---|---|---|---|---|---|---|---|---|---|---|---|---|---|---|
| | 1 | | | | | | | | | | | | | | | | | | | | | 2 |
| Procedure | Time (h relative to start) | | | | | | | | | | | | | | | | | | | | | |
| Study Day | 0 | | 0.25 | 0.5 | 0.75 | 1 | 1.5 | 2 | 2.5 | 3 | 3.5 | 4 | 4.5 | 5 | 6 | 7 | 8 | 10 | 12 | 16 | 20 | 24 |
| Safety laboratory tests (3) | Refer to | X | | | | | | | | | | | | | | | | | | | | X |
| ECG in triplicate (5) | 24 h | | | | | X | | X | | X | | X | | X | X | | | | X | | | |
| Physical Examination | baseline | | | | | | | | | | | | | | | | | | | | | X |
| Vital signs (BP, HR, Resp. | | X | | | | | | X | | | | X | | | | | | | | | | X |
| Rate, Temp) | | | | | | | | | | | | | | | | | | | | | | |
| Body weight | | | | | | | | | | | | | | | | | | | | | | X |
| Blood Draw for PK (6) | | X | X | X | X | X | X | X | X | X | X | X | X | X | X | X | X | X | X | X | X | X |
| Treatment emergent AE | | | X | | | | | | | | | | | | | | | | | | | |
| and con. meds | | | | | | | | | | | | | | | | | | | | | | |

Refer to footnote in 24 h baseline table of events (Table).

TABLE 7
Schedule of Assessments: Period 2

| | | | | | | | | | | | | | | | | | | | | | | Discharge | Follow |
|---|---|---|---|---|---|---|---|---|---|---|---|---|---|---|---|---|---|---|---|---|---|---|---|
| | Admit | | | | | | | | | | | | | | | | | | | | | or Early | up |
| | to | | | | | | | | | | | | | | | | | | | | | Termin- | (+1 |
| | Unit | Period 2 | | | | | | | | | | | | | | | | | | | | ation | day) |
| | −1 | 1 | | | | | | | | | | | | | | | | | | | | 2 | 7 |
| Procedure | Time (h relative to start) | | | | | | | | | | | | | | | | | | | | | | |
| Study Day | | 0 | 0.25 | 0.5 | 0.75 | 1 | 1.5 | 2 | 2.5 | 3 | 3.5 | 4 | 4.5 | 5 | 6 | 7 | 8 | 10 | 12 | 16 | 20 | 24 | |
| Safety laboratory | X | | | | | | | | | | | | | | | | | | | | | X | |
| tests (3) | | | | | | | | | | | | | | | | | | | | | | | |
| Urine drug and | X | | | | | | | | | | | | | | | | | | | | | | |
| alcohol screen (4) | | | | | | | | | | | | | | | | | | | | | | | |
| ECG in | | X | | | | X | | X | | X | | X | | X | X | X | | | X | | | | |
| triplicate (5) | | | | | | | | | | | | | | | | | | | | | | | |
| Physical | X | | | | | | | | | | | | | | | | | | | | | X | |
| Examination | | | | | | | | | | | | | | | | | | | | | | | |
| Vital signs | X | X | | | | | | X | | | | X | | | | | | | | | | X | |
| (BP, HR, Resp. | | | | | | | | | | | | | | | | | | | | | | | |
| Rate, Temp) | | | | | | | | | | | | | | | | | | | | | | | |
| Body weight | | | | | | | | | | | | | | | | | | | | | | X | |
| Blood Draw | | X | X | X | X | X | X | X | X | X | X | X | X | X | X | X | X | X | X | X | X | X | |
| for PK (6) | | | | | | | | | | | | | | | | | | | | | | | |
| Treatment | | X | | | | | | | | | | | | | | | | | | | | | |
| emergent AE | | | | | | | | | | | | | | | | | | | | | | | |
| and con. meds | | | | | | | | | | | | | | | | | | | | | | | |
| Phone call check | | | | | | | | | | | | | | | | | | | | | | | X |

Refer to footnote in 24 h baseline table of events (Table)

### Pharmacokinetic Measurements

### Sample Collection

Venous blood samples were withdrawn via an indwelling cannula or by venipuncture according to the time schedule presented in Table 5 Table, Table 6, and Table 7.

Blood samples of approximately 4 mL were collected into 4 mL BD Vacutainer Plastic K2EDTA Tubes with Lavender Hemogard Closure, and was gently inverted approximately 8 times. The tubes were then immediately stored on ice and processed within 60 min of collection by centrifugation at 1500 g for 10 min at 2 to 8° C. The resultant plasma was separated equally into 2 aliquots and transferred to 2 mL appropriately labelled primary and backup polypropylene cryovials. Samples were stored at −20° C. or below until they were shipped to WWCT for the analysis of AMX0035.

### Analytical Method

The plasma concentrations of sodium PB, PAA, TURSO, ursodeoxycholic acid (UDCA) and glycoursodeoxycholic acid (GUDCA) were determined using validated analytical methods at Worldwide Clinical. The lower limit of quantification was 1.6 μg/mL sodium PB, 0.8 μg/mL for PAA, 0.02 μg/mL for TURSO, and 20 ng/mL for UDCA and GUDCA.

### Safety Measurements

Adverse Events

An AE was any untoward medical occurrence in a subject administered a pharmaceutical product, which did not necessarily have a causal relationship with this treatment. An AE could be any unfavorable or unintended sign (including an abnormal laboratory finding), symptom or disease temporally associated with the administration of an IMP, whether or not considered related to the IMP. Pre-existing conditions that worsened during the study were to be reported as AEs. An adverse drug reaction (ADR) is any AE where a causal relationship with the IMP is at least a reasonable possibility (possibly related or related).

Any clinically significant abnormality in laboratory parameters, vital signs or ECG could have been reported as an AE according to the judgement of the PI, taking into account any associated clinical signs and symptoms and pre-dose values.

All AEs were recorded from the time of providing written informed consent until 30 days after the last dose of study drug. During each study visit the subject was questioned and/or examined by the investigator or his/her designee for evidence of AEs. Each AE was recorded on the subject's source workbook, stating the date and time of onset, a description of the AE, duration, seriousness (yes/no), severity (mild [Grade 1], moderate [Grade 2], severe [Grade 3], very severe or life threatening [Grade 4]), action taken, outcome, and an Investigator's opinion on the relationship between the study treatment and the event. A diagnosis and final opinion on the relationship (unrelated, possibly related or related) between the study treatment and the event was provided at the end of the study by the Investigator.

Subjects withdrawn from the study due to an AE were followed up until the outcome was determined and written reports were provided by the Investigator.

### Serious Adverse Events

A serious adverse event was any untoward medical occurrence or effect that, at any dose, resulted in death, was life-threatening, required or prolonged inpatient hospitalization, resulted in persistent or significant disability/incapacity, was a congenital anomaly/birth defect or was considered an important medical event as recognized by the PI.

A suspected unexpected serious adverse reaction (SUSAR) was any unintended response to an IMP related to any dose, i.e. a causal relationship between the IMP and the AE was at least a reasonable possibility, that was both serious and not consistent with the applicable product information. All SUSARs were the subject of expedited reporting.

### Laboratory Parameters

The following laboratory assessments were performed at the time points specified in Tables 2, 3, and 4.

Hematology: blood samples were collected into K2EDTA-coated tubes (4 mL) with Lavender Hemogard Closure. The following analyses were performed: hemoglobin, hematocrit, erythrocytes, erythrocyte distribution width, erythrocyte mean corpuscular volume, erythrocyte mean corpuscular hemoglobin, erythrocyte mean corpuscular hemoglobin concentration, platelet count, mean platelet volume, leukocytes, neutrophils, lymphocytes, monocytes, eosinophils and basophils.

Clinical Chemistry: blood samples were collected into serum separator tubes (7.5 mL) containing clot activator and serum gel separator. The following analyses were performed: sodium, potassium, chloride, blood urea nitrogen, urate, creatinine, total bilirubin, direct bilirubin, alkaline phosphatase, aspartate aminotransferase, ALT, total protein, albumin, lactate dehydrogenase, calcium, phosphate, glucose.

Coagulation: blood samples were collected into sodium citrate tubes (2.7 mL). The following analyses were performed: prothrombin time (PT), prothrombin international normalized ratio and activated partial thromboplastin time (aPTT).

Urinalysis: the following analyses were performed on urine samples (>20 mL) using urine test strips: pH, specific gravity, glucose, ketones, nitrite, leukocyte esterase, protein, urobilinogen, blood and bilirubin.

Drug Screen: urine samples (>20 mL) were tested for drugs, including drugs of abuse, at screening and admission.

Virology: screening for HBsAg, HCV Ab and HIV (1 and 2) was performed using the clinical chemistry blood sample.

Alcohol Tests: these were performed at screening and admission for each study period.

In cases where laboratory findings were outside the normal range and the Investigator believed that the results may have been of clinical significance, repeat sampling was requested except in the case of positive virology results. If the abnormal finding was clinically significant, appropriate actions were taken e.g. the subject was not entered into the study or the subject was withdrawn from the study. The subject was referred to their general practitioner or other appropriate provider (e.g. genitourinary medicine clinic) for further care. The same applied if the results of the HBsAg, HCV Ab or HIV test were positive and the Investigator ensured that adequate counselling was available if requested.

Abnormal findings at follow-up assessments also required repeat testing if the Investigator believed the results may have been of clinical significance.

### Vital Signs

Vital signs (systolic and diastolic blood pressure [BP], heart rate, and oral temperature) were measured by an automated recorder after the subject had been in a supine position for a minimum of 5 min using the arm opposite to blood draws according to the time schedule presented in Tables 2, 3, and 4. BP and pulse measurements were to be preceded by 5 minutes of rest for the subject.
The pre-dose vital signs measurements were taken ≤2 h before dosing.
Post-dose vital signs measurements were taken ±15 min from the nominal post-dose time points.
Discharge vital signs measurements were taken ±1 h from the nominal time point.
For return visits vital signs measurements were taken ±2 h from the nominal return visit time point.

### Electrocardiogram

Digital 12-lead ECGs were recorded after the subject had been in the supine position for a minimum of 15 min as detailed in Tables 2, 3, and 4. Triplicate ECGs were to be taken one-to-two minutes apart.

ECGs were evaluated for QT. QTcF, PR, QRS, RR and HR intervals and the data collected in the clinical database. ECGs were interpreted by the investigators at Quotient.
The pre-dose ECG measurements were taken ≤2 h before dosing.
Post-dose ECG measurements were taken ±15 min from the nominal post-dose time point.
Discharge ECG measurements were taken ±1 h from the nominal time point.
For return visits ECG measurements were taken ±2 h from the nominal return visit time point.

### Physical Examination

Subjects underwent a complete physical examination, including a neurological exam, as detailed in Tables 2, 3, and 4. Any changes were recorded. Height was collected once and weight was measured as per the Schedule of assessments.

### Pregnancy

Male subjects agreed to notify the PI if their partner became pregnant during the study. Any pregnancy was to be followed and the status of the mother and child was to be reported to the sponsor after delivery where possible.

### Appropriateness of Measurements

The nature and timing of the safety assessments as detailed in the final protocol were considered appropriate to assess the safety of active drug taking into account the nature of the compound and its route of administration.

The timing of the blood samples and urine collection intervals was considered appropriate to assess the PK profile of the active drug and metabolites.

### Data Quality Assurance

All study data recorded in the source workbook were transcribed into a validated database (InForm v5.0). Quality control, data validation and query resolution were carried out in accordance with Quotient Sciences SOPs. All source documents produced during the study were made available by the PI for review and source data verification by representatives of the sponsor, regulatory authorities and appropriate IRB.

### Statistical Methods and Determination of Sample Size

Analysis Populations

The safety population included all subjects who received any amount of IMP.

The safety analysis set was to be defined on a treatment basis and included all relevant data from the subjects included in the safety population who had received that treatment. For the purposes of subject disposition, demographic and baseline tables, the safety analysis set was also to be defined on a sequence basis (i.e. all subjects randomized to that sequence who received at least one treatment).

The PK population was to include all subjects who received at least 1 dose of IMP and who had no missing samples or invalid post-dose analytical results at critical time points e.g. around the Cmax, no relevant protocol deviations (e.g. subjects were deemed compliant if they consumed >80% of the high fat standard breakfast) that may have impacted the study objectives with respect to the PK endpoints, and no relevant AEs such as vomiting that suggested that the whole dose was not absorbed for a particular subject.

The PK analysis set was to be a subset of the PK population and was to be defined on a treatment basis and included all relevant data from the subjects included in the PK population who received that treatment. Individual subject profiles (i.e. periods) were to be excluded from the PK analysis set where deemed appropriate, such as, if the subject in the study period affected did not meet the criteria above or other study emergent point related to PK analysis or interpretation.

If required, a PK analysis subset was to be determined at the same time as the PK population and was based on the PK analysis set, if additional subjects were required to be excluded from the statistical analysis (see Section “Populations Analyzed”).

The safety-PK population/analysis set was to include all subjects who were included in both the safety and PK population/analysis set.

Pharmacokinetic Analysis

The PK parameters for PB and metabolite (phenylacetate [PAA]), TURSO and metabolites (UDCA and GUDCA) in plasma were to be estimated where possible and appropriate for each enrolled subject and period by non-compartmental analysis methods using Phoenix® WinNonlin software (v8.0 Certara USA. Inc., USA) and/or SAS® version 9.4 (SAS Institute. Inc., Cary. North Carolina. USA). Actual elapsed time from dosing was to be used for the final plasma PK parameter calculations after database lock.

The PK parameters for PB and PAA were to be derived from actual concentrations, i.e. non-baseline corrected concentrations.

Prior to PK parameter derivation of TURSO and metabolites (UDCA and GUDCA), plasma concentrations were to be baseline corrected for each treatment period using time matched baseline concentrations from Day −1, Period 1 in SAS (version 9.4).

The PK parameters were not to be determined on non-baseline corrected concentrations of TURSO and metabolites (UDCA and GUDCA).

The following parameter estimates were to be estimated where possible:
Tmax: Time of maximum observed concentration
Cmax: Maximum observed concentration
AUC(0-last): Area under the curve from 0 time to last measurable concentration
AUC(0-inf): Area under the curve from 0 time extrapolated to infinity
AUCextrap: Percentage of AUC(0-inf) extrapolated beyond the last measurable concentration
T1/2: Apparent elimination half-life
lambda-z: Slope of the apparent elimination phase
CUJF: Apparent plasma clearance calculated after a single oral administration where F (fraction of dose absorbed) is unknown (parent only)
Vz/F: Apparent volume of distribution based on the terminal phase calculated after a single extravascular administration where F (fraction of dose absorbed) is unknown (parent only)
Frel Cmax: Relative bioavailability based on Cmax
Frel AUC(0-last): Relative bioavailability based on AUC(t-last)
Frel AUC(0-inf): Relative bioavailability based on AUC(0-inf)
MR Cmax: Metabolite to parent ratio based on Cmax
MR AUC(0-last): Metabolite to parent ratio based on AUC(0-last)
MR AUC(0-inf): Metabolite to parent ratio based on AUC(0-inf)

Summary statistics (i.e. n, mean, standard deviation [SD], coefficient of variation [CV %], median, minimum, maximum, geometric n, geometric mean, geometric SD and geometric CV %) were to be calculated for plasma concentrations by treatment (i.e. food status) and time point, where possible.

Summary statistics (i.e. n, mean. SD, CV %, median, minimum, maximum) were to be calculated for all plasma PK parameters using the PK analysis set/subset by treatment (i.e. food status). Geometric n, geometric mean, geometric SD and geometric CV % were presented for all PK parameters (except Tmax).

Summary statistics of plasma concentrations were to be calculated and presented for the following:
PB and PAA: actual concentrations
TURSO, UDCA and GUDCA: actual concentrations (including Day −1) and time matched baseline corrected concentrations

Additional summaries of plasma concentrations were to be presented by gender.

Arithmetic mean plasma concentration vs time curves were to be produced by treatment on a linear/linear scale and error bars for ±arithmetic SD was included on the plots.

Geometric mean plasma concentration vs time curves were to be produced by treatment on a log₁₀/linear scale. Error bars were included on these plots, where the error bars are (geometric mean×/÷geometric SD).

Spaghetti plots by analyte and treatment were to display one line per profile per subject using actual sampling time after dosing. The plots were to be produced on both a linear/linear and a log₁₀/linear scale. A legend identifying individual subject profiles was to be displayed on the plots.

Individual Cmax, AUC(0-last), AUC(0-inf) and Tmax values were to be plotted for each individual within the relevant PK analysis dataset on a linear/linear scale, i.e. fed and fasted regimens will represent the x-axis. Each subject's individual points were connected through a line. Separate plots were to be provided for each analyte.

### Statistical Analysis of Pharmacokinetic Parameters

Food Effect Assessment: Cmax, AUC(0-last) and AUC(0-inf)

Statistical analysis was to be performed on the PK parameters Cmax, AUC(0-last) and AUC(0-inf) for PB and PAA (based on actual concentrations) and TURSO, UDCA and GUDCA (based on time matched baseline corrected concentrations) to assess the presence of a food effect. The null hypothesis to be tested was that there was no difference between fed and fasted treatments.

The PK parameters were to undergo a natural logarithmic transformation and were to be analyzed using mixed effect modelling techniques. The full model was to include terms for treatment (i.e. fed or fasted), period, gender, treatment by gender interaction and sequence fitted as fixed effects and subject within sequence as a random effect

If the treatment by gender interaction term was not significant at the 5% level (i.e. p>0.05), then this term was dropped from the model, and a reduced model was to be used and only results for both genders combined were to be presented. If the interaction term was significant at the 5% level (i.e. p≤0.05), the results were to be presented for both genders combined and separately for males and females.

All other statistical tests relating to PK parameters were to be 2-sided and were to be performed using a 10% significance level, leading to 90% (2-sided) CIs. The adjusted means, including the differences for the fasted/fed comparison and the associated 90% confidence intervals (CIs) obtained from the model, were to be back transformed on the log scale to obtain adjusted geometric mean ratios (GMRs) and 90% CIs of the ratio. These were to be presented together with the p-value from the fasted/fed comparison and the intra-subject variability values (denoted as CVw in the results table).

The statistical analysis was to be performed using actual treatment received and planned sequence as detailed on the randomization schedule. The model was to be fitted using the SAS Software procedure PROC MIXED, the method was to be specified as Restricted Maximum Likelihood and the denominator degrees of freedom for the fixed effects were to be calculated using Kenward and Roger's method.

Food Effect Assessment: Tmax

Comparisons of Tmax values between treatment groups were to be investigated using non-parametric analysis. Period differences (Period 1 minus Period 2) were to be derived for each subject. Hodges-Lehmann estimation methods were to be used to estimate the median difference in Tmax between treatments. The associated 90% CI and p-value were also to be derived. The null hypothesis was that the difference between the treatment groups' medians is equal to zero. The alternative hypothesis was that the difference between the treatment groups' medians was not equal to zero. A 90% CI which did not contain zero, supported by a p-value <0.10 would be an indication of a statistically significant difference between the treatments' medians with respect to Tmax.

This procedure was to be performed in SAS Software procedure PROC NPAR1WAY.

The median of the differences together with the associated 90% CI and p-value were to be presented along with the median Tmax value and n for both treatments.

Safety Parameters

The evaluation of safety parameters was to comprise analysis of AEs, laboratory variables, vital signs, ECG and physical examination findings. AEs and medications were to be coded using the Medical Dictionary for Regulatory Activities (MedDRA; v 22.0) and the World Health Organization Drug Dictionary Enhanced (2019 Q1), respectively.

Treatment-emergent AEs (TEAEs) (i.e. those beginning after dosing with study drug) were to be summarized for each treatment by system organ class (SOC) and within a SOC by the type of event, by severity and by relationship to IMP. If the severity or IMP relationship of an AE was missing, the severity/relationship was to be tabulated as missing in the summary tables. TEAEs and pre-dose AEs are listed.

Laboratory parameters, including changes from baseline, were to be summarized for each scheduled time point by treatment. Shift tables (from baseline to each post-baseline time point) are also presented.

Vital signs and ECG data, including changes from baseline, were to be summarized for each scheduled time point for each study part. The numbers of subjects with ‘substantial’ increases or decreases from baseline in systolic BP (>20 mmHg), diastolic BP (>10 mmHg) and HR (>15 bpm) were to be summarized. The number and percentage of subjects with the following ECG characteristics were also to be summarized: QTcF of ≤450 msec, 451 to 480 msec, 481 to 500 msec, >500 msec; increases in QTcF interval from baseline of <30 msec, 30 to 60 msec and >60 msec; and QT interval of ≤500 msec and >500 msec.

All laboratory parameters, vital signs, ECG data and abnormal physical examination findings were to be listed for each time point; where appropriate, values outside the reference ranges were to be flagged.

Statistical Analysis of ECG Data (Exposure Response Analysis)

The exposure-response analysis was to be performed using a combined safety-PK analysis set.

Exposure-response analysis was to be performed to assess the relationship between the time matched baseline corrected QTcF (ΔQTcF) values and the plasma concentrations for each of the parent drug and associated metabolites.

The time matched change from baseline (i.e. ΔQTcF) based on the mean of triplicate values was to be calculated for each subject at each post-dose time point, as follows:

ΔQTcF=(QTcF at relevant post baseline time point)−(QTcF at relevant baseline time point)

All concentrations below the limit of quantification concentrations were to be imputed as zero.

The relationship between ΔQTcF and AMX0035 plasma concentrations for each analyte (i.e., PB, PAA, TURSO, UDCA and GUDCA) was to be analyzed using a linear mixed model with plasma concentration for each analyte fitted as a covariate, time matched baseline QTcF as a continuous covariate and ΔQTcF as a dependent variable. Subject-specific random effects were to be added on intercept and slope parameters with an unstructured covariance matrix. If the unstructured covariance matrix was not supported by the data, other simplified or reduced structures were to be investigated (e.g., variance components).

The models were to be fitted using PROC MIXED in SAS, the method was to be specified as Restricted Maximum Likelihood and the denominator degrees of freedom for the fixed effects were to be calculated using Kenward and Roger's method.

The model was originally to be fitted with all analytes. The model was then to be fitted iteratively, removing the analyte with the least significant slope (i.e. largest p-value associated with a null hypothesis that slope=0 on a per analyte basis) for the model at each step until only a single analyte remains. As a result, a total of 5 models were to be fitted where 5 is the number of analytes. Using standard PROC MIXED model fit diagnostics, the model with the best fit was to be selected as primary, i.e. the model with the smallest Akaike Information Criterion (AIC).

Using the primary model fit (i.e. best fit), the median Tmax was to be identified for each analyte included in the model. The geometric mean concentration value for each analyte at the corresponding Tmax was to be identified and used to estimate the effect on ΔQTcF (90% CI), i.e. if 3 analytes are included in the model, then the effect on ΔQTcF was to be determined using 3 sets of data based on the geometric mean concentration corresponding to Tmax for Analyte 1, similarly for Tmax for Analyte 2 and Tmax for Analyte 3. To conclude that there is no significant effect of AMX0035 on changes in QTcF, the upper limit of the two-sided 90% CI the predicted mean change should be below 10 msec for each geometric mean concentration (i.e. one per analyte) as described above.

### Changes in the Conduct of the Study or Planned Analyses

Changes in the Planned Analyses

Due to the 24 h baseline (Day −1) ECG monitoring being conducted in the fasted state (i.e. a high fat breakfast was not provided at the time matched time of dosing on Day −1), the baseline was not truly representative of the fed state while naïve to drug. Therefore, the exposure response analysis was performed on two occasions: once using only fasted data, which was the primary analysis, and again using only fed data as an exploratory analysis.

### Study Subjects

Fourteen subjects were enrolled in this study with 7 subjects each randomized to either the fasted/fed or the fed/fasted treatment sequence. All 14 subjects were dosed and 13 (92.9%) subjects completed the study. One (14.3%) subject was discontinued from the fed/fasted treatment sequence at the discretion of the investigator.

Protocol Deviations

The study was conducted in accordance with the clinical protocol with no major protocol deviations, a minor deviation from the protocol occurred (missed or late safety or laboratory assessment/third ECG in triplicate ECG was inadvertently missed during the 3 h time point of Period 1 Day −1; data not shown).

It was the opinion of the PI and the sponsor that the protocol deviation did not affect the overall integrity or quality of the study results or lead to a safety issue for any subject.

All subjects signed a study-specific ICF before any study procedures were performed and met the inclusion and exclusion criteria; data not shown.

Populations Analyzed

Overall, all 14 subjects received at least 1 dose of IMP; therefore, all 14 subjects were included in the safety population.

All 14 subjects received at least 1 dose of IMP and had no missing samples, invalid post-dose analytical results, relevant protocol deviations or AEs that may have affected any PK endpoints for at least 1 profile (see Section “Analysis Populations”). Therefore, all 14 subjects were included in the PK population.

The subjects included in the safety. PK, and the safety-PK analysis sets for each treatment were as follows:
AMX0035 in the fed state consisted of all 14 subjects
AMX0035 in the fasted state consisted of only 13 subjects. One subject withdrew between Period 1 and 2 due to significant concurrent illness or requirement for prohibited medication (TEAE of musculoskeletal pain that led to withdrawal of IMP) and therefore received administration of AMX0035 in the fed state only.

Finally, 13 subjects were included in the PK analysis subset; one subject was excluded from this subset because he did not complete the fasted treatment.

Demographic and Other Baseline Characteristics

Demographic Variables

Individual subject demographic and lifestyle details are not provided, a brief summary is provided below.

Fourteen healthy male (8 [57.1%]) and female (6 [42.9%]) subjects between 45 and 64 years of age were entered into the study (Table 8). Most of the subjects were white (12 [85.7%]) and 2 (14.3%) subjects were black or African American. All subjects were of Hispanic or Latino ethnicity. All subjects were within the reference range for body mass index as required by the protocol (18.5 to 32.0 kg/m²). All 14 subjects were non-smokers and none were consumers of alcohol.

TABLE 8
Demographic Characteristics: Safety Analysis Set

| | | Treatment Sequence | | |
|---|---|---|---|---|
| | | Fasted/Fed | Fed/Fasted | OVERALL |
| | | (N = 7) | (N = 7) | (N = 14) |
| | | n (%) | n (%) | n (%) |
| Age (years) | n | 7 | 7 | 14 |
| | Mean | 52.7 | 54.6 | 53.6 |
| | SD | 6.8 | 5.5 | 6.0 |
| | Median | 52.0 | 54.0 | 53.0 |
| | Min | 45 | 47 | 45 |
| | Max | 63 | 64 | 64 |
| Ethnicity n | HISPANIC OR LATINO | 7 (100) | 7 (100) | 14 (100) |
| (%) | NOT HISPANIC OR | 0 | 0 | 0 |
| | LATINO | | | |
| Race n (%) | AMERICAN INDIAN OR | 0 | 0 | 0 |
| | ALASKA NATIVE | | | |
| | ASIAN | 0 | 0 | 0 |
| | BLACK OR AFRICAN | 2 (28.6) | 0 | 2 (14.3) |
| | AMERICAN | | | |
| | NATIVE HAWAIIAN OR | 0 | 0 | 0 |
| | OTHER PACIFIC | | | |
| | ISLANDER | | | |
| | WHITE | 5 (71.4) | 7 (100) | 12 (85.7) |
| | OTHER | 0 | 0 | 0 |
| Sex n (%) | Male | 4 (57.1) | 4 (57.1) | 8 (57.1) |
| | Female | 3 (42.9) | 3 (42.9) | 6 (42.9) |
| Height (cm) | n | 7 | 7 | 14 |
| | Mean | 170.2 | 164.3 | 167.3 |
| | SD | 13.3 | 4.6 | 10.1 |
| | Median | 168.0 | 164.5 | 166.3 |
| | Min | 155 | 157 | 155 |
| | Max | 193 | 170 | 193 |
| Weight (kg) | n | 7 | 7 | 14 |
| | Mean | 81.51 | 75.73 | 78.62 |
| | SD | 15.20 | 4.94 | 11.27 |
| | Median | 75.20 | 74.50 | 74.85 |
| | Min | 65.8 | 68.7 | 65.8 |
| | Max | 108.8 | 81.4 | 108.8 |
| BMI (kg/m²) | n | 7 | 7 | 14 |
| | Mean | 28.03 | 28.11 | 28.07 |
| | SD | 2.98 | 2.24 | 2.54 |
| | Median | 29.20 | 27.60 | 28.40 |
| | Min | 23.3 | 24.1 | 23.3 |
| | Max | 31.5 | 31.0 | 31.5 |

There were no clinically significant differences between treatment sequences with respect to demographic characteristics, however, the fasted/fed sequence had 2 (28.6%) subjects that were black or African American, unlike the fed/fasted sequence which had white subjects only.

Other Baseline Characteristics

Individual subjects' medical and surgical history details did not prevent enrolment in the study.

No subjects reported taking medications prior to dosing.

One subject reported a pre-dose AE of skin abrasion on the left foot and the upper arm. The event resolved without the need for medication.

All urine drug screen, urine alcohol screen and serum pregnancy tests were negative. Serology tests were non-reactive and no coagulation test results were outside of the reference ranges. As per the protocol, all subjects were post-menopausal (FSH >40 IU/L) except for one subject, who had bilateral tubal ligation and thus was of non-childbearing potential.

Measurements of Treatment Compliance

During all clinical phases of the study, subjects were observed by study staff to assure compliance to all study procedures, including dose administration.

The date and time that each subject was dosed was recorded in the subject's source workbook. Individual subjects' dosing details and subjects' compliance with meal requirements were all recorded but not provided herein.

### Pharmacokinetic Evaluation

Pharmacokinetics of PB and PAA

PB

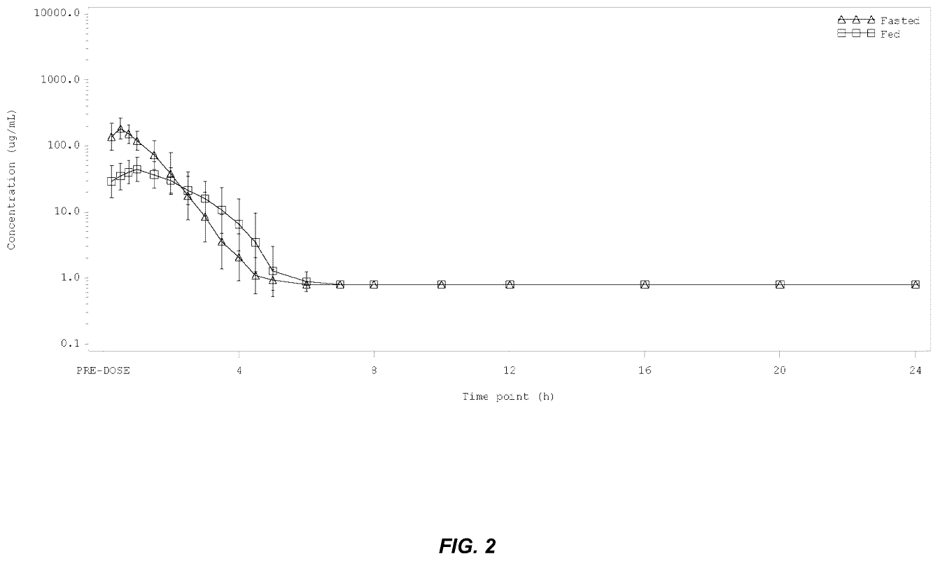

The mean plasma concentration vs time profiles for PB are illustrated by food condition on a log₁₀/linear scale for all subjects in the PK analysis set in FIG. 2.

Key PK parameters for PB for all subjects in the PK analysis set are presented below in Table 9.

TABLE 9
Summary of Plasma Pharmacokinetic Parameters for PB Following Single Oral Doses of AMX0035 to Healthy Male and Female
Subjects in the Fasted and Fed States: Pharmacokinetic Analysis Set

| | | Tmax | Cmax | AUC(0-last) | AUC(0-inf) | AUCextrap | T1/2 | Lambda-z |
|---|---|---|---|---|---|---|---|---|
| Treatment | Statistic | (h) | (ug/mL) | (ug · h/mL) | (ug · h/mL) | (%) | (h) | (1/h) |
| AMX0035 | n | 13 | 13 | 13 | 13 | 13 | 13 | 13 |
| FASTED | Median | 0.500 | 209 | 254 | 256 | 0.749 | 0.438 | 1.5829 |
| (N = 13) | Min | 0.25 | 64.4 | 73.5 | 74.8 | 0.30 | 0.40 | 0.9728 |
| | Max | 0.50 | 260 | 423 | 425 | 1.62 | 0.71 | 1.7321 |
| | Geometric n | NC | 13 | 13 | 13 | 13 | 13 | 13 |
| | Geometric Mean | NC | 188 | 236 | 237 | 0.628 | 0.461 | 1.5051 |
| | Geometric CV % | NC | 39.0 | 45.2 | 44.9 | 51.0 | 15.1 | 15.1 |
| AMX0035 | n | 14 | 14 | 14 | 13 | 13 | 13 | 13 |
| FED | Median | 1.000 | 52.7 | 120 | 126 | 1.772 | 0.560 | 1.2372 |
| (N = 14) | Min | 0.25 | 12.9 | 34.6 | 76.2 | 0.90 | 0.36 | 0.6166 |
| | Max | 1.50 | 70.5 | 198 | 200 | 6.93 | 1.12 | 1.9284 |
| | Geometric n | NC | 14 | 14 | 13 | 13 | 13 | 13 |
| | Geometric Mean | NC | 46.8 | 109 | 122 | 2.231 | 0.599 | 1.1579 |
| | Geometric CV % | NC | 45.4 | 45.4 | 28.6 | 70.7 | 41.4 | 41.4 |

| | | CL/F | Vz/F | Frel Cmax | Frel AUC(0-last) | Frel AUC(0-inf) |
|---|---|---|---|---|---|---|
| Treatment | Statistic | (mL/min) | (L) | (%) | (%) | (%) |
| AMX0035 | n | 13 | 13 | NC | NC | NC |
| FASTED | Median | 195 | 7.85 | NC | NC | NC |
| (N = 13) | Min | 118 | 4.79 | NC | NC | NC |
| | Max | 669 | 24.4 | NC | NC | NC |
| | Geometric n | 13 | 13 | NC | NC | NC |
| | Geometric Mean | 211 | 8.40 | NC | NC | NC |
| | Geometric CV % | 44.9 | 45.8 | NC | NC | NC |
| AMX0035 | n | 13 | 13 | 13 | 13 | 12 |
| FED | Median | 397 | 17.9 | 25.327 | 46.806 | 46.546 |
| (N = 14) | Min | 250 | 9.51 | 19.04 | 32.37 | 34.46 |
| | Max | 656 | 60.3 | 33.73 | 65.46 | 65.96 |
| | Geometric n | 13 | 13 | 13 | 13 | 12 |
| | Geometric Mean | 410 | 21.3 | 24.259 | 45.124 | 45.982 |
| | Geometric CV % | 28.6 | 67.0 | 18.1 | 19.9 | 19.3 |

Each treatment comprised one sachet of AMX0035, which contains 1 g TURSO and 3 g sodium PB. NC = Not Calculated.

Following a single oral administration of AMX0035 to healthy male and female subjects in the fasted state, concentrations of PB were evident from 0.25 h in all 13 dosed subjects. Maximum plasma concentrations occurred between 0.25 and 0.50 h post-dose. Concentrations then declined in a generally monophasic manner and remained quantifiable until between 3.00 and 5.00 h post-dose. Terminal slopes were reliably determined for all 13 subjects and resultant elimination half-lives ranged between 0.40 and 0.71 h. The geometric mean (geometric CV %) half-life was 0.461 h (15.1%).

The geometric mean (geometric CV %) apparent volume of distribution and apparent plasma clearance were 8.4 L (45.8%) and 211 mL/min (44.9%), respectively.

Following a single oral administration of AMX0035 in the fed state, concentrations of PB were evident from 0.25 h in all 14 subjects. Maximum plasma concentrations occurred between 0.25 and 1.50 h post-dose. Concentrations then declined in a generally monophasic manner and remained quantifiable until between 3.50 and 6.00 h post-dose. Terminal slopes were reliably determined for 13 subjects and resultant elimination half-lives ranged between 0.36 and 1.12 h. The geometric mean (geometric CV %) half-life was 0.599 h (41.4%). Where terminal slopes could not be reliably determined, this was a result of an unacceptable coefficient of determination (i.e. adjusted R²<0.9). One subject showed decreased exposure (in terms of Cmax, AUC(0-last) and AUC(0-inf) to PB compared to the rest of the group, following administration of AMX0035 in both the fasted and fed states.

The geometric mean (geometric CV %) apparent volume of distribution and apparent plasma clearance were 21.3 L (67.0%) and 410 mL/min (28.6%), respectively.

PAA

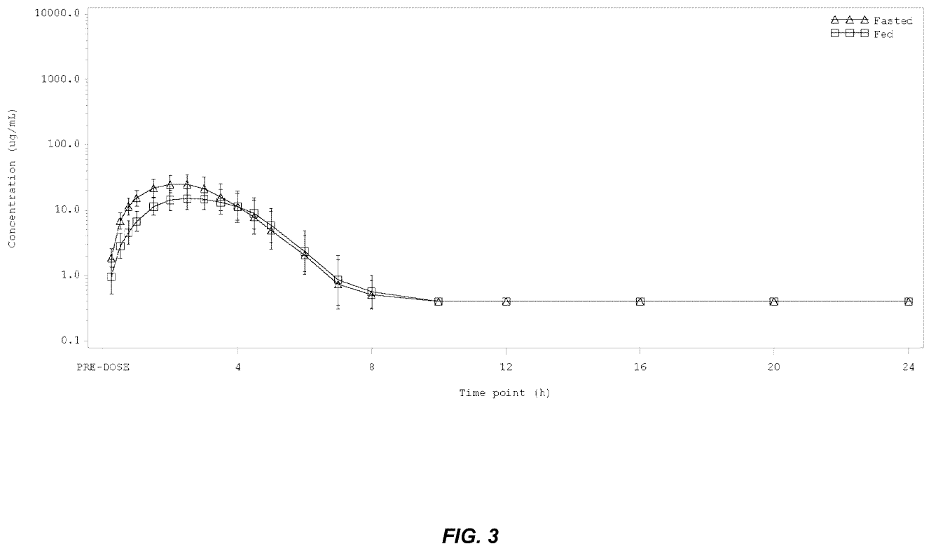

The mean plasma concentration vs time profiles for PAA are illustrated by food condition on a log₁₀/linear scale for all subjects in the PK analysis set in FIG. 3.

Key PK parameters for PAA for all subjects in the PK analysis set are presented below in Table 10.

TABLE 10
Summary of Plasma Pharmacokinetic Parameters for PAA Following Single Oral Doses of AMX0035 to Healthy Male and
Female Subjects in the Fasted and Fed States: Pharmacokinetic Analysis Set

| | | Tmax | Cmax | AUC(0-last) | AUC(0-inf) | AUCextrap | T1/2 | Lambda-z |
|---|---|---|---|---|---|---|---|---|
| Treatment | Statistic | (h) | (ug/mL) | (ug · h/mL) | (ug · h/mL) | (%) | (h) | (1/h) |
| AMX0035 | n | 13 | 13 | 13 | 13 | 13 | 13 | 13 |
| FASTED | Median | 2.500 | 27.7 | 79.3 | 80.7 | 1.963 | 0.834 | 0.8310 |
| (N = 13) | Min | 1.50 | 13.3 | 43.8 | 45.2 | 1.06 | 0.69 | 0.7287 |
| | Max | 3.50 | 42.3 | 141 | 142 | 3.18 | 0.95 | 0.9999 |
| | Geometric n | NC | 13 | 13 | 13 | 13 | 13 | 13 |
| | Geometric Mean | NC | 26.4 | 81.8 | 83.4 | 1.779 | 0.813 | 0.8527 |
| | Geometric CV % | NC | 30.7 | 38.0 | 37.4 | 36.1 | 11.5 | 11.5 |
| AMX0035 | n | 14 | 14 | 14 | 14 | 14 | 14 | 14 |
| FED | Median | 2.500 | 16.0 | 53.1 | 54.5 | 2.680 | 0.784 | 0.8899 |
| (N = 14) | Min | 2.00 | 8.39 | 30.6 | 31.9 | 1.26 | 0.60 | 0.6696 |
| | Max | 4.50 | 36.5 | 128 | 130 | 4.13 | 1.04 | 1.1486 |
| | Geometric n | NC | 14 | 14 | 14 | 14 | 14 | 14 |
| | Geometric Mean | NC | 15.9 | 56.7 | 58.3 | 2.485 | 0.780 | 0.8886 |
| | Geometric CV % | NC | 39.1 | 43.0 | 42.1 | 42.8 | 16.3 | 16.3 |

| | | Frel Cmax | Frel AUC(0-last) | Frel AUC(0-inf) | | MR | MR |
|---|---|---|---|---|---|---|---|
| Treatment | Statistic | (%) | (%) | (%) | MR Cmax | AUC(0-last) | AUC(0-inf) |
| AMX0035 | n | NC | NC | NC | 13 | 13 | 13 |
| FASTED | Median | NC | NC | NC | 0.153 | 0.398 | 0.405 |
| (N = 13) | Min | NC | NC | NC | 0.09 | 0.21 | 0.21 |
| | Max | NC | NC | NC | 0.38 | 0.77 | 0.78 |
| | Geometric n | NC | NC | NC | 13 | 13 | 13 |
| | Geometric Mean | NC | NC | NC | 0.168 | 0.416 | 0.421 |
| | Geometric CV % | NC | NC | NC | 40.3 | 38.9 | 38.5 |
| AMX0035 | n | 13 | 13 | 13 | 14 | 14 | 13 |
| FED | Median | 58.065 | 69.952 | 70.563 | 0.408 | 0.671 | 0.614 |
| (N = 14) | Min | 45.73 | 57.31 | 58.16 | 0.17 | 0.22 | 0.23 |
| | Max | 86.29 | 91.27 | 91.45 | 0.97 | 1.15 | 1.15 |
| | Geometric n | 13 | 13 | 13 | 14 | 14 | 13 |
| | Geometric Mean | 60.480 | 70.024 | 70.606 | 0.407 | 0.626 | 0.598 |
| | Geometric CV % | 20.6 | 15.4 | 15.0 | 45.6 | 49.2 | 46.8 |

Each treatment comprised one sachet of AMX0035, which contains 1 g TURSO and 3 g sodium PB. NC = Not Calculated.

Following a single oral administration of AMX0035 to healthy male and female subjects in the fasted state, concentrations of metabolite PAA were evident from 0.25 h in all 13 dosed subjects. Maximum plasma concentrations occurred between 1.50 and 3.50 h post-dose. Concentrations then declined in a monophasic/biphasic manner and remained quantifiable until between 6.00 and 8.00 h post-dose. Terminal slopes were reliably determined for all 13 subjects and resultant elimination half-lives ranged between 0.69 and 0.95 h. The geometric mean (geometric CV %) half-life was 0.813 h (11.5%).

The geometric mean (geometric CV %) PAA/PB metabolite to parent ratios following administration in the fasted state were 0.168 (40.3%) for Cmax, 0.416 (38.9%) for AUC(0-last) and 0.421 (38.5%) for AUC(0-inf).

Following a single oral administration of AMX0035 in the fed state concentrations of metabolite PAA were evident from 0.25 h to 0.50 h in all 14 subjects. Maximum plasma concentrations occurred between 2.00 and 4.50 h post-dose. Concentrations then declined in a monophasic/biphasic manner and remained quantifiable until between 6.00 and 8.00 h post-dose. Terminal slopes were reliably determined for all 14 subjects and resultant elimination half-lives ranged between 0.60 and 1.04 h. The geometric mean (geometric CV %) half-life was 0.780 h (16.3%).

The geometric mean (geometric CV %) PAA/PB metabolite to parent ratios following administration in the fed state were 0.407 (45.6%), 0.626 (49.2%) and 0.598 (46.8%) for Cmax, AUC(0-last) and AUC(0-inf), respectively.

### Pharmacokinetics of TURSO, UDCA and GUDCA

TURSO

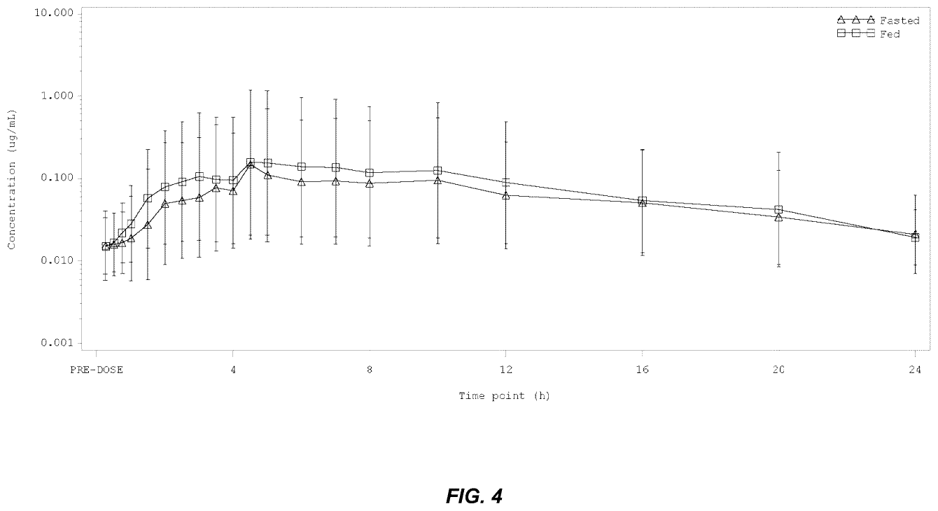

The mean plasma concentration vs time profiles for TURSO are illustrated by food condition on a log₁₀/linear scale for all subjects in the PK analysis set in FIG. 4.

Key PK parameters for TURSO for all subjects in the PK analysis set are presented below in Table 11.

TABLE 11
Summary of Plasma Pharmacokinetic Parameters for TURSO Following Single Oral Doses of AMX0035 to Healthy Male and
Female Subjects in the Fasted and Fed States: Pharmacokinetic Analysis Set

| | | Tmax | Cmax | AUC(0-last) | AUC(0-inf) | AUCextrap | T1/2 | Lambda-z |
|---|---|---|---|---|---|---|---|---|
| Treatment | Statistic | (h) | (ug/mL) | (ug · h/mL) | (ug · h/mL) | (%) | (h) | (1/h) |
| AMX0035 | n | 13 | 13 | 13 | 5 | 5 | 5 | 5 |
| FASTED | Median | 4.500 | 0.936 | 4.94 | 5.15 | 8.554 | 4.408 | 0.1572 |
| (N = 13) | Min | 1.50 | 0.219 | 1.18 | 1.80 | 5.16 | 2.51 | 0.0796 |
| | Max | 10.00 | 1.74 | 11.6 | 6.67 | 18.10 | 8.71 | 0.2765 |
| | Geometric n | NC | 13 | 13 | 5 | 5 | 5 | 5 |
| | Geometric Mean | NC | 0.741 | 4.36 | 3.91 | 8.242 | 4.337 | 0.1598 |
| | Geometric CV % | NC | 71.6 | 79.4 | 58.9 | 53.9 | 49.7 | 49.7 |
| AMX0035 | n | 14 | 14 | 14 | 6 | 6 | 6 | 6 |
| FED | Median | 5.000 | 0.720 | 5.51 | 5.36 | 3.002 | 3.583 | 0.1934 |
| (N = 14) | Min | 4.50 | 0.435 | 2.45 | 3.09 | 1.81 | 1.53 | 0.1293 |
| | Max | 10.00 | 2.16 | 22.4 | 5.83 | 6.12 | 5.36 | 0.4524 |
| | Geometric n | NC | 14 | 14 | 6 | 6 | 6 | 6 |
| | Geometric Mean | NC | 0.762 | 6.13 | 4.84 | 3.149 | 3.359 | 0.2063 |
| | Geometric CV % | NC | 44.0 | 62.7 | 24.1 | 44.2 | 45.1 | 45.1 |

| | | CL/F | Vz/F | Frel Cmax | Frel AUC(0-last) | Frel AUC(0-inf) |
|---|---|---|---|---|---|---|
| Treatment | Statistic | (mL/min) | (L) | (%) | (%) | (%) |
| AMX0035 | n | 5 | 5 | NC | NC | NC |
| FASTED | Median | 3240 | 1710 | NC | NC | NC |
| (N = 13) | Min | 2500 | 1240 | NC | NC | NC |
| | Max | 9260 | 2010 | NC | NC | NC |
| | Geometric n | 5 | 5 | NC | NC | NC |
| | Geometric Mean | 4260 | 1600 | NC | NC | NC |
| | Geometric CV % | 58.9 | 22.1 | NC | NC | NC |
| AMX0035 | n | 6 | 6 | 13 | 13 | 3 |
| FED | Median | 3110 | 1070 | 99.160 | 132.61 | 163.06 |
| (N = 14) | Min | 2860 | 492 | 46.47 | 43.45 | 113.24 |
| | Max | 5400 | 1440 | 224.71 | 334.57 | 171.41 |
| | Geometric n | 6 | 6 | 13 | 13 | 3 |
| | Geometric Mean | 3440 | 1000 | 102.06 | 139.26 | 146.82 |
| | Geometric CV % | 24.1 | 41.2 | 53.0 | 56.9 | 22.9 |

Each treatment comprised one sachet of AMX0035, which contains 1 g TURSO and 3 g sodium PB. NC = Not Calculated.

Following a single oral administration of AMX0035 to healthy male and female subjects in the fasted state, the time matched baseline corrected maximum plasma concentrations for TURSO occurred between 1.50 and 10.00 h post-dose. Terminal slopes were reliably determined for 5 subjects and resultant elimination half-lives ranged between 2.51 and 8.71 h. The geometric mean (geometric CV %) half-life was 4.337 h (49.7%). Where terminal slopes could not be reliably determined, this was a result of an unacceptable coefficient of determination (i.e. adjusted R²<0.9).

The geometric mean (geometric CV %) apparent volume of distribution and apparent plasma clearance were 1600 L (22.1%) and 4260 mL/min (58.9%), respectively.

Following a single oral administration of AMX0035 in the fed state, the time matched baseline corrected maximum plasma concentrations for TURSO occurred between 4.50 and 10.00 h post-dose. Terminal slopes were reliably determined for 6 subjects and resultant elimination half-lives ranged between 1.53 and 5.36 h, with a geometric mean (geometric CV %) of 3.359 h (45.1%). Where terminal slopes could not be reliably determined, this was a result of an unacceptable coefficient of determination (i.e. adjusted R²<0.9).

The geometric mean (geometric CV %) apparent volume of distribution and apparent plasma clearance were 1000 L (41.2%) and 3440 mL/min (24.1%), respectively.

UDCA

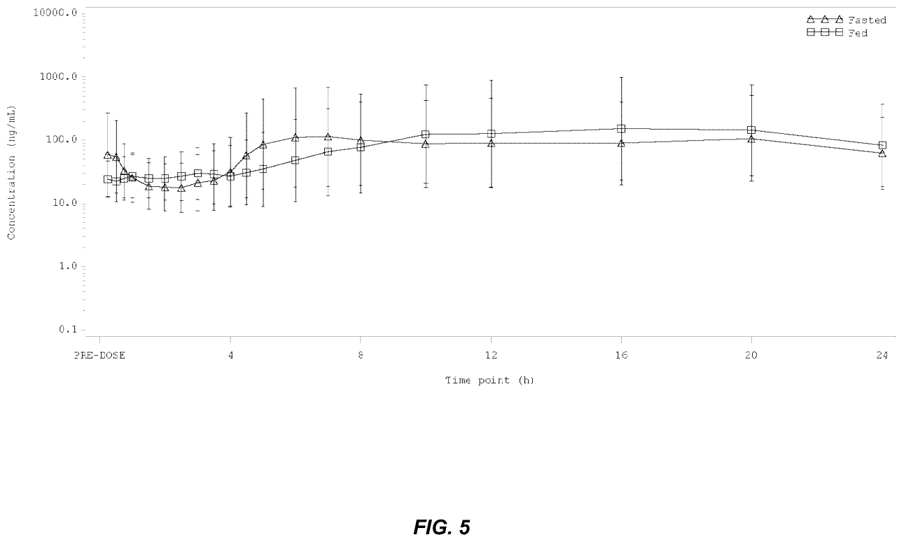

The mean plasma concentration vs time profiles for UDCA are illustrated by food condition on a log₁₀/linear scale for all subjects in the PK analysis set in FIG. 5.

Key PK parameters for UDCA for all subjects in the PK analysis set are presented below in Table 12.

TABLE 12
Summary of Plasma Pharmacokinetic Parameters for UDCA Following Single Oral Doses of AMX0035 to Healthy Male and
Female Subjects in the Fasted and Fed States: Pharmacokinetic Analysis Set

| | | Tmax | Cmax | AUC(0-last) | AUC(0-inf) | AUCextrap | T1/2 | Lambda-z |
|---|---|---|---|---|---|---|---|---|
| Treatment | Statistic | (h) | (ng/mL) | (ng · h/mL) | (ng · h/mL) | (%) | (h) | (1/h) |
| AMX0035 | n | 13 | 13 | 13 | 1 | 1 | 2 | 2 |
| FASTED | Median | 6.000 | 493 | 5370 | 11300 | 2.835 | 5.292 | 0.1610 |
| (N = 13) | Min | 0.25 | 195 | 1970 | 11300 | 2.84 | 3.01 | 0.0915 |
| | Max | 20.00 | 1380 | 14900 | 11300 | 2.84 | 7.58 | 0.2305 |
| | Geometric n | NC | 13 | 13 | 1 | 1 | 2 | 2 |
| | Geometric Mean | NC | 639 | 5540 | 11300 | 2.835 | 4.773 | 0.1452 |
| | Geometric CV % | NC | 73.0 | 72.5 | NC | NC | 73.0 | 73.0 |
| AMX0035 | n | 14 | 14 | 14 | 1 | 1 | 1 | 1 |
| FED | Median | 16.000 | 613 | 7310 | 8580 | 17.944 | 5.312 | 0.1304 |
| (N = 14) | Min | 6.00 | 181 | 1590 | 8580 | 17.94 | 5.31 | 0.1305 |
| | Max | 24.00 | 6250 | 33400 | 8580 | 17.94 | 5.31 | 0.1305 |
| | Geometric n | NC | 14 | 14 | 1 | 1 | 1 | 1 |
| | Geometric Mean | NC | 654 | 6770 | 8580 | 17.944 | 5.312 | 0.1304 |
| | Geometric CV % | NC | 135.2 | 108.3 | NC | NC | NC | NC |

| | | Frel Cmax | Frel AUC(0-last) | Frel AUC(0-inf) | | MR | MR |
|---|---|---|---|---|---|---|---|
| Treatment | Statistic | (%) | (%) | (%) | MR Cmax | AUC(0-last) | AUC(0-inf) |
| AMX0035 | n | NC | NC | NC | 13 | 13 | NC |
| FASTED | Median | NC | NC | NC | 1.274 | 2.023 | NC |
| (N = 13) | Min | NC | NC | NC | 0.14 | 0.22 | NC |
| | Max | NC | NC | NC | 7.50 | 9.60 | NC |
| | Geometric n | NC | NC | NC | 13 | 13 | NC |
| | Geometric Mean | NC | NC | NC | 1.099 | 1.620 | NC |
| | Geometric CV % | NC | NC | NC | 119.9 | 149.3 | NC |
| AMX0035 | n | 13 | 13 | 1 | 14 | 14 | NC |
| FED | Median | 108.36 | 143.12 | 75.818 | 1.060 | 2.064 | NC |
| (N = 14) | Min | 22.77 | 47.58 | 75.82 | 0.18 | 0.22 | NC |
| | Max | 480.00 | 509.25 | 75.82 | 11.67 | 7.00 | NC |
| | Geometric n | 13 | 13 | 1 | 14 | 14 | NC |
| | Geometric Mean | 111.43 | 136.58 | 75.818 | 1.093 | 1.406 | NC |
| | Geometric CV % | 136.6 | 99.2 | NC | 169.5 | 148.8 | NC |

Each treatment comprised one sachet of AMX0035, which contains 1 g TURSO and 3 g sodium PB. NC = Not Calculated

Following a single oral administration of AMX0035 to healthy male and female subjects in the fasted state, the time matched baseline corrected maximum plasma concentrations of metabolite UDCA occurred between 0.25 and 20.00 h post-dose. Terminal slopes were reliably determined for 2 subjects and the resultant elimination half-lives ranged between 3.01 and 7.58 h. Where terminal slopes could not be reliably determined, this was a result of an unacceptable coefficient of determination (i.e. adjusted R²<0.9) or an insufficient number of data points post-Cmax. The geometric mean (geometric CV %) UDCA/TURSO metabolite to parent ratios following administration in the fasted state were 1.099 (119.9%) for Cmax and 1.620 (149.3%) for AUC(0-last).

Following a single oral administration of AMX0035 in the fed state, time matched baseline corrected maximum plasma concentrations of metabolite UDCA occurred between 6.00 and 24.00 h post-dose. Terminal slopes were reliably determined for 1 subject, with a resultant elimination half-life of 5.312 h. Where terminal slopes could not be determined, this was a result of an unacceptable coefficient of determination (i.e. adjusted R²<0.9) or an insufficient number of data points post-Cmax. The geometric mean (geometric CV %) UDCA/TURSO metabolite to parent ratios were 1.093 (169.5%) and 1.406 (148.8%) for Cmax and AUC(0-last), respectively.

GUDCA

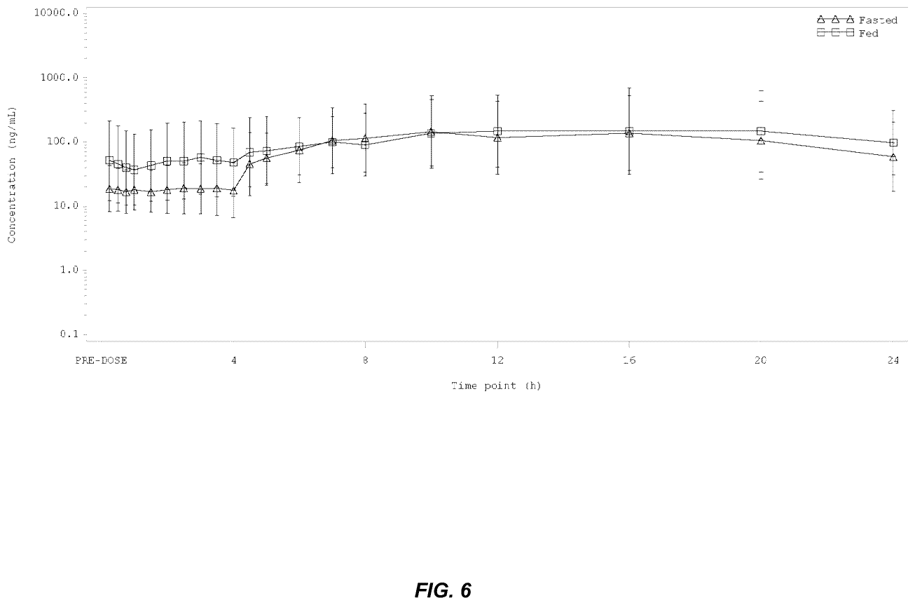

The mean plasma concentration vs time profiles for GUDCA are illustrated by food condition on a log₁₀/linear scale for all subjects in the PK analysis set in FIG. 6.

Key PK parameters for GUDCA for all subjects in the PK analysis set are presented below in Table 13.

TABLE 13
Summary of Plasma Pharmacokinetic Parameters for GUDCA Following Single Oral Doses of AMX0035 to Healthy Male and
Female Subjects in the Fasted and Fed States: Pharmacokinetic Analysis Set

| | | Tmax | Cmax | AUC(0-last) | AUC(0-inf) | AUCextrap | T1/2 | Lambda-z |
|---|---|---|---|---|---|---|---|---|
| Treatment | Statistic | (h) | (ng/mL) | (ng · h/mL) | (ng · h/mL) | (%) | (h) | (1/h) |
| AMX0035 | n | 13 | 13 | 13 | NC | NC | 1 | 1 |
| FASTED | Median | 16.000 | 353 | 3400 | NC | NC | 12.744 | 0.0543 |
| (N = 13) | Min | 6.00 | 143 | 1840 | NC | NC | 12.74 | 0.0544 |
| | Max | 20.00 | 1420 | 12000 | NC | NC | 12.74 | 0.0544 |
| | Geometric n | NC | 13 | 13 | NC | NC | 1 | 1 |
| | Geometric Mean | NC | 381 | 4140 | NC | NC | 12.744 | 0.0543 |
| | Geometric CV % | NC | 76.6 | 70.4 | NC | NC | NC | NC |
| AMX0035 | n | 14 | 14 | 14 | NC | NC | 2 | 2 |
| FED | Median | 16.000 | 505 | 5260 | NC | NC | 17.874 | 0.0454 |
| (N = 14) | Min | 0.50 | 114 | 900 | NC | NC | 11.03 | 0.0280 |
| | Max | 24.00 | 2430 | 18400 | NC | NC | 24.71 | 0.0628 |
| | Geometric n | NC | 14 | 14 | NC | NC | 2 | 2 |
| | Geometric Mean | NC | 463 | 5060 | NC | NC | 16.513 | 0.0419 |
| | Geometric CV % | NC | 91.2 | 88.2 | NC | NC | 62.0 | 62.0 |

| | | Frel Cmax | Frel AUC(0-last) | Frel AUC(0-inf) | | MR | MR |
|---|---|---|---|---|---|---|---|
| Treatment | Statistic | (%) | (%) | (%) | MR Cmax | AUC(0-last) | AUC(0-inf) |
| AMX0035 | n | NC | NC | NC | 13 | 13 | NC |
| FASTED | Median | NC | NC | NC | 0.569 | 1.421 | NC |
| (N = 13) | Min | NC | NC | NC | 0.09 | 0.18 | NC |
| | Max | NC | NC | NC | 2.09 | 2.99 | NC |
| | Geometric n | NC | NC | NC | 13 | 13 | NC |
| | Geometric Mean | NC | NC | NC | 0.571 | 1.056 | NC |
| | Geometric CV % | NC | NC | NC | 107.4 | 114.8 | NC |
| AMX0035 | n | 13 | 13 | NC | 14 | 14 | NC |
| FED | Median | 143.22 | 146.98 | NC | 0.765 | 1.111 | NC |
| (N = 14) | Min | 49.79 | 54.79 | NC | 0.15 | 0.14 | NC |
| | Max | 354.42 | 372.28 | NC | 2.29 | 2.25 | NC |
| | Geometric n | 13 | 13 | NC | 14 | 14 | NC |
| | Geometric Mean | 135.16 | 139.72 | NC | 0.675 | 0.918 | NC |
| | Geometric CV % | 72.6 | 60.0 | NC | 92.9 | 103.4 | NC |

Each treatment comprised one sachet of AMX0035, which contains 1 g TURSO and 3 g sodium PB. NC = Not Calculated.

Following a single oral administration of AMX0035 to healthy male and female subjects in the fasted state, the time matched baseline corrected maximum plasma concentrations of metabolite GUDCA occurred between 6.00 and 20.00 h post-dose. Terminal slopes were reliably determined for 1 subject with a resultant elimination half-life of 12.744 h. Where terminal slopes could not be reliably determined, this was a result of an unacceptable coefficient of determination (i.e. adjusted R²<0.9) or an insufficient number of data points post-Cmax. The geometric mean (geometric CV %) GUDCA/TURSO metabolite to parent ratios following administration in the fasted state were 0.571 (107.4%) for Cmax and 1.056 (114.8%) for AUC(0-last).

Following a single oral administration of AMX0035 in the fed state, the time matched baseline corrected maximum plasma concentrations of metabolite GUDCA occurred between 0.50 and 24.00 h post-dose. Terminal slopes were reliably determined for 2 subjects and the resultant elimination half-lives were 11.03 and 24.71 h. Where terminal slopes could not be determined, this was a result of an unacceptable coefficient of determination (i.e. adjusted R²<0.9) or an insufficient number of data points post-Cmax. The geometric mean (geometric CV %) GUDCA/TURSO metabolite to parent ratios were 0.675 (92.9%) and 0.918 (103.4%) for Cmax and AUC(0-last), respectively.

### Statistical Results and Analysis

PB

The results of the statistical analysis of Cmax, AUC(0-last) and AUC(0-inf) for the assessment of food effect for PB are presented in Table 14.

TABLE 14
Statistical Analysis Results for the Assessment of Food Effect for PB Following Single Oral Doses of
AMX0035 to Healthy Male and Female Subjects in the Fasted and Fed States: Pharmacokinetic Analysis Subset

| | | Fed | | Fasted | | | | | |
|---|---|---|---|---|---|---|---|---|---|
| | | (N = 13) | | (N = 13) | | | | | |
| | | | Adj Geo | | Adj Geo | | | | |
| | | | Mean | | Mean | Ratio (%) | | P-value | CVw (%) |
| Parameter | Comparison | n | (1) | n | (1) | (2) | 90% CI (3) | (4) | (5) |
| Cmax (ug/mL) | Fed/Fasted | 13 | 46.5 | 13 | 191 | 24.35 | (22.25, 26.64) | <0.001 | 12.80 |
| AUC(0-last) | Fed/Fasted | 13 | 109 | 13 | 241 | 45.35 | (41.17, 49.94) | <0.001 | 13.73 |
| (ug · h/mL) | | | | | | | | | |
| AUC(0-inf) | Fed/Fasted | | 120 | 12 | 261 | 45.98 | (41.70, 50.71) | <0.001 | 13.28 |
| (ug · h/mL) | | | | | | | | | |

(1) Adj geo mean = adjusted geometric mean from model,
(2) Ratio of adj geo means for Fed/Fasted,
(3) CI = confidence interval for ratio of adj geo means,
(4) P-value from two-sided test with null hypothesis that ratio is equal to 100%,
(5) CVw = Intra-subject variability.

One subject did not have a reliable estimate of AUC(0-inf) for both fed and fasted treatments (owing to an unacceptable coefficient of determination [i.e. adjusted R²<0.9] for the fed state) and therefore was excluded from the statistical analysis of AUC(0-inf).

The treatment by gender interaction term was not significant at the 5% level for each of Cmax (p=0.52), AUC(0-last) (p=0.21) and AUC(0-inf) (p=0.21) and therefore, this term was dropped from the respective models.

For the comparison of fed vs fasted treatments, the point estimates of the GMRs relating to peak (Cmax) and overall (AUC(0-last) and AUC(0-inf)) exposure levels varied from 24.35% to 45.98%, i.e. levels of exposure for the fed treatment were on average between approximately 24% and 46% of those seen for the fasted treatment. Each of the upper bounds associated with the GMRs were less than 51% and therefore, average levels of exposure for the fed treatment greater than 51% of those seen for the fasted treatment can be ruled out with some confidence.

The differences between the fed and fasted treatments associated with each of the PK parameters were statistically significant at the 10% level, with p-value <0.001 for each of Cmax, AUC(0-last) and AUC(0-inf). As a result, there is statistical evidence to reject the null hypothesis of no difference between fed and fasted treatments.

Whilst no statistically significant gender effects were noted (i.e. p=0.17, p=0.11 and p=0.14 for Cmax, AUC(0-last) and AUC(0-inf), respectively), there was some evidence of higher PB exposure levels in females when compared to males and this was consistent for both fed and fasted regimens (data not shown).

The median of Tmax for the fasted treatment was 0.500 h and for the fed treatment was 1.000 h. The Hodges-Lehmann estimate of the difference between fed and fasted medians (i.e. fed−fasted) was 0.500 h with a 90% CI (0.125, 0.750). The slight increase in Tmax for the fed state when compared to the fasted state was statistically significant (p=0.007) at the 10% level (Table 15).

TABLE 15
Plasma Pharmacokinetic Parameters Using Actual Concentrations: PB
Statistical Analysis Results-Non Parametric Analysis of Tmax
PK Analysis Subset

| | Fasted | | Fed | | Difference | 90% CI | P-value |
|---|---|---|---|---|---|---|---|
| PK Parameter | n | Median | n | Median | (1) | (2) | (3) |
| Tmax (h) | 13 | 0.500 | 13 | 1.000 | 0.500 | (0.125, | 0.007 |
| | | | | | | 0.750) | |

Note:
Each treatment comprised of one sachet of AMX0035 which contains 1 g TURSO (taurursodiol) and 3 g PB (sodium phenylbutyrate)
Results are obtained from a non-parametric Wilcoxon Signed-Rank Test.
(1) Hodges-Lehmann median of Fed-Fasted differences,
(2) Confidence interval for Hodges-Lehmann median of differences,
(3) P-value for the null hypothesis that the difference in Hodges-Lehmann medians is zero.
Hodges-Lehmann median difference is not necessarily the same as the difference between medians.

PAA

The results of the statistical analysis of Cmax, AUC(0-last) and AUC(0-inf) for the assessment of food effect for PAA are presented in Table 16.

TABLE 16
Statistical Analysis Results for the Assessment of Food Effect for PAA Following Single Oral Doses of
AMX0035 to Healthy Male and Female Subjects in the Fasted and Fed States: Pharmacokinetic Analysis Subset

| | | Fed | | Fasted | | | | | |
|---|---|---|---|---|---|---|---|---|---|
| | | (N = 13) | | (N = 13) | | | | | |
| | | | Adj Geo | | Adj Geo | | | | |
| | | | Mean | | Mean | Ratio (%) | | P-value | CVw (%) |
| Parameter | Comparison | n | (1) | n | (1) | (2) | 90% CI (3) | (4) | (5) |
| Cmax (ug/mL) | Fed/Fasted | 13 | 16.1 | 13 | 26.6 | 60.34 | (54.31, 67.05) | <0.001 | 15.01 |
| AUC(0-last) | Fed/Fasted | 13 | 58.2 | 13 | 83.2 | 69.97 | (64.62, 75.75) | <0.001 | 11.28 |
| (ug · h/mL) | | | | | | | | | |
| AUC(0-inf) | Fed/Fasted | 13 | 59.8 | 13 | 84.8 | 70.54 | (65.28, 76.24) | <0.001 | 11.02 |
| (ug · h/mL) | | | | | | | | | |

(1) Adj geo mean = adjusted geometric mean from model,
(2) Ratio of adj geo means for Fed/Fasted,
(3) CI = confidence interval for ratio of adj geo means,
(4) P-value from two-sided test with null hypothesis that ratio is equal to 100%,
(5) CVw = Intra-subject variability.

The treatment by gender interaction term was not significant at the 5% level for each of Cmax (p=0.46), AUC(0-last) (p=0.77) and AUC(0-inf) (p=0.77) and therefore, this term was dropped from the respective models.

For the comparison of fed vs fasted treatments, the point estimates of the GMRs relating to peak (Cmax) and overall (AUC(0-last) and AUC(0-inf)) exposure levels varied from 60.34% to 70.54%. i.e. levels of exposure for the fed treatment were on average between approximately 60% and 71% of those seen for the fasted treatment. Each of the upper bounds associated with the GMRs were less than 77% and therefore, average levels of exposure for the fed treatment greater than 77% of those seen for the fasted treatment can be ruled out with some confidence.

The differences between the fed and fasted treatments associated with each of the PK parameters were statistically significant at the 10% level, with p-value <0.001 for each of Cmax, AUC(0-last) and AUC(0-inf). As a result, there is statistical evidence to reject the null hypothesis of no difference between fed and fasted treatments.

Whilst no statistically significant gender effects were noted (i.e. p=0.24, p=0.18 and p=0.18 for Cmax, AUC(0-last) and AUC(0-inf), respectively), there was some evidence of higher PAA exposure levels in females when compared to males and this was consistent for both fed and fasted regimens (data not shown).

The median of Tmax for both the fasted and fed treatments was 2.500 h. The Hodges-Lehmann estimate of the difference in median values between fed and fasted treatments was 0.250 h with a 90% CI (0.000, 0.750). The slight increase in Tmax for the fed state when compared to the fasted state was statistically significant (p=0.058) at the 10% level (Table 17).

TABLE 17
Plasma Pharmacokinetic Parameters Using Actual Concentrations: PAA
Statistical Analysis Results-Non Parametric Analysis of Tmax
PK Analysis Subset

| PK | Fasted | | Fed | | Difference | 90% CI | P-value |
|---|---|---|---|---|---|---|---|
| Parameter | n | Median | n | Median | (1) | (2) | (3) |
| Tmax (h) | 13 | 2.500 | 13 | 2.500 | 0.250 | (0.000, | 0.058 |
| | | | | | | 0.750) | |

Note:
Each treatment comprised of one sachet of AMX0035 which contains 1 g TURSO (taurursodiol) and 3 g PB (sodium phenylbutyrate)
Results are obtained from a non-parametric Wilcoxon Signed-Rank Test.
(1) Hodges-Lehmann median of Fed-Fasted differences,
(2) Confidence interval for Hodges-Lehmann median of differences,
(3) P-value for the null hypothesis that the difference in Hodges-Lehmann medians is zero.
Hodges-Lehmann median difference is not necessarily the same as the difference between medians.

TURSO

The results of the statistical analysis of Cmax and AUC(0-last) based on time matched baseline corrected concentrations for the assessment of food effect for TURSO are presented in Table 18.

TABLE 18
Statistical Analysis Results for the Assessment of Food Effect for TURSO Following Single Oral Doses of
AMX0035 to Healthy Male and Female Subjects in the Fasted and Fed States: Pharmacokinetic Analysis Subset

| | | Fed | | Fasted | | | | | |
|---|---|---|---|---|---|---|---|---|---|
| | | (N = 13) | | (N = 13) | | | | | |
| | | | Adj Geo | | Adj Geo | | | | |
| | | | Mean | | Mean | Ratio (%) | | P-value | CVw (%) |
| Parameter | Comparison | n | (1) | n | (1) | (2) | 90% CI (3) | (4) | (5) |
| Cmax (ug/mL) | Fed/Fasted | 13 | 0.767 | 13 | 0.755 | 101.56 | (78.52, 131.36) | 0.92 | 37.66 |
| AUC(0-last) | Fed/Fasted | 13 | 6.19 | 3 | 4.46 | 138.81 | (105.39, 182.83) | 0.056 | 40.52 |
| (ug · h/mL) | | | | | | | | | |

(1) Adj geo mean = adjusted geometric mean from model,
(2) Ratio of adj geo means for Fed/Fasted,
(3) CI = confidence interval for ratio of adj geo means,
(4) P-value from two-sided test with null hypothesis that ratio is equal to 100%,
(5) CVw = Intra-subject variability.

The number of subjects with reliable estimates of AUC(0-inf) for both periods was less than 7: therefore, no formal statistical analysis of AUC(0-inf) was performed.

The treatment by gender interaction term was not significant at the 5% level for each of Cmax (p=0.74) and AUC(0-last) (p=0.96) and therefore, this term was dropped from the respective models.

The GMRs (90% CIs) for Fed/Fasted were 101.56% (78.52%, 131.36%) and 138.81% (105.39%, 182.83%) for Cmax and AUC(0-last), respectively, i.e. average levels of exposure as measured by Cmax and AUC(0-last) for the fed treatment were on average approximately 2% and 39% higher, respectively, than those seen for the fasted treatment. However, these estimates should be viewed with some caution due to the width of the associated 90% CIs, e.g. average increases of AUC(0-last) as small as 5% and as large as 83% cannot be ruled out with confidence.

The difference between the fed and fasted treatments associated with Cmax was not statistically significant at the 10% level, i.e. p=0.92. For the analysis of overall exposure across the entire sampling period, i.e. AUC(0-last), the difference between fed and fasted treatments was statistically significant at the 10% level (p=0.056) i.e. there was no statistical evidence to reject the null hypothesis of no food effect for Cmax and evidence to reject the null hypothesis of no food effect for AUC(0-last).

There was some evidence of higher TURSO exposure levels in females when compared to males and this was consistent for both fed and fasted regimens, i.e. this effect was significant for AUC(0-last) at the 10% level (p=0.060) but was not statistically significant for Cmax (p=0.13) (data not shown).

The median of Tmax based on time matched baseline corrected concentrations for the fasted treatment was 4.500 h and for the fed treatment was 5.000 h. The Hodges-Lehmann estimate of the difference between fed and fasted medians was 2.000 h with a 90% CI (0.250, 2.758). The increase in Tmax for the fed state when compared to the fasted state was statistically significant (p=0.052) at the 10% level (Table 19).

TABLE 19
Plasma Pharmacokinetic Parameters Using Time Matched Baseline Corrected Concentrations: TURSO
Statistical Analysis Results-Non Parametric Analysis of Tmax
PK Analysis Subset

| | Fasted | | Fed | | Difference | | P-value |
|---|---|---|---|---|---|---|---|
| PK Parameter | n | Median | n | Median | (1) | 90% CI (2) | (3) |
| Tmax (h) | 13 | 4.500 | 13 | 5.000 | 2.000 | (0.250, 2.758) | 0.052 |

Note:
Each treatment comprised of one sachet of AMX0035 which contains 1 g TURSO (taurursodiol) and 3 g PB (sodium phenylbutyrate)
Results are obtained from a non-parametric Wilcoxon Signed-Rank Test.
(1) Hodges-Lehmann median of Fed-Fasted differences,
(2) Confidence interval for Hodges-Lehmann median of differences,
(3) P-value for the null hypothesis that the difference in Hodges-Lehmann medians is zero.
Hodges-Lehmann median difference is not necessarily the same as the difference between medians.

UDCA

The results of the statistical analysis of Cmax and AUC(0-last) based on time matched baseline corrected concentrations for the assessment of food effect for UDCA are presented in Table 20.

TABLE 20
Statistical Analysis Results for the Assessment of Food Effect for UDCA Following Single Oral Doses of
AMX0035 to Healthy Male and Female Subjects in the Fasted and Fed States: Pharmacokinetic Analysis Subset

| | | Fed | | Fasted | | | | | |
|---|---|---|---|---|---|---|---|---|---|
| | | (N = 13) | | (N = 13) | | | | | |
| | | | Adj Geo | | Adj Geo | | | | |
| | | | Mean | | Mean | Ratio (%) | | P-value | CVw (%) |
| Parameter | Comparison | n | (1) | n | (1) | (2) | 90% CI (3) | (4) | (5) |
| Cmax (ng/mL) | Fed/Fasted | 13 | 712 | 13 | 635 | 112.17 | (65.80, 191.21) | 0.71 | 87.65 |
| AUC(0-last) | Fed/Fasted | 13 | 7540 | 13 | 5530 | 136.34 | (88.53, 209.98) | 0.22 | 67.30 |
| (ng · h/mL) | | | | | | | | | |

(1) Adj geo mean = adjusted geometric mean from model,
(2) Ratio of adj geo means for Fed/Fasted,
(3) CI = confidence interval for ratio of adj geo means,
(4) P-value from two-sided test with null hypothesis that ratio is equal to 100%,
(5) CVw = Intra-subject variability.

The number of subjects with reliable estimates of AUC(0-inf) for both periods was less than 7: therefore, no formal statistical analysis of AUC(0-inf) was performed.

The treatment by gender interaction term was not significant at the 5% level for each of Cmax (p=0.45) and AUC(0-last) (p=0.86) and therefore, this term was dropped from the respective models.

The GMRs (90% CIs) for Fed/Fasted were 112.17% (65.80%, 191.21%) and 136.34% (88.53%, 209.98%) for Cmax and AUC(0-last), respectively, i.e. average levels of exposure as measured by Cmax and AUC(0-last) for the fed treatment were on average approximately 12% and 36% higher, respectively, than those seen for the fasted treatment. However, these estimates should be viewed with some caution due to the width of the associated 90% CIs, e.g. average decreases in AUC(0-last) as small as 11% and average increases in AUC(0-last) as large as 110% (i.e. more than double) cannot be ruled out with confidence.

The differences between the fed and fasted treatments associated with each of the PK parameters were not statistically significant at the 10% level, i.e. p=0.71 for Cmax and 0.22 for AUC(0-last). As a result, there is no statistical evidence of a difference between fed and fasted treatments i.e. no evidence to reject the null hypothesis of no food effect for each of Cmax and AUC(0-last).

Whilst no statistically significant gender effects were noted (i.e. p=0.13 and p=0.16 for Cmax and AUC(0-last), respectively), there was some evidence of higher UDCA exposure levels in males when compared to females and this was consistent for both fed and fasted regimens (data not shown).

The median of Tmax based on time matched baseline corrected concentrations for the fasted treatment was 6.000 h and for the fed treatment was 16.000 h. The Hodges-Lehmann estimate of the difference between fed and fasted medians was 7.000 h with a 90% CI (2.000, 11.500). The increase in Tmax for the fed state when compared to the fasted state was statistically significant (p=0.022) at the 10% level (Table 21).

TABLE 21
Plasma Pharmacokinetic Parameters Using Time Matched Baseline Corrected Concentrations: UDCA
Statistical Analysis Results-Non Parametric Analysis of Tmax
PK Analysis Subset

| | Fasted | | Fed | | Difference | | P-value |
|---|---|---|---|---|---|---|---|
| PK Parameter | n | Median | n | Median | (1) | 90% CI (2) | (3) |
| Tmax (h) | 13 | 6.000 | 13 | 16.000 | 7.000 | (2.000, 11.500) | 0.022 |

Note:
Each treatment comprised of one sachet of AMX0035 which contains 1 g TURSO (taurursodiol) and 3 g PB (sodium phenylbutyrate)
Results are obtained from a non-parametric Wilcoxon Signed-Rank Test.
(1) Hodges-Lehmann median of Fed-Fasted differences,
(2) Confidence interval for Hodges-Lehmann median of differences,
(3) P-value for the null hypothesis that the difference in Hodges-Lehmann medians is zero.
Hodges-Lehmann median difference is not necessarily the same as the difference between medians.

GUDCA

The results of the statistical analysis of Cmax and AUC(0-last) based on time matched baseline corrected concentrations for the assessment of food effect for GUDCA are presented in Table 22.

TABLE 22
Statistical Analysis Results for the Assessment of Food Effect for GUDCA Following Single Oral Doses of
AMX0035 to Healthy Male and Female Subjects in the Fasted and Fed States: Pharmacokinetic Analysis Subset

| | | Fed | | Fasted | | | | | |
|---|---|---|---|---|---|---|---|---|---|
| | | (N = 13) | | (N = 13) | | | | | |
| | | | Adj Geo | | Adj Geo | Ratio (%) | | P-value | CVw (%) |
| Parameter | Comparison | n | Mean (1) | n | Mean (1) | (2) | 90% CI (3) | (4) | (5) |
| Cmax (ng/mL) | Fed/Fasted | 13 | 514 | 13 | 382 | 134.45 | (95.95, 188.41) | 0.14 | 50.62 |
| AUC(0-last) | Fed/ Fasted | 13 | 5760 | 13 | 4200 | 137.31 | (105.64, 178.49) | 0.053 | 38.44 |
| (ng · h/mL) | | | | | | | | | |

(1) Adj geo mean = adjusted geometric mean from model,
(2) Ratio of adj geo means for Fed/Fasted,
(3) CI = confidence interval for ratio of adj geo means,
(4) P-value from two-sided test with null hypothesis that ratio is equal to 100%,
(5) CVw = Intra-subject variability

The number of subjects with reliable estimates of AUC(0-inf) for both periods was less than 7: therefore, no formal statistical analysis of AUC(0-inf) was performed.

The treatment by gender interaction term was not significant at the 5% level for each of Cmax (p=0.77) and AUC(0-last) (p=0.83) and therefore, this term was dropped from the respective models.

The GMRs (90% CIs) for Fed/Fasted were 134.45% (95.95%, 188.41%) and 137.31% (105.64%, 178.49%) for Cmax and AUC(0-last), respectively, i.e. average levels of exposure as measured by Cmax and AUC(0-last) for the fed treatment were on average approximately 34% and 37% higher, respectively, than those seen for the fasted treatment. However, these estimates should be viewed with some caution due to the width of the associated 90% CIs, e.g. average decreases in Cmax as small as 4% and average increases in AUC(0-last) as large as 78% cannot be ruled out with confidence.

The difference between the fed and fasted treatments associated with Cmax was not statistically significant at the 10% level (p=0.14), i.e., no evidence to reject the null hypothesis of no food effect for Cmax. For the analysis of overall exposure across the entire sampling period, i.e. AUC(0-last), the difference between fed and fasted treatments was statistically significant at the 10% level (p=0.053) i.e. evidence to reject the null hypothesis of no food effect for AUC(0-last) (data not shown).

The median of Tmax based on time matched baseline corrected concentrations for both the fasted and fed treatments was 16.000 h. The Hodges-Lehmann estimate of the difference between fed and fasted regimens was 2.000 h with a 90% CI (−4.000, 5.000). This difference was not statistically significant at the 10% level (p=0.66) (Table 23).

TABLE 23
Plasma Pharmacokinetic Parameters Using Time Matched Baseline Corrected Concentrations: GUDCA
Statistical Analysis Results-Non Parametric Analysis of Tmax
PK Analysis Subset

| | Fasted | | Fed | | Difference | 90% CI | P-value |
|---|---|---|---|---|---|---|---|
| PK Parameter | n | Median | n | Median | (1) | (2) | (3) |
| Tmax (h) | 13 | 16.000 | 13 | 16.000 | 2.000 | (−4.000, 5.000) | 0.66 |

Note:
Each treatment comprised of one sachet of AMX0035 which contains 1 g TURSO (taurursodiol) and 3 g PB (sodium phenylbutyrate)
Results are obtained from a non-parametric Wilcoxon Signed-Rank Test.
(1) Hodges-Lehmann median of Fed-Fasted differences,
(2) Confidence interval for Hodges-Lehmann median of differences,
(3) P-value for the null hypothesis that the difference in Hodges-Lehmann medians is zero.
Hodges-Lehmann median difference is not necessarily the same as the difference between medians.

### Statistical/Analytical Issues

The distributional assumptions underlying the statistical analysis appeared to be satisfied for the analysis of the different PK parameters with the possible exception of PAA Cmax, i.e. some evidence that the residuals from the model fit were not normally distributed. Further examination of the residuals indicated no particular reason for this deviation from the underlying assumptions and the results are consistent with other PAA PK parameters.

There were no other statistical analytical issues.

### Pharmacokinetic and Statistical Conclusions

PB and PAA

Following single oral administrations of AMX0035, plasma exposure to PB, based on Cmax, AUC(0-last) and AUC(0-inf), showed statistically significant decreases of approximately 76%, 55% and 54%, respectively, in the fed state relative to the fasted state.

Under fasted conditions, peak exposure to metabolite PAA was approximately 14% of that for PB, while total exposure based on AUC(0-inf), was approximately 35% of that for PB. PAA/PB metabolite to parent ratios were higher in the fed state with peak exposure approximately 34% of that for PB and total exposure, based on AUC(0-inf) approximately 48% of that for PB.

A significant increase in PB and PAA Tmax was noted for the fed state when compared to the fasted state albeit the increases were slight i.e. difference in median values of 0.5 h or less.

Following administration in the fed state, the geometric mean Cmax, AUC(0-last) and AUC(0-inf) for metabolite PAA showed statistically significant decreases of approximately 40%, 30% and 29%, respectively, compared to the fasted state.

TURSO, UDCA and GUDCA

Following single oral administrations of AMX0035, peak plasma exposure to TURSO, based on Cmax was relatively unchanged whilst overall exposure based on AUC(0-last) showed a statistically significant increase of approximately 39% in the fed state relative to the fasted state.

Under fasted conditions, conversion of TURSO to UDCA was extensive, with peak exposure to metabolite UDCA approximately 86% of that for TURSO and total exposure, based on AUC(0-last), was approximately 127% of that for TURSO. This was largely unchanged in the fed state, with peak exposure approximately 86% of that for TURSO and total exposure, based on AUC(0-last), approximately 110% of that for TURSO.

Following administration in the fed state, the geometric mean Cmax and AUC(0-last) for metabolite UDCA showed increases of approximately 12% and 36%, respectively, compared to the fasted state. However, due to the high variability these changes did not achieve statistical significance.

Under fasted conditions, peak exposure to metabolite GUDCA was approximately 51% of that for TURSO and total exposure, based on AUC(0-last), was approximately 95% of that for TURSO. This was largely unchanged in the fed state, with peak exposure approximately 61% of that for TURSO and total exposure, based on AUC(0-last), approximately 83% of that for TURSO.

Following administration in the fed state, the geometric mean Cmax and AUC(0-last) for metabolite GUDCA showed increases of approximately 34% and 37%, respectively, in the fed state; however, only the difference in AUC(0-last) achieved statistical significance.

A significant increase in UDCA Tmax based on time matched baseline corrected concentrations was noted for the fed state when compared to the fasted state. A similar trend, albeit with smaller increases, was seen for TURSO and GUDCA Tmax but only for TURSO did this increase achieve statistical significance.

### Discussion and Overall Conclusions

PB and PAA

Following a single oral administration of AMX0035 at in the fasted state, PB was rapidly absorbed with median Tmax occurring at 0.500 h post-dose for all subjects. The resultant profile was characterized by geometric mean volume of distribution and total clearance of 8.4 L and 211 mL/min, respectively. The terminal elimination was also rapid, with a geometric mean plasma half-life of 0.461 h.

The inter-subject variability associated with exposure (measured by Cmax, AUC(0-last) and AUC(0-inf)) was moderate, with geometric mean CV % ranging from 39.0% to 45.2%.

Administration of AMX0035 with a standardized high fat breakfast resulted in a slight delay in absorption of PB, delaying the median Tmax by 0.500 h compared to the fasted state, to a median of 1.000 h, which is suspected to be as a result of slowed gastric emptying following food. Peak and overall plasma exposure, based on Cmax, AUC(0-last) and AUC(0-inf), showed statistically significant decreases following food of approximately 76%, 55% and 54%, respectively, compared to the fasted state. Inter-subject variability associated with exposure remained consistent between food conditions with Cmax, AUC(0-last) and AUC(0-inf) geometric mean CV % ranging between 28.6% and 45.4%. Terminal elimination of PB also appeared unchanged in the fed state, with geometric mean terminal half-life of 0.599 h.

In contrast, the volume of distribution and total clearance of PB increased to 21.3 L and 410 mL/min, respectively, when dosed in the fed state which, when combined with the reduced bioavailability, could be indicative of linear kinetics. Exposure to PB in both the fed and fasted food conditions showed slight differences between males and females; however, due to the moderate variability between the subjects, which was observed to be higher in males than females, this was not conclusive and statistical analysis indicated no statistically significant difference between males and females.

Following administration of AMX0035 under fasted conditions, peak exposure to metabolite PAA was 0.168-fold of that for PB, while total exposure based on AUC(0-inf), was 0.421-fold of that for PB. The plasma half-life of PAA was comparable to the rapid half-life noted for PB, suggesting elimination of PAA may be formation rate limited.

The time taken to obtain maximum concentrations of PAA was unchanged following administration of AMX0035 in the fed state, with a median of 2.500 h for both food conditions. However, in contrast, the adjusted geometric mean Cmax, AUC(0-last) and AUC(0-inf) showed statistically significant decreases of approximately 40%, 30% and 29%, respectively, compared to the fasted state. The reduction in exposure for PAA was less than that observed for parent PB; this was reflected in the metabolite to parent ratios, which were reported to be slightly higher in the fed state with peak exposure 0.407-fold of that for PB and total exposure, based on AUC(0-inf), being 0.598-fold of that for PB. The estimated geometric mean half-life was almost unchanged by the food condition at 0.780 h. Similar to PB, slight differences were noted between males and females in both food conditions; however, these differences were not conclusive, and statistical analysis indicated there were no statistically significant differences observed.

One subject showed decreased exposure to PB compared to the rest of the group, following administration with AMX0035 in both the fasted and fed states. Maximum plasma PB was 2.92 and 3.63 fold lower than the group geometric mean in the fasted and fed states, respectively. Overall exposure, based on AUC(0-last) and AUC(0-inf) were both 3.2-fold lower than the group geometric mean in the fasted state, and 3.2- and 3.4-fold lower than the group geometric mean in the fed state, respectively. In addition, due to the appearance of the curve for one subject, their AUC(0-inf) values were considered unreliable.

TURSO, UDCA and GUDCA

Following a single oral administration of AMX0035 in the fasted state, TURSO was absorbed with median Tmax occurring at 4.500 h post-dose. The resultant profile was characterized by geometric mean volume of distribution and total clearance of 1600 L and 4260 mL/min, respectively. The geometric mean plasma half-life of TURSO was 4.337 h in the fasted state.

The inter-subject variability associated with exposure (measured by Cmax, AUC(0-last) and AUC(0-inf)) was high, with geometric mean CV % ranging from 58.9% to 79.4%.

Administration of AMX0035 with a standardized high fat breakfast resulted in a slightly delayed absorption of TURSO, as demonstrated by the median Tmax increasing from 4.500 h in the fasted state to 5.000 h in the fed state. This delay is suspected to be a result of slowed gastric emptying following food. In the fed state, peak plasma exposure, based on Cmax was unchanged, however, overall exposure based on AUC(0-inf) and AUC(0-last) showed an increase of approximately 47% and 39%, respectively. However, statistical analysis could only be performed on AUC(0-last) due to limited availability of AUC(0-inf) data. Inter-subject variability associated with exposure was slightly reduced in the fed state, with geometric CV % ranging between 24.1% and 62.7% for Cmax, AUC(0-last) and AUC(0-inf). Terminal elimination of TURSO was unchanged in the fed state, with geometric mean terminal T1/2 of 3.359 h.

The volume of distribution and total clearance of TURSO decreased to 1000 L and 3440 mL/min, respectively, when dosed in the fed state; when combined with the increased bioavailability seen for AUC, this could be indicative of linear kinetics. The inter-subject variability noted following exposure to TURSO showed little difference between males and females in both the fed and fasted states. As a result, statistical analysis indicated there was no statistically significant difference between males and females.

Following administration of AMX0035 under fasted conditions, peak exposure to metabolite UDCA was 1.099-fold of that for TURSO and total exposure, based on AUC(0-last), was 1.620-fold of that for TURSO indicating extensive and complete metabolism of TURSO to UDCA. The plasma half-life of UDCA was comparable to the half-life of TURSO, suggesting elimination of UDCA may be formation rate limited.

The time taken to obtain maximum concentrations of UDCA was delayed following administration of AMX0035 in the fed state, with a median of 16.000 h, relative to 6.000 h in the fasted state. However, in contrast, the geometric mean Cmax and AUC(0-last) showed an increase of approximately 12% and 36%, respectively. The increase in overall exposure was approximately equivalent for UDCA to that observed for parent TURSO. This was reflected in the metabolite to parent ratios, which remained unchanged in the fed state, with peak exposure 1.093-fold of that for TURSO and total exposure, based on AUC(0-last), 1.406-fold of that for TURSO. The estimated geometric mean half-life was also unchanged by the food condition at 5.312 h. The inter-subject variability noted following exposure to UDCA showed little difference between males and females in both the fed and fasted states. As a result, statistical analysis indicated there was no statistically significant difference observed between males and females.

The peak exposure to metabolite GUDCA, following administration with AMX0035 under fasted conditions, was 0.571-fold of that for TURSO and total exposure, based on AUC(0-last), was 1.056-fold of that for TURSO, suggesting complete and extensive metabolism of TURSO to UDCA, then to GUDCA. The plasma half-life of GUDCA was extended, relative to the half-life of TURSO (12.744 h vs 4.337 h), suggesting elimination of GUDCA was not formation rate limited.

The time taken to obtain maximum concentrations of GUDCA was unchanged with a median of 16.000 h, following administration of AMX0035 in the fed state. However, in contrast, the geometric mean Cmax and AUC(0-last) showed increases of 34% and 37%, respectively, in the fed state. However, due to the high variability observed, it was considered statistically significant for AUC(0-last) only. The increase in overall exposure for GUDCA was equivalent to that observed for parent TURSO. This was reflected in the metabolite to parent ratios, which were largely unchanged in the fed state with peak exposure 0.675-fold of that for TURSO and total exposure, based on AUC(0-last), 0.918-fold of that for TURSO.

Similar to TURSO and UDCA, the inter-subject variability noted following exposure to GUDCA showed only slight differences between males and females in both food conditions. As a result, statistical analysis indicated there was no statistically significant difference observed between males and females.

Safety Discussion

There were no safety concerns associated with administration of AMX0035 in the fasted or the fed states.

There were no serious or severe AEs or ADRs. Overall, 1 subject experienced a moderate, unrelated AE which resulted in the withdrawal of IMP.

With the exception of 1 (7.1%) subject in the fed treatment group, no subject had an increase in QTcF from baseline >30 msec. No subjects recorded QTcF values that were ≥450 msec at any time point and no subject had an increase in QTcF >60 msec.

There is no evidence to suggest that AMX0035 dosing in either the fasted or fed state is associated with any clinically relevant QTcF effect within the observed range of plasma concentrations, i.e. PB up to approximately 200 μg/mL in the fasted state and PAA up to approximately 40 μg/mL in the fed state.

There were no clinically significant findings in any clinical laboratory assessments, vital signs, ECGs or physical examination findings.

### Overall Conclusions

Pharmacokinetic Conclusions

PB and PAA

Following single oral administrations of AMX0035, plasma exposure to PB, based on Cmax, AUC(0-last) and AUC(0-inf), showed statistically significant decreases of approximately 76%, 55% and 54%, respectively, in the fed state relative to the fasted state.

Under fasted conditions, peak exposure to metabolite PAA was approximately 14% of that for PB, while total exposure based on AUC(0-inf), was approximately 35% of that for PB. PAA/PB metabolite to parent ratios were higher in the fed state with peak exposure approximately 34% of that for PB and total exposure, based on AUC(0-inf) approximately 48% of that for PB.

A significant increase in PB and PAA Tmax was noted for the fed state when compared to the fasted state albeit the increases were slight i.e. difference in median values of 0.5 h or less.

Following administration in the fed state, the geometric mean Cmax, AUC(0-last) and AUC(0-inf) for metabolite PAA showed statistically significant decreases of approximately 40%, 30% and 29%, respectively, compared to the fasted state.

TURSO. UDCA and GUDCA Following single oral administrations of AMX0035, peak plasma exposure to TURSO, based on Cmax was relatively unchanged whilst overall exposure based on AUC(0-last) showed a statistically significant increase of approximately 39% in the fed state relative to the fasted state.

Under fasted conditions, conversion of TURSO to UDCA was extensive, with peak exposure to metabolite UDCA approximately 86% of that for TURSO and total exposure, based on AUC(0-last), was approximately 127% of that for TURSO. This was largely unchanged in the fed state, with peak exposure approximately 86% of that for TURSO and total exposure, based on AUC(0-last), approximately 110% of that for TURSO.

Following administration in the fed state, the geometric mean Cmax and AUC(0-last) for metabolite UDCA showed increases of approximately 12% and 36%, respectively, compared to the fasted state. However, due to the high variability these changes did not achieve statistical significance.

Under fasted conditions, peak exposure to metabolite GUDCA was approximately 51% of that for TURSO and total exposure, based on AUC(0-last), was approximately 95% of that for TURSO. This was largely unchanged in the fed state, with peak exposure approximately 61% of that for TURSO and total exposure, based on AUC(0-last), approximately 83% of that for TURSO.

Following administration in the fed state, the geometric mean Cmax and AUC(0-last) for metabolite GUDCA showed increases of approximately 34% and 37%, respectively, in the fed state; however, only the difference in AUC(0-last) achieved statistical significance.

A significant increase in UDCA Tmax based on time matched baseline corrected concentrations was noted for the fed state when compared to the fasted state. A similar trend, albeit with smaller increases, was seen for TURSO and GUDCA Tmax but only for TURSO did this increase achieve statistical significance.

### Safety Conclusions

AMX0035 administered in the fasted and fed states was well tolerated under the conditions of this study.

One (7.1%) subject reported 1 TEAE following administration of AMX0035 in the fed treatment group. The AE of musculoskeletal pain was moderate (Grade 2) in severity, unrelated to AMX0035 and led to the withdrawal of IMP.

There were no IMP-related AEs reported during this study.

There is no evidence to suggest that AMX0035 dosing in either the fasted or fed state is associated with any clinically relevant QTcF effect within the observed range of plasma concentrations, i.e. PB up to approximately 200 μg/mL in the fasted state and PAA up to approximately 40 μg/mL in the fed state.

No clinically significant changes in clinical laboratory assessments, vital signs, ECGs or physical examination findings were reported.

### Example 3: Population Pharmacokinetics of Phenylbutyrate (PB) and Phenylacetate (PAA) Following the Administration of AMX0035 for the Treatment of ALS

The objectives of the study were to (1) develop a population pharmacokinetic (PK) model to describe the plasma concentrations of PB and its metabolite, PAA, in patients with ALS receiving AMX0035; (2) Identify demographic and clinical characteristics explaining the PK variability of PB and PAA; and (3) Perform simulations with the final population PK model to examine the influence of different covariates on PB and PAA exposure.

Pharmacokinetic data for the population PK analysis were obtained from a Phase 1 food effect study in healthy adult volunteers and a Phase 2 clinical trial in patients with ALS. The phase 1 food effect study examined the PK of PB, TUDCA and major metabolites following single oral dose administration of AMX0035 with and without food in healthy subjects. Subjects received a single-dose of 1 sachet (1 g TUDCA and 3 g PB) of AMX0035 under fasting (overnight and 4 hours post-dose) and fed (standard high-fat breakfast 30 minutes prior to dose) conditions with a minimum 4-day washout between treatments. The order of administration with and without food was randomly assigned. Serial blood samples for analysis of PB, PAA, TUDCA, UDCA, and GUDCA plasma concentrations were collected for 24 hours following each dose. Additionally, plasma samples were obtained on the day prior to the first dose of AMX0035 to characterize endogenous concentrations of TUDCA and metabolites. Referred to as protocol A35-002.

The Phase 2 trial examined the safety, tolerability, efficacy, and biological activity of AMX0035. Subjects were randomly assigned in a 2:1 ratio to receive AMX0035 or placebo. Treatment was administered as 1 sachet of AMX0035 or placebo daily for the initial 3 weeks and then increased, if tolerated, to 1 sachet twice daily. Subjects were advised to take the drug before a meal. A sparse sampling strategy comprised of single plasma samples collected at the baseline visit (predose) and at the 12- and 24-week treatment visits was used for analysis of plasma concentrations for PB, PAA, TUDCA, UDCA, and GUDCA. Referred to as protocol 3500.

### Results

### Summary

A one-compartment model with first-order absorption, first-order metabolism of PB to PAA (100% conversion), a metabolism (transit) compartment to model the delay in appearance of metabolite plasma concentration and non-linear elimination of PAA best described the PB and PAA plasma concentration data. The final model included covariates describing the influence of food administration on oral absorption rate constant (Ka), food administration on apparent metabolic clearance of PB to PAA (CL_{PB}/F) or apparent volume of distribution of PB (V_{PB}/F), body weight on maximum rate of elimination of PAA (Vₘₐₓ−PAA), and a diagnosis of ALS on VₘₐₓPAA and volume of distribution of PAA (V_{PAA}). No inter-occasion variability (IOV) was included.

Parameter estimates for the final model and the bootstrap analysis are presented in Table 24. PB terminal elimination half-life is estimated to be 0.45 h based on typical values for CLPB/F and VPB/F. The final model projected a 52.4% (95% confidence interval: 48.5%-56.3%) decrease in relative bioavailability and 60% (95% confidence interval: 56%-64%) decrease in the Ka when PB is administered under fed versus fasting conditions.

For the Phase 1 study, predicted individual maximum plasma concentration (Cₘₐₓ) and area under the plasma concentration versus time curve from time 0 to infinity (AUC_{0-∞}) values based on post hoc (empirical Bayesian) PK parameter estimates were in close agreement with the corresponding observed individual Cₘₐₓ and AUC_{0-∞} values for both fed and fasted conditions.

This finding along with the lack of accumulation upon multiple dosing support the use of this approach to estimate subject-specific exposure parameters at steady state in ALS patients. Simulations were performed to evaluate the influence of the covariates included in the final model on systemic exposure to PB and PAA. The only covariate impacting PB exposure was drug administration in relation to food. Model-predicted PB Cₘₐₓ and area under the plasma concentration versus time curve from time 0 to the time of the last quantifiable plasma concentration (AUC₀₋ₗₐₛₜ) were 3.2- and 2.2-fold lower, respectively, when administered with food relative to when administered fasting.

Food administration, ALS diagnosis and body weight influenced PAA exposure. Model predicted PAA Cₘₐₓ and AUC₀₋ₗₐₛₜ were 1.5- and 1.3-fold lower, respectively, when administered with food relative to when administered fasting. The absence of an ALS diagnosis had only a small effect (<1.2-fold) on PAA Cₘₐₓ and AUC₀₋ₗₐₛₜ. Model-based simulations indicate that body weight had a marked effect on PAA exposure. PAA Cmax was 1.45-fold higher at 50-kg body weight and 1.47-fold lower at 115 kg compared to 70 kg, and PAA AUC0-last was 1.87-fold higher at 50 kg and 1.72-fold lower at 115 kg compared to 70 kg.

The results demonstrate that administration of AMX0035 with food decreases PB and, to a lesser extent, PAA exposure in plasma relative to administration in a fasted state. PB and PAA PK are generally similar in ALS patients and healthy subjects. PB exposure is not appreciably affected by body weight while there is an inverse relationship between PAA exposure and body weight.

TABLE 24
Population Pharmacokinetic Parameter Estimates From the
Final Model and Bootstrap Analysis of the Final Model

| | Bootstrap | |
|---|---|---|
| | | 2.5^{th}-97.5^{th} |
| Final Model | Median | Percentiles |

Fixed-Effect Parameter *

| | | | | |
|---|---|---|---|---|
| CL [symbol] /F (L/h) | 14.8 | (9.9) | 14.7 | 11.8-18.6 |
| V [symbol] /F (L) | 9.6 | (16.7) | 9.7 | 6.3-14.5 |
| V [symbol] (L) | 56.3 | (6.7) | 56.1 | 48.3-65.9 |
| Ka [symbol] (h [symbol] ) | 1.92 | (8.7) | 1.96 | 1.58-3.00 |
| V [symbol] (mg/h/70 kg) | 585 | (6.5) | 585 | 506-676 |
| K [symbol] (μg/mL) | 7.8 | (10.7) | 7.7 | 5.2-10.9 |
| Mean transit time (h) | 0.123 | (16.6) | 0.128 | 0.059-0.249 |
| Ka [symbol] (h [symbol] ) | 0.767 | (5.2) | 0.779 | 0.567-0.917 |
| F [symbol] | 0.476 | (4.2) | 0.475 | 0.423-0.544 |

V [symbol] Covariates

| | | | | |
|---|---|---|---|---|
| Exponent for weight | 0.92 | (13.2) | 0.930 | 0.649-1.19 |
| effect | | | | |
| ALS diagnosis | 0.275 | (25.5) | 0.271 | 0.154-0.405 |

V [symbol] Covariates

| | | | | |
|---|---|---|---|---|
| ALS diagnosis | −0.360 | (17.7) | −0.350 | −0.491-−0.189 |

Inter-individual Variability **

| CL [symbol] /F | 0.807 (9.3) [9.2] | | 0.791 | 0.600-0.968 |
|---|---|---|---|---|
| V [symbol] /F | 1.131 (9.6) [11.8] | | 1.12 | 0.805-1.37 |
| V [symbol] | 0.233 (19.6) [42.4] | | 0.223 | 0.126-0.360 |
| V [symbol] | 0.180 (11.7) [15.3] | | 0.174 | 0.136-0.224 |
| Corr (CL [symbol] /F, V [symbol] /F) | 0.930 | (2.4) | 0.905 | 0.656-1.13 |

Residual Variability *

| | | | | |
|---|---|---|---|---|
| Log additive error- PB | 0.324 | (4.6) | 0.317 | 0.274-0.365 |
| Log additive error- PAA | 0.243 | (4.0) | 0.238 | 0.211-0.263 |

| | | | |
|---|---|---|---|
| Shrinkage (%) | 12.8 | — | — |

* mean estimate (relative standard error) for final model
** mean percent coefficient of variation (relative standard error) [shrinkage, %] for final model
Corr = correlation coefficient between [symbol] (relative standard error) for final model; PB = phenylbutyrate; PAA = phenylacetate; CL [symbol] /F = apparent (oral) metabolic clearance of phenylbutyrate to phenylacetate; V [symbol] /F = apparent volume of distribution of phenylbutyrate; CL [symbol] = elimination clearance of phenylacetate; V_{PAA} = volume of distribution of PAA; Ka [symbol] = PB oral absorption rate constant under fasting conditions (or on an empty stomach for ALS patient [symbol] V [symbol] = maximum rate of elimination; K [symbol] = amount at which rate of elimination of PAA is half maximal; Mean transit time- transit time from metabolism compartment to plasma compartment for PAA, represents delay in appearance of PAA in plasma; F [symbol] = relative bioavailability when phenylbutyrate is administered with food versus fasting; Ka [symbol] = oral absorption rate constant when phenylbutyrate is administered with food. [symbol]
indicates data missing or illegible when filed

### Drug Concentrations

The extensive sampling design from Protocol 35-002 allowed a complete depiction of the plasma concentration profiles in the volunteers. Following oral administration, PB was rapidly absorbed and converted to PAA. Plasma concentrations typically peaked between 0.5 to 1 hour for PB and 2 to 3 hours for PAA and fell below the lower limit of quantitation (LLOQ) by 4 to 5 hours after the dose for PB and 6 to 8 hours for PAA. Consequently, no accumulation of drug or metabolite occurred with b.i.d. dosing. Compared to fasting administration, PB plasma concentrations in the volunteers were substantially lower and profiles more prolonged following administration with a high fat meal. Food related changes in the PAA plasma concentration-time data were comparable.

The effect of food on the sparsely collected plasma concentrations from the ALS clinical trial was examined. PB administration in the clinical trial patients was classified as being administered on an empty stomach (fasting administration) when drug administration occurred ≥2 hours after or ≥1 hour before a meal. There is a trend toward higher PB and PAA plasma concentrations at 1 hour and similar or slightly lower concentrations at 4 hours was noticeable between administration of PB on an empty stomach compared with food.

PK Parameters Derived from Post Hoc Analysis

Table 25 summarizes the subject specific Cₘₐₓ and AUC_{0-∞} derived from the individual Bayesian parameter estimates in the patients with ALS.

TABLE 25

| | |
|---|---|
| Phenylbutyrate | Phenylacetate |
| Food Conditions | Food Conditions |

| | Fasting | Fed | Fasting | Fed |
|---|---|---|---|---|
| number | 53 | 41 | 53 | 41 |

Cₘₐₓ (μg/mL)

| | | | | |
|---|---|---|---|---|
| Mean | 131 | 48.4 | 24.1 | 16.6 |
| Median | 97.9 | 43.9 | 23.8 | 16.9 |
| % CV | 79.5 | 59.4 | 38.8 | 34.0 |
| 2.5th percentile | 10.3 | 8.74 | 10.1 | 8.16 |
| 97.5th percentile | 414 | 112 | 44.2 | 25.3 |
| Geometric mean | 90.9 | 39.4 | 22.3 | 15.6 |
| Geometric % CV | 126 | 79.2 | 43.7 | 37.2 |

AUC [symbol] (μg-h/mL)

| | | | | |
|---|---|---|---|---|
| Mean | 202 | 113 | 66.2 | 50.59 |
| Median | 166 | 105 | 60.0 | 47.56 |
| % CV | 78.9 | 55.3 | 49.6 | 46.2 |
| 2.5th percentile | 25.7 | 27.3 | 24.2 | 22.1 |
| 97.5th percentile | 538 | 243 | 148 | 91.2 |
| Geometric mean | 151 | 95.1 | 58.6 | 46.0 |
| Geometric % CV | 102 | 70.5 | 54.1 | 47.6 |

AUC [symbol] (μg-h/mL)

| | | | | |
|---|---|---|---|---|
| Mean | 203 | 116 | 67.2 | 51.8 |
| Median | 168 | 109 | 61.1 | 49.5 |
| % CV | 78.2 | 54.0 | 49.6 | 45.7 |
| 2.5th percentile | 28.8 | 30.3 | 25.2 | 23.2 |
| 97.5th percentile | 539 | 247 | 149 | 92.2 |
| Geometric mean | 154 | 98.8 | 58.6 | 47.0 |
| Geometric % CV | 96.7 | 67.6 | 54.1 | 46.8 |

Number- represents number of regimens receiving phenylbutyrate under fasting (>2 hours before or 1 hour after meal) or fed conditions: Cₘₐₓ = maximum plasma concentration; AUC₀₋ₗₐₛₜ = area under the plasma concentration-time curve from time zero to the last measurable plasma concentration; AUC_{0-∞} = area under the plasma concentration-time curve from time zero to infinity; % CV = coefficient of variation expressed as a percentage. [symbol]
indicates data missing or illegible when filed

### Example 4: TUDCA Pharmacokinetics Study

A study was carried out to examine plasma concentration data in various subgroups for the protocol AMX3500 Phase 2 study. This example summarizes plasma concentrations of TUDCA and 2 metabolites, also related acids, UDCA, the de-conjugated TUDCA metabolite, and GUDCA, the glycine conjugate of UDCA.

For each of the 2 treatment arms (treatment and placebo), subjects were further divided into 2 sample sequence groups for schedule of pharmacokinetic measurements:
1. 1 hour post-dose for the Week 12 visit and 4 hours post-dose for the Week 24 visit
2. 4 hours post-dose for the Week 12 visit and 1 hour post-dose for the Week 24 visit

Antibiotics listed in concomitant medications were the basis for comparing subjects who took antibiotics overlapping the time of their visit to those who did not. Twenty-four distinct medication descriptions were identified as containing antibiotics. Dates of antibiotic use were compared to dates of visits and if the antibiotic use date range contained the visit date, then that visit was flagged for that subject's data for antibiotic use. This approach does not, however, flag subjects who took antibiotics before the visit but whose administration of antibiotics ended before the visit occurred, so may not account for some antibiotic with long elimination half-life. These antibiotics could hypothetically be in a subject's body still if this event occurred. However, it was observed that only a small number of subjects took antibiotics near to visit dates but not overlapping visit dates. In other cases, subjects were taking antibiotics on the date of one visit but not the others. Some subjects are therefore in both categories of antibiotic use and nonuse, though no individual visits for a subject are counted in both categories at the same time.

Subjects whose race were recorded as “Black” or “African American” were evaluated according to the standard Glomerular Filtration Rate (eGFR) rating for that race. The other standard eGFR rating used, sometimes referred to as a standard for people who are White, was applied to subjects of all other races. These rates were analyzed as continuous in mL/min/1.73 m2 units and as the following categories:
1. Less than 90 mL/min/1.73 m²
2. Equal to or greater than 90 mL/min/1.73 m²

Age was converted from a continuous variable to 2 categories:
1. As old or older than 65 years old
2. Younger than 65 years old

All boxplots boxes are bounded at the 25th and 75th percentile, with whiskers at 10th and 90^{th} percentiles, outliers beyond that as points, the black line as median, and red line as mean.

### Results

The results show that TUDCA, UDCA, and GUDCA plasma concentrations appear to have reached steady state by Week 12 supporting the pooling plasma concentration data across study visits. Steady-state plasma concentrations of TUDCA. UDCA, and GUDCA after administration of AMX0035 in ALS patients are very highly variable and generally at least an order of magnitude higher than endogenous levels, which are also highly variable.

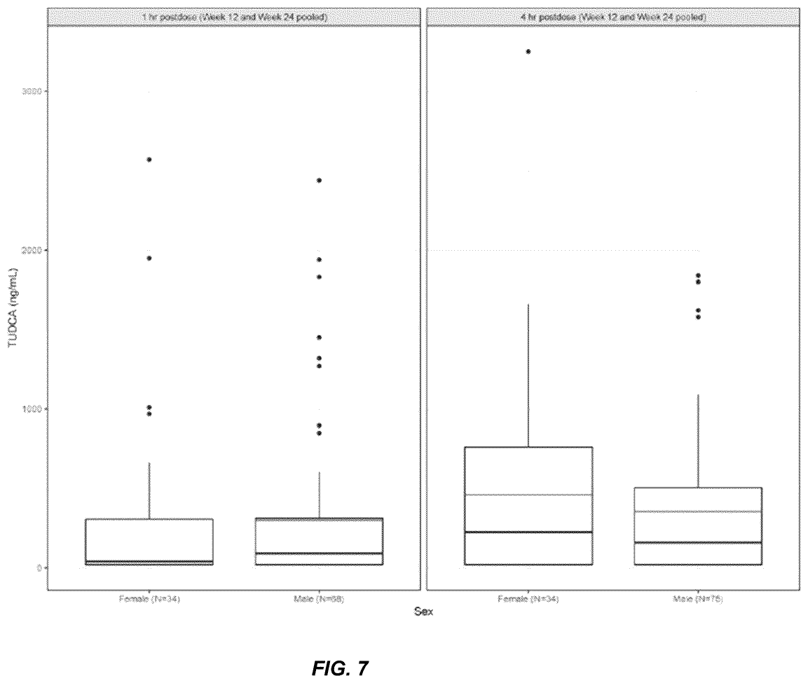

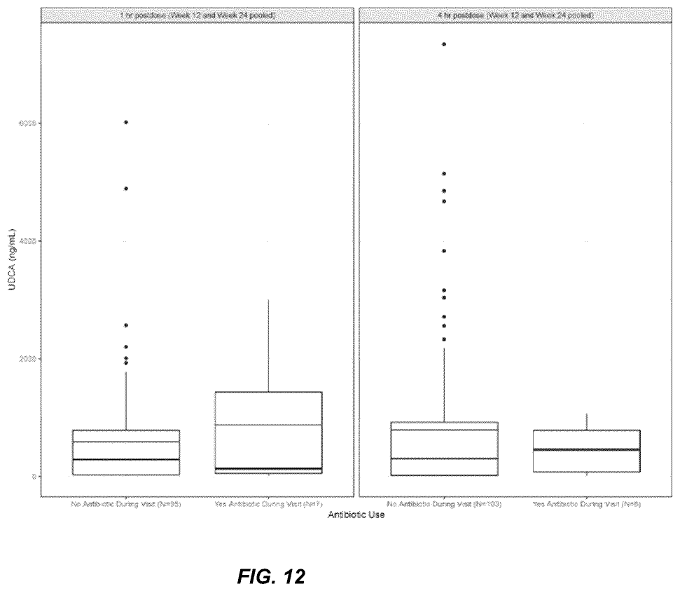

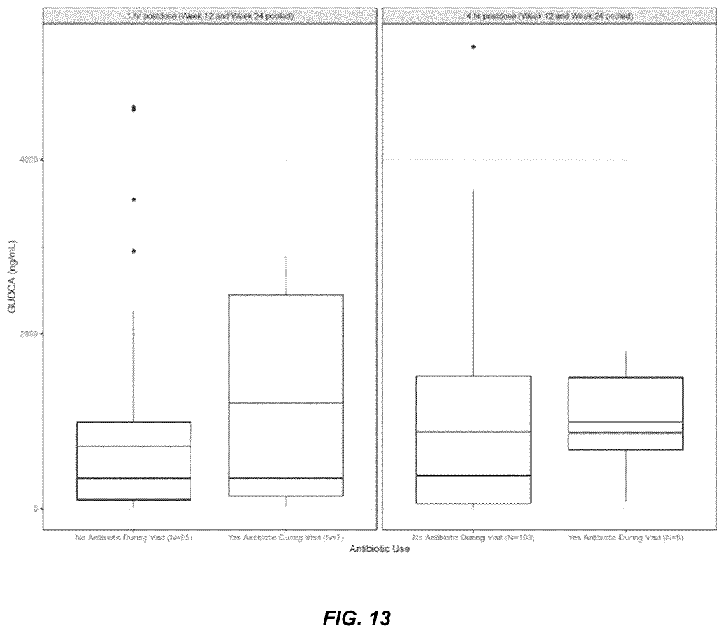

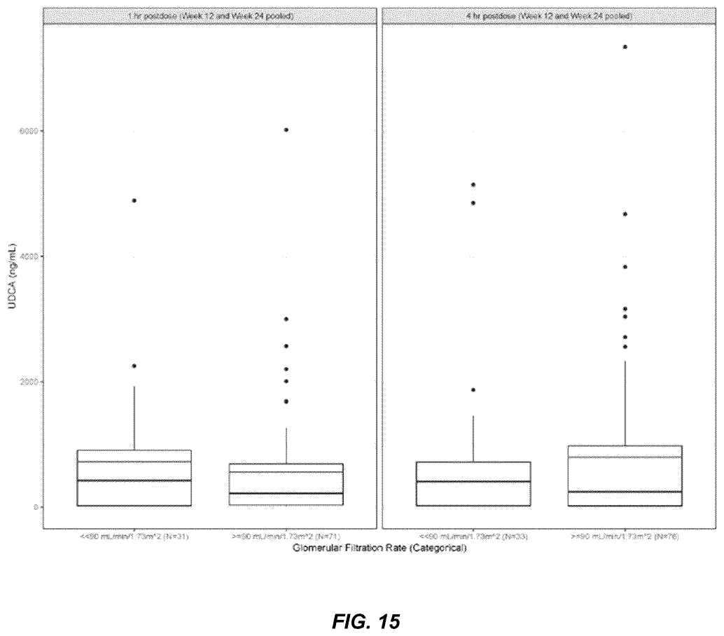

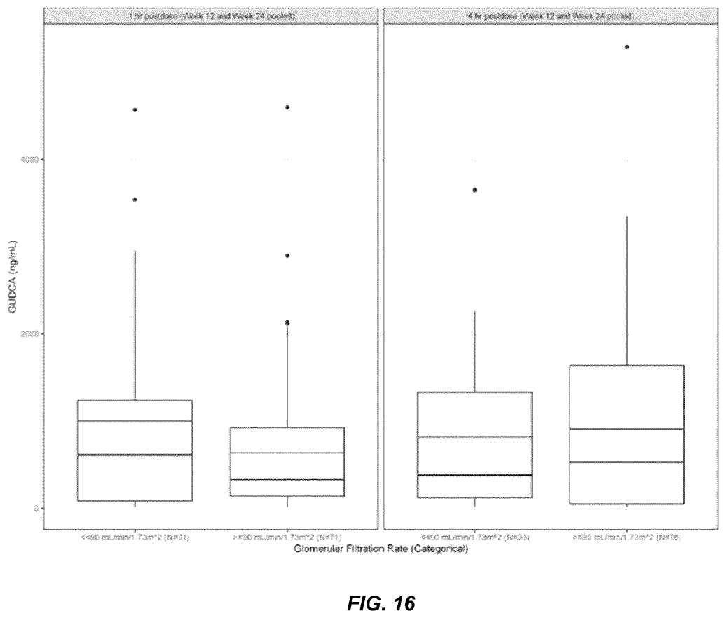

The mean-to-standard-deviation ratio (MSDR) is a simple metric for examining the variability in each group and comparing across acids and groups. Table 26 shows MSDR of TUDCA levels for the AMX0035 arm for 1-hour post-dose comparing 12 and 24 week groups (82.10% vs. 66.3%) and the 4-hour post-dose groups (97.7% vs. 112.7%), with noted high variability for all of these groups. The same pattern of high variability is found for UDCA (86.7% vs. 89.6%, 102.7% vs. 69.1%)(Table 27) and for GUDCA (106.7% vs. 105.9%, 138.1% vs. 134.3%) (Table 28). It can be noted that significant endogenous concentrations of UDCA and GUDCA are present in patients treated with placebo. Pooled groups by hours postdose have similar patterns of high variability (Table 29). FIGS. 7-26 demonstrate that outliers contribute to the high variability found in the tables.

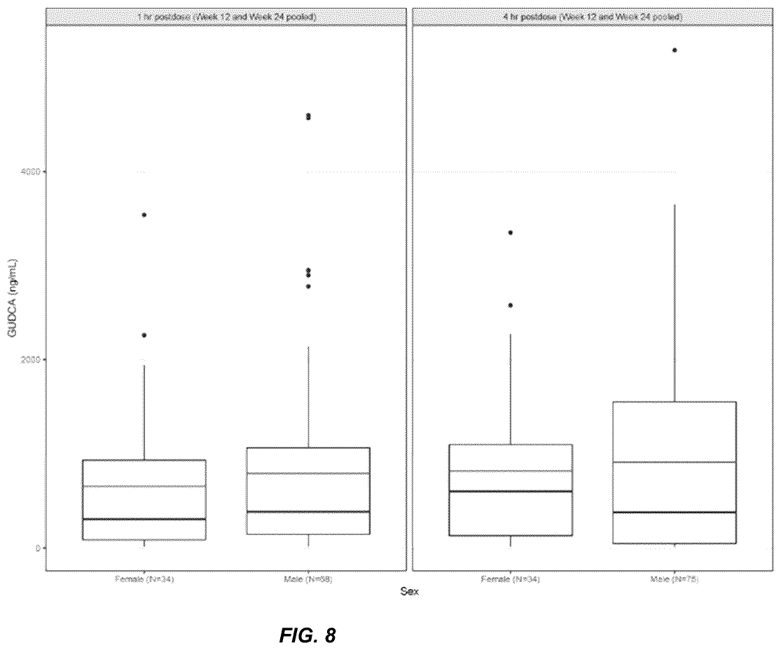

FIG. 7 shows TUDCA levels of 4-hour post-dose female subjects have a more positively skewed distribution, with 75th and 90th percentiles much higher than the other 3 groups. All 4 groups in FIG. 7 have similar distributions of UDCA levels. FIG. 8 shows 3 groups with similar distributions of GUDCA levels, but male subjects in the 4-hour post-dose group having more variability and higher 75th and 90th percentile scores.

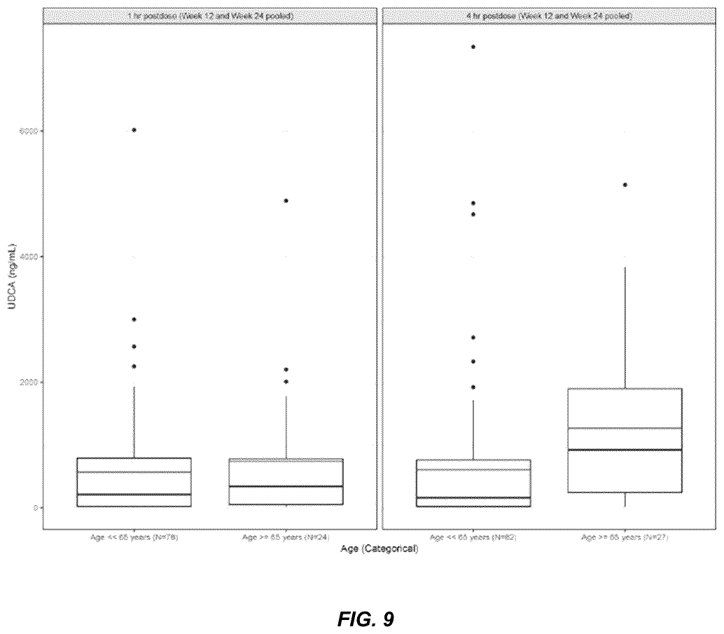

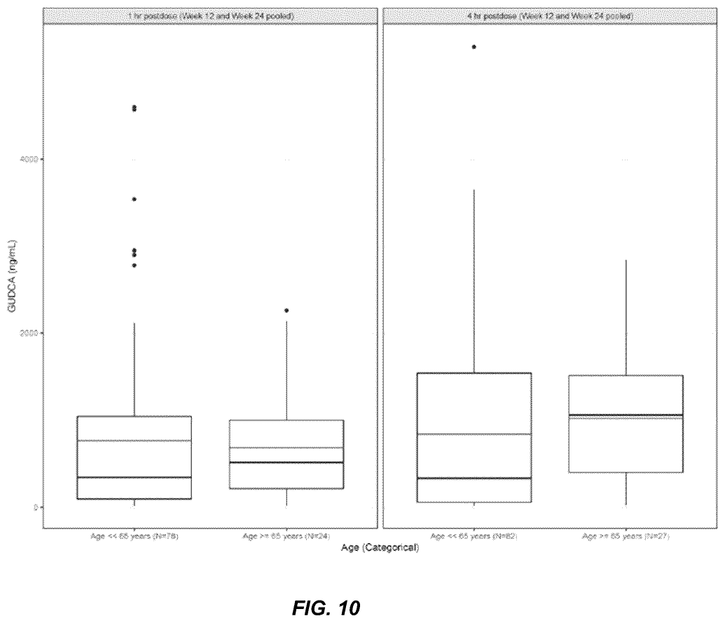

With age categories (1 hr post-dose (age <<65 tears vs age >=65 years) and 4 hr post-dose (age <<65 tears vs age >=65 years), TUDCA levels show similar distribution patterns. In UDCA, the 65-years-old and over group for 4 hours post-dose show a different pattern from the other 3 groups, with higher variability and higher levels overall, as shown in FIG. 9. GUDCA levels in FIG. 10 show similar distribution patterns among 3 groups, but the under 65-years-old group has more variability and a lower median compared to the over 65-years-old 4-hour post-dose group.

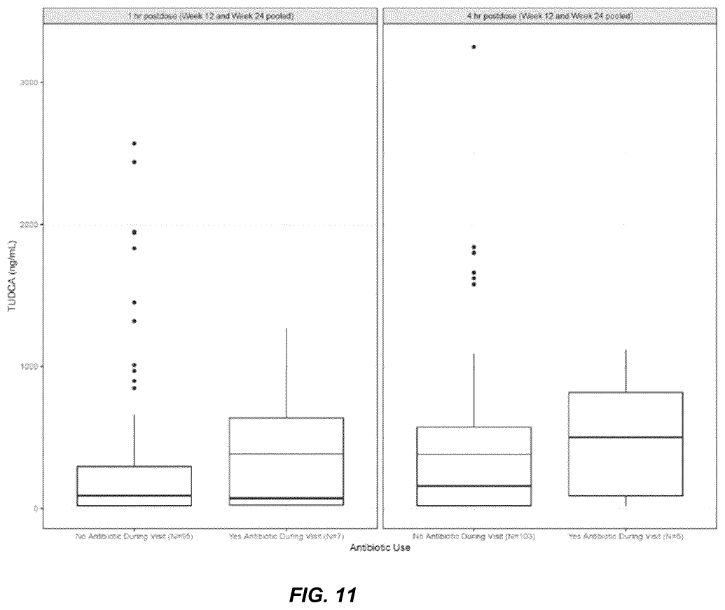

For each acid, mean levels are comparable across each group in the antibiotic use figures. Distributions, however, sometimes have higher medians and variability in TUDCA, UDCA, and GUDCA (FIGS. 11, 12, and 13 respectively). These higher median groups are composed of subject who used antibiotics.

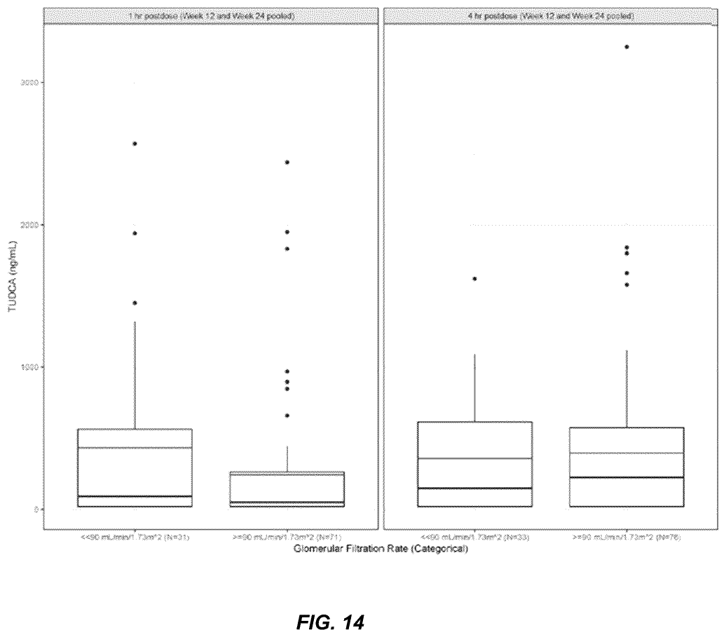

The high GFR group (greater than 90 mL/min/1.73 m²) for 1-hour post-dose has a narrower distribution with lower plasma concentrations than the other 3 groups when inspecting visually the boxplots found for TUDCA, UDCA, and GUDCA in FIG. 14-16.

TABLE 26
TUDCA Plasma Concentration (ng/mL) Summary Statistics per Visit

| Visit | Statistic | AMX0035 | | Placebo | |
|---|---|---|---|---|---|
| Baseline | N | 77 | | 44 | |
| Visit | Mean [symbol] g/mL (SD [symbol] g/mL) | 39.7 | (82.93) | 20.4 | (2.44) |
| | Median [symbol] g/mL (IQR [symbol] g/mL) | 20.0 | (0.0) | 20.0 | (0.0) |
| | (Q1 [symbol] g/mL, Q3 [symbol] g/mL) | (20.0, 20.0) | | (20.0, 20.0) | |
| | Min [symbol] g/mL, Max [symbol] g/mL | 20.0, 577.0 | | 20.0, 36.2 | |
| | Mean to Standard Deviation Ratio (%) | 47.8% | | 834.0% | |
| Early | N | 7 | | 3 | |
| Discontinuation | Mean [symbol] g/mL (SD [symbol] g/mL) | 266.0 | (597.77) | 20.0 | (0.00) |
| | Median [symbol] g/mL (IQR [symbol] g/mL) | 20.0 | (65.5) | 20.0 | (0.0) |
| | (Q1 [symbol] g/mL, Q3 [symbol] g/mL) | (20.0, 85. [symbol] ) | | (20.0, 20.0) | |
| | Min [symbol] g/mL, Max [symbol] g/mL | 20.0, 1620.0 | | 20.0, 26.0 | |
| | Mean to Standard Deviation Ratio (%) | 44.5% | | % | |
| Week 12 - | N | 36 | | 17 | |
| 1 hr Post-dose | Mean [symbol] g/mL (SD [symbol] g/mL) | 502.8 | (612.15) | 20.0 | (0.00) |
| | Median [symbol] g/mL (IQR [symbol] g/mL) | 248.5 | (677.3) | 20.0 | (0.0) |
| | (Q1 [symbol] g/mL, Q3 [symbol] g/mL) | (76.8, 754.0) | | (20.0, 20.0) | |
| | Min [symbol] g/mL, Max [symbol] g/mL | 20.0, 2440.0 | | 20.0, 26.0 | |
| | Mean to Standard Deviation Ratio (%) | 82.1% | | % | |
| Week 12 - | N | 36 | | 22 | |
| 4 hrs Post-dose | Mean [symbol] g/mL (SD [symbol] g/mL) | 636.5 | (651.63) | 20.6 | (1.78) |
| | Median [symbol] g/mL (IQR [symbol] g/mL) | 445.6 | (587.5) | 20.0 | (0.0) |
| | (Q1 [symbol] g/mL, Q3 [symbol] g/mL) | (251.0, 838.5) | | (20.0, 20.0) | |
| | Min [symbol] g/mL, Max [symbol] g/mL | 20.0, 3250.0 | | 20.0, 27.8 | |
| | Mean to Standard Deviation Ratio (%) | 97.7% | | 1150% | |
| Week 24 - | N | 31 | | 18 | |
| 1 hr Post-dose | Mean [symbol] g/mL (SD [symbol] g/mL) | 383.2 | (378.26) | 20.4 | (1.53) |
| | Median [symbol] g/mL (IQR [symbol] g/mL) | 180.0 | (286.3) | 20.0 | (0.0) |
| | (Q1 [symbol] g/mL, Q3 [symbol] g/mL) | (89.7, 376.0) | | (20.0, 20.0) | |
| | Min [symbol] g/mL, Max [symbol] g/mL | 20.0, 2570.0 | | 20.0, 26.5 | |
| | Mean to Standard Deviation Ratio (%) | 66.3% | | 1329% | |
| Week 24 - | N | 34 | | 18 | |
| 4 hrs Post-dose | Mean [symbol] g/mL (SD [symbol] g/mL) | 491.7 | (436.06) | 1 [symbol] 0.2 | (248.54) |
| | Median [symbol] g/mL (IQR [symbol] g/mL) | 401.0 | (606.0) | 20.0 | (0.0) |

| | | |
|---|---|---|
| (Q1 [symbol] g/mL, Q3 [symbol] g/mL) | (148.0, 754.0) | (20.0, 20.0) |
| Min [symbol] g/mL, Max [symbol] g/mL | 20.0, 1620.0 | 20.0, 79 [symbol] .0 |
| Mean to Standard Deviation Ratio (%) | 112.7% | 48.4% |

[symbol]

indicates data missing or illegible when filed

TABLE 27
UDCA Plasma Concentration (ng/mL) Summary Statistics per Visit

| Visit | Statistic | AMX0035 | | Placebo | |
|---|---|---|---|---|---|
| Baseline | N | 77 | | 44 | |
| Visit | Mean [symbol] g/mL (SD [symbol] g/mL) | 137.9 | (682.55) | 66.3 | (110.66) |
| | Median [symbol] g/mL (IQR [symbol] g/mL) | 20.0 | (32.5) | 20.0 | (45.2) |
| | (Q1 [symbol] g/mL, Q3 [symbol] g/mL) | (20.0, 52.5) | | (20.0, 65.2) | |
| | Min [symbol] g/mL, Max [symbol] g/mL | 20.0, 5970.0 | | 20.0, 587.0 | |
| | Mean to Standard Deviation Ratio (%) | 25.2% | | 59.9% | |
| Early | N | 7 | | 3 | |
| Discontinuation | Mean [symbol] g/mL (SD [symbol] g/mL) | 354.7 | (783.53) | 31.6 | (20.15) |
| | Median [symbol] g/mL (IQR [symbol] g/mL) | 20.4 | (240.0) | 20.0 | (34.9) |
| | (Q1 [symbol] g/mL, Q3 [symbol] g/mL) | (20.0, 260.0) | | (20.0, 54.9) | |
| | Min [symbol] g/mL, Max [symbol] g/mL | 20.0, 2120.0 | | 20.0, 54.9 | |
| | Mean to Standard Deviation Ratio (%) | 45.3% | | 157.0% | |
| Week 12 - | N | 36 | | 17 | |
| 1 hr Post-dose | Mean [symbol] g/mL (SD [symbol] g/mL) | 7 [symbol] .0 | (883.24) | 32.8 | (23.25) |
| | Median [symbol] g/mL (IQR [symbol] g/mL) | 591.5 | (639.0) | 20.0 | (16.9) |
| | (Q1 [symbol] g/mL, Q3 [symbol] g/mL) | (270.5, 909.5) | | (20.0, 36.9) | |
| | Min [symbol] g/mL, Max [symbol] g/mL | 25.6, 4890.0 | | 20.0, 106.0 | |
| | Mean to Standard Deviation Ratio (%) | 85.7% | | 140.9% | |
| Week 12 - | N | 36 | | 22 | |
| 4 hrs Post-dose | Mean [symbol] g/mL (SD [symbol] g/mL) | 1299.1 | (1265.39) | 46.4 | (51.73) |
| | Median [symbol] g/mL (IQR [symbol] g/mL) | 919.5 | (1250.5) | 20.0 | (16.4) |
| | (Q1 [symbol] g/mL, Q3 [symbol] g/mL) | (414.5, 1665.0) | | (20.0, 36.4) | |
| | Min [symbol] g/mL, Max [symbol] g/mL | 20.0, 4850.0 | | 20.0, 182.0 | |
| | Mean to Standard Deviation Ratio (%) | 102.7% | | 89.7% | |
| Week 24 - | N | 31 | | 18 | |
| 1 hr Post-dose | Mean [symbol] g/mL (SD [symbol] g/mL) | 1077.0 | (1202.37) | 39.2 | (36.63) |
| | Median [symbol] g/mL (IQR [symbol] g/mL) | 614.0 | (1388.0) | 20.0 | (18.9) |
| | (Q1 [symbol] g/mL, Q3 [symbol] g/mL) | (302.0, 1690.0) | | (20.0, 38.9) | |
| | Min [symbol] g/mL, Max [symbol] g/mL | 20.0, 6020.0 | | 20.0, 159.0 | |
| | Mean to Standard Deviation Ratio (%) | 89.6% | | 107.1% | |
| Week 24 - | N | 34 | | 18 | |
| 4 hrs Post-dose | Mean [symbol] g/mL (SD [symbol] g/mL) | 1031.6 | (1491.09) | 67.6 | (128.42) |
| | Median [symbol] g/mL (IQR [symbol] g/mL) | 614.0 | (754.0) | 20.0 | (50.1) |

| | | |
|---|---|---|
| (Q1 [symbol] g/mL, Q3 [symbol] g/mL) | (240.0, 994.0) | (20.0, 70.1) |
| Min [symbol] g/mL, Max [symbol] g/mL | 20.0, [symbol] 340. [symbol] | 20.0, 563.0 |
| Mean to Standard Deviation Ratio (%) | 69.1% | 52.7% |

[symbol]

indicates data missing or illegible when filed

TABLE 28
GUDCA Plasma Concentration (ng/mL) Summary Statistics per Visit

| Visit | Statistic | AMX0035 | | Placebo | |
|---|---|---|---|---|---|
| Baseline | N | 77 | | 44 | |
| Visit | Mean [symbol] g/mL (SD [symbol] g/mL) | 208.9 | (540.96) | 135.0 | (172.80) |
| | Median [symbol] g/mL (IQR [symbol] g/mL) | 70.8 | (151.5) | 66.7 | (123.6) |
| | (Q1 [symbol] g/mL, Q3 [symbol] g/mL) | (26.5, 178.0) | | (28.0, 151.5) | |
| | Min [symbol] g/mL, Max [symbol] g/mL | 20.0, 4540.0 | | 20.0, 826.6 | |
| | Mean to Standard Deviation Ratio (%) | 38.6% | | 78.1% | |
| Early | N | 7 | | 3 | |
| Discontinuation | Mean [symbol] g/mL (SD [symbol] g/mL) | 434.5 | (621.20) | 54.7 | (33.12) |
| | Median [symbol] g/mL (IQR [symbol] g/mL) | 150.0 | ( [symbol] 05.0) | 56.0 | (66.2) |
| | (Q1 [symbol] g/mL, Q3 [symbol] g/mL) | (72.0, 777.0) | | (21.0, 87.2) | |
| | Min [symbol] g/mL, Max [symbol] g/mL | 22.3, 1720.0 | | 21.0, 87.2 | |
| | Mean to Standard Deviation Ratio (%) | 69.9% | | 165.3% | |
| Week 12 - | N | 36 | | 17 | |
| 1 hr Post-dose | Mean [symbol] g/mL (SD [symbol] g/mL) | 1023.1 | (959.31) | 98.5 | (94.07) |
| | Median [symbol] g/mL (IQR [symbol] g/mL) | 773.0 | (1115.0) | 74.3 | (123.9) |
| | (Q1 [symbol] g/mL, Q3 [symbol] g/mL) | (345.0, 1460.0) | | (30.1, 154.0) | |
| | Min [symbol] g/mL, Max [symbol] g/mL | 20.0, 4570.0 | | 20.0, 394.0 | |
| | Mean to Standard Deviation Ratio (%) | 106.7% | | 104.7% | |
| Week 12 - | N | 36 | | 22 | |
| 4 hrs Post-dose | Mean [symbol] g/mL (SD [symbol] g/mL) | 1513.8 | (1096.25) | 129.9 | (120. [symbol] 5) |
| | Median [symbol] g/mL (IQR [symbol] g/mL) | 1505.0 | (1436.5) | 106.5 | (113.8) |
| | (Q1 [symbol] g/mL, Q3 [symbol] g/mL) | (673.5, 2110.0) | | (47.2, 161.0) | |
| | Min [symbol] g/mL, Max [symbol] g/mL | 20.0, 5290.0 | | 20.0, 521.0 | |
| | Mean to Standard Deviation Ratio (%) | 138.1% | | 108.2% | |
| Week 24 - | N | 31 | | 18 | |
| 1 hr Post-dose | Mean [symbol] g/mL (SD [symbol] g/mL) | 1133.7 | (1070.32) | 142.2 | (200.64) |
| | Median [symbol] g/mL (IQR [symbol] g/mL) | 815.0 | (1424.0) | 77.5 | (111.9) |
| | (Q1 [symbol] g/mL, Q3 [symbol] g/mL) | (336.0, 1760.0) | | (46.1, 158.0) | |
| | Min [symbol] g/mL, Max [symbol] g/mL | 25.5, 4600.0 | | 20.0, 882.0 | |
| | Mean to Standard Deviation Ratio (%) | 105.9% | | 70 [symbol] % | |
| Week 24 - | N | 34 | | 18 | |
| 4 hrs Post-dose | Mean [symbol] g/mL (SD [symbol] g/mL) | 1113.2 | (828.90) | 58.2 | (43.87) |
| | Median [symbol] g/mL (IQR [symbol] g/mL) | 10 [symbol] 0.0 | (1181.0) | 42.9 | (58.6) |

| | | |
|---|---|---|
| (Q1 [symbol] g/mL, Q3 [symbol] g/mL) | (369.0, 1550.0) | (23.2, 81.8) |
| Min [symbol] g/mL, Max [symbol] g/mL | 20.0, 3350.0 | 20.0, 155.0 |
| Mean to Standard Deviation Ratio (%) | 134.3% | 133.2% |

[symbol]

indicates data missing or illegible when filed

TABLE 29
Plasma Concentration (ng/mL) Summary Statistics per Pooled Visit for Subjects in AMX0035 Arm

| Pooled Vist for AMX0035 Treatment Group | Statistic | TUDCA | | UDCA | | GUDCA | |
|---|---|---|---|---|---|---|---|
| Predose | N | 77 | | 77 | | 77 | |
| (Baseline Visit) | Mean [symbol] g/mL (SD [symbol] g/mL) | 39.7 | (82.93) | 137.9 | (682.55) | 208.9 | (540.96) |
| | Median [symbol] g/mL (IQR [symbol] g/mL) | 20.0 | (0.0) | 20.0 | (32.5) | 70.8 | (151.5) |
| | (Q1 [symbol] g/mL, Q3 [symbol] g/mL) | (20.0, 20.0) | | (20.0, 52.5) | | (26.5, 178.0) | |
| | Min [symbol] g/mL, Max [symbol] g/mL | 20.0, 577.0 | | 20.0, 5970.0 | | 20.0, 4540.0 | |
| | Mean to Standard Deviation Ratio (%) | 47.8% | | 20.2% | | 38.5% | |
| 1 hr Post-dose | N | 67 | | 67 | | 67 | |
| (Week 12 and Week 24 Pooled) | Mean [symbol] g/mL (SD [symbol] g/mL) | 447.5 | (595.25) | 909.9 | (1046.64) | 1074.3 | (1 [symbol] 05.89) |
| | Median [symbol] g/mL (IQR [symbol] g/mL) | 208.0 | (448.3) | 614.0 | (832.6) | 815.0 | (1287.0) |
| | (Q1 [symbol] g/mL, Q3 [symbol] g/mL) | (89.7, 538.0) | | (283.0, 1120.0) | | (343.0, 1630.0) | |
| | Min [symbol] g/mL, Max [symbol] g/mL | 20.0, 2570.0 | | 20.0, 6020.0 | | 20.0, 4600.0 | |
| | Mean to Standard Deviation Ratio (%) | 75.2% | | 86.9% | | 106.8% | |
| 4 hrs Post-dose | N | 70 | | 70 | | 70 | |
| (Week 12 and Week 24 Pooled) | Mean [symbol] g/mL (SD [symbol] g/mL) | 566.1 | (558.25) | 1168.9 | (1376.14) | 1319.2 | (989.37) |
| | Median [symbol] g/mL (IQR [symbol] g/mL) | 419.0 | (623.0) | 779.0 | (1079.0) | 1190.0 | (1376.0) |

| | | | |
|---|---|---|---|
| (Q1 [symbol] g/mL, Q3 [symbol] g/mL) | (158.0, 781.0) | (311.0, 1390.0) | (534.0, 1910.0) |
| Min [symbol] g/mL, Max [symbol] g/mL | 20.0, 3250.9 | 20.0, 7340.0 | 20.0, 5290.0 |
| Mean to Standard Deviation Ratio (%) | 101.4% | 84.9% | 133.3% |

[symbol]

indicates data missing or illegible when filed

### Example 5: Statistical Analysis of Bile Acids in PK Samples Derived from Patients with ALS from the CENTAUR Trial

The goal of the study is to evaluate the effect of AMX0035 on human plasma bile acids profiles. TUDCA being a bile acid that can be produced endogenously in humans, its impact on the balance of the bile acids pathway can be an important factor in both the efficacy and potential toxicity of the treatment. A targeted metabolomics approach was applied using the biocrates AbsoluteIDQ® Bile Acids assay to measure 20 bile acids by LC-MS.

The following samples were used:
The primary analysis, or Complete cases (CC), comprised exclusively samples from subjects from whom all 3 timepoints (Baseline Visit, Week 12 and Week 24) were available and where no early discontinuation occurred.
The secondary analysis, or Partial cases (PC), included samples from subjects with at least 2 timepoints available, i.e. Baseline Visit and Week 12 or Week 24, or all 3 timepoints. This analysis also included samples taken upon early discontinuation (ED), either at Week 12 or Week 24, in order to study potentially relevant undesired effects of the treatment.
The Time course dataset (TC, 270 samples) comprised the same samples as the CC dataset analyzed with a different group category to investigate the interaction of treatment and treatment duration.
The Strength of response dataset (SR, 108 samples) comprised a subset of the CC dataset selected on the basis of their ALSFRS-R slope.
The SRTC dataset comprised the same samples as the SR dataset analyzed with a different group category to investigate the interaction of strength of response and treatment duration.

### Univariate Statistics

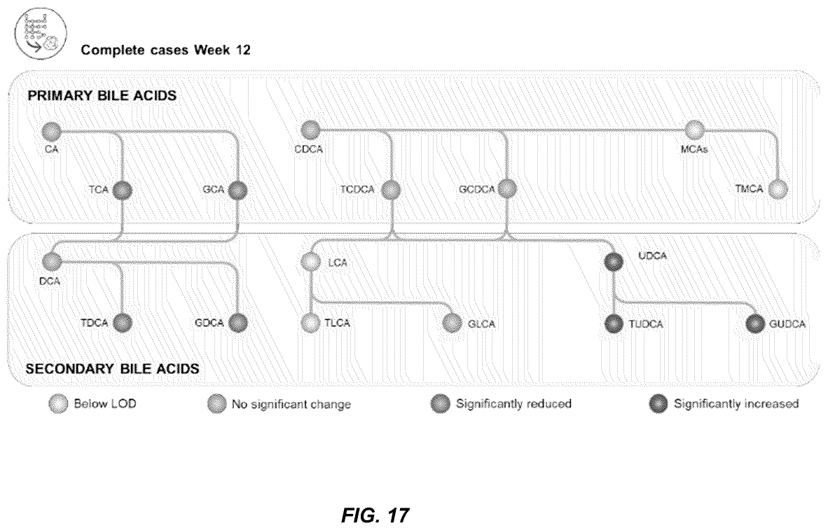

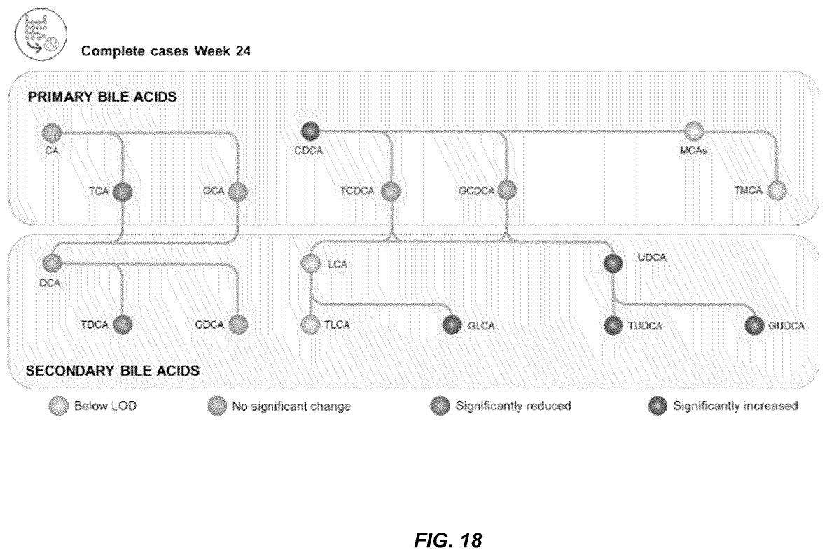

In the primary analysis of Complete cases, ANOVA models were used to compare AMX0035 and placebo groups at the three time points: Baseline Visit, Week 12 and Week 24. FIGS. 17 and 18 show a pathway visualization of the effects of AMX0035 treatment at Week 12 and Week 24. There were no significant differences between the two groups at Baseline.

At both Weeks 12 and 24, TUDCA, UDCA and GUDCA, as well as the hydrophilic/hydrophobic bile acids ratio were increased in AMX0035-treated subjects, most likely as a direct effect of the oral administration of TUDCA to these subjects. Indeed, UDCA, although a precursor of TUDCA under physiological conditions, can also be produced by deconjugation of taurine from TUDCA in the intestine. In addition, UDCA is considered to be exclusively produced by intestinal microbiota, which further supports its synthesis from orally administered TUDCA in these conditions. Finally, after taurine deconjugation, UDCA can undergo reconjugation to glycine to form GUDCA during the enterohepatic recycling of TUDCA. Interestingly, it was UDCA and not TUDCA that was found to be negatively correlated, albeit weakly, to the ALSFRS-R slope in the correlation analysis (Table 30). This suggests high UDCA values could be expected in subjects with rapid disease progression, while low UDCA values would be expected in subjects with slow disease progression.

At Week 12, glycine and taurine conjugates (GCA (Glycocholic acid), TCA (Taurocholic acid), GDCA (Glycodeoxycholic acid), TCDA) and indicators of glycine and taurine conjugation of CDCA (Chenodeoxycholic acid), DCA (Deoxycholic acid) and pBAs were all decreased in AMX0035-treated subjects. A similar trend was still present at Week 24, with the exception of glycine conjugates GCA and GDCA that were still globally decreased but not significantly. The pBA conjugation ratio, reflecting both glycine and taurine conjugation of CA and CDCA, was also decreased at both time points. Interestingly, at Week 12 the sum of conjugated primary bile acids was significantly decreased, while at Week 24, it was the sum of unconjugated primary bile acids that was increased.

The levels of CA (Cholic acid) and CDCA were globally decreased and increased, respectively, although the difference was only significant for CDCA at Week 24. The CDCA/CA ratio, however, was significantly increased at both time points. The biosynthesis of bile acids is classically described as the combined effects of a classical pathway producing CA and CDCA, and an alternative pathway favoring CDCA production.

At Week 12, the sum of 12-alpha-hydroxylated bile acids (CA, DCA and their conjugates) was decreased. An increase in this indicator has been associated with insulin resistance. Even though a decrease was observed here, this could denote an effect related to diabetes, which was one of the confounders included in the analysis. At Week 24, the sum of glycine conjugated bile acids was significantly increased, possibly as a consequence of the more marked increase in GLCA at this timepoint.

Overall in CC, the AMX0035 treatment (including a daily dose of TUDCA) caused an increase in TUDCA and related metabolites and indicators, both at Weeks 12 and 24. Interestingly, CA and DCA downstream metabolites conjugated to glycine and taurine were decreased at both time points, possibly due to a shift in metabolic resources to accommodate the high levels of TUDCA and GUDCA. Similarly, an increase in the UDCA precursor CDCA could be related to the access of UDCA caused by the treatment.

Additional information from PC: This dataset differs from CC by the fact that it includes, in addition to the 270 CC samples, samples from subjects that only participated up to 12 weeks, as well as subjects who provided samples at Week 24 but were forced to discontinue the treatment. The global profile, as shown in the univariate statistics file, was similar to that of CC. The few differences are discussed below.

In PC, the trend of increased CDCA levels in AMX0035-treated subjects was significant from Week 12 already and continued to Week 24. The sum of conjugated pBAs was also decreased, but not significantly, while the increase in the sum of glycine conjugated bile acids was here significant while it was slightly above the p-value threshold in CC. A unique feature of PC at Week 12 was the significant decrease of the TDCA synthesis from CA ratio (TDCA/CA), although here again the trend was present but not significant in other comparisons.

At Week 24, the main difference to CC was that the effects on GCA, GDCA and the sum of 12-alpha-hydroxylated bile acids (all decreased) were sustained from Week 12, while they had dissipated by Week 24 in CC.

Time course analysis (TC): This analysis focused on the influence of time (or here treatment duration) on the effect of the treatment. To this end, the CC dataset was utilized with a different “group” category combining all AMX0035 or Placebo samples, regardless of the timepoint. The influence of the different timepoints was then studied in dedicated interaction tests.

Three interaction effects were studied here: (i) the interaction between treatment and timepoint between Baseline and Week 12, (ii) the interaction between treatment and timepoint between Baseline and Week 24, and (iii) the interaction between treatment and timepoint between Week 12 and Week 24.

Here again, increased TUDCA, UDCA and GUDCA levels, as well as an increased hydrophilic/hydrophobic bile acids ratio were the most striking effects when studying the interaction between treatment and time between later timepoints and Baseline. The increase in CDCA/CA ratio was also robustly present when comparing to Baseline, as well as the decrease in glycine and taurine conjugates and related indicators.

Interestingly, the decrease in the TDCA/CA ratio was significant for interactions i and ii. In CC, a trend towards a decrease was present at both Week 12 and Week 24, but not significant. This suggests an overall role of TDCA synthesis from CA by the gut microbiome that could have been overlooked in the simple ANOVA analysis. Interestingly, this ratio was also decreased at Week 12 in the PC analysis. This ratio strongly associated with cognitive decline in a study of serum bile acids profiles in Alzheimer s disease.

This time course analysis revealed, however, that there was no interaction effect of treatment and timepoint between Week 12 and Week 24. Thus, the effects of the AMX0035 treatment on bile acids levels were globally sustained between these two timepoints.

### Correlation Analysis

Correlation of metabolites concentrations and metabolism indicators with the parameter “ALSFRS-R slope” were investigated for the most relevant sub-groups of the CC dataset.

ALSFRS-R (ALS functional rating scale—revised) is a broadly used indicator of disease progression in ALS patients. Here, the slope over the 24-week study was used after correction and transformation to allow correlation analysis. The higher the ALSFRS-R slope, the slower the progression of the disease over the 24-week period. Thus, a positive correlation with ALSFRS-R slope indicates that the value (metabolite or indicator) was high in patients with slower disease progression, and low in patients with rapid disease progression. Inversely, a negative correlation with ALSFRS-R slope corresponds to a high value in patients with rapid disease progression, and a low value in patients with slow disease progression.

TABLE 30
Metabolites/indicators significantly correlated
with transformed ALSFRS-R slope in AMX0035 group

| Metabolite/Indicator | Pearson correlation | p-value | FDR |
|---|---|---|---|
| CDCA | −0.351 | 0.005 | 0.053 |
| UDCA | −0.317 | 0.013 | 0.074 |
| Glycine Conj of CDCA | 0.298 | 0.020 | 0.074 |
| Glycine Conj of DCA | 0.269 | 0.036 | 0.123 |
| Glycine Conj of pBA | 0.300 | 0.019 | 0.074 |
| Conj pBA_Unconj pBA | 0.312 | 0.014 | 0.074 |
| Sum Unconj BA | −0.300 | 0.019 | 0.074 |
| Sum Unconj pBA | −0.347 | 0.006 | 0.053 |
| Tau Conj of CA | 0.255 | 0.047 | 0.146 |
| Tau Conj of CDCA | 0.381 | 0.002 | 0.052 |
| Tau Conj of pBA | 0.373 | 0.003 | 0.052 |

Correlation analyses were performed, using different sub-groups of the CC dataset at timepoint Week 24 vs. transformed ALSFRS-R slope. The only sub-group showing correlation with the disease progression indicator was the full group of AMX0035-treated patients at Week 24 (n=61). As shown in Table 30 above, two metabolites had a weak negative correlation to ALSFRS-R slope (CDCA and UDCA). Metabolism indicators related to glycine and taurine conjugation showed weak positive correlations and the sums of unconjugated (primary) bile acids were weakly negatively correlated to ALSFRS-R slope.

When separating this group into sub-groups based on other ALS medication (Edaravone, Riluzole, both or none), no metabolite/indicator was significantly correlated with the transformed ALSFRS-R slope. Please note that for these sub-groups the number of replicates was very low (maximum n=23 in the Riluzole only group). A similar evaluation in samples from Placebo subjects at Week 24 showed no significant correlation to ALSFRS-R slope for the entire set (n=29) and for ALS treatment sub-groups (again with very low replicate numbers).

### Strength of Response

When separating the AMX0035-treated subjects into strength of response groups based on ALSFRS-R slope, differences could be observed in the ANOVA results between Strong and Weak responders at Week 24 only. CDCA and TDCA were the only metabolites that could distinguish the two groups at this timepoint. CDCA, which is produced via both the classical and alternative bile acid biosynthesis pathways, but also a precursor of UDCA, was higher in strong responders. TDCA and the TDCA/CA ratio that was found to associate with cognitive decline were both higher in Weak responders.

Interestingly, the levels of TUDCA, UDCA, GUDCA or the hydrophilic/hydrophobic bile acids ratio did not appear to be a critical factor to differentiate these two sub-groups. Strong responders had significantly lower ratios for processes related to glycine and taurine conjugation, and higher levels of unconjugated bile acids and unconjugated pBAs. In the time course analysis (SRTC), three interaction effects were studied: (i) the interaction between response and timepoint between Baseline and Week 12, (ii) the interaction between response and timepoint between Baseline and Week 24, and (iii) the interaction between response and timepoint between Week 12 and Week 24. Interestingly, unlike what was observed in the time course analysis of CC combining all responders into one group versus Placebo, there was an interaction effect of the strength of response and timepoint between Week 12 and Week 24, and not in the other interactions tested, meaning that the differences between Strong and Weak responders kept increasing even though the overall effect of treatment appeared to show no large difference in the TC analysis. CDCA was the only significant difference at the metabolite level, while indicators of bile acid conjugation and the TDCA/CA ratio were still discriminating between Strong and Weak responders in light of the changes that occurred between Week 12 and Week 24.

### CONCLUSION

The CC analysis showed that besides the expected increase in circulating levels of TUDCA and related metabolites, a larger impact on bile acids conjugation could be observed. The PC analysis, including subjects who had to discontinue treatment, showed a similar bile acids profile with no sticking difference. The TC analysis confirmed that the bile acids profile was already set at Week 12 and mostly sustained at Week 24.

The ALSFRS-R slope of CC subjects allowed to study bile acids levels in light of disease progression over the 24 weeks of the study. Only weak correlations could be found in AMX0035-treated subjects, including a weak negative correlation to UDCA. Correlation analysis pointed towards CDCA (negative correlation) and an impact on bile acids conjugation (positive correlations) that primarily occurs in the liver, although these results should be interpreted with caution du e to the weakness of the correlation.

The SR analysis revealed no difference between Strong and Weak responders to the treatment at Baseline or Week 12 At Week 24, the levels of TUDCA, UDCA and GUDCA, as well as the hydrophilic to hydrophobic bile acids ratio were similar in both groups. However, CDCA (a precursor of UDCA via metabolism by the gut microbiota), the TDCA/CA ratio (a suggested indicator of cognitive decline) and indicators of bile acids conjugation to glycine and taurine appeared as the main driving force to differentiate between Strong and Weak responders at the end of treatment.

The SRTC analysis indicated that when discriminating between Strong and Weak responders, differences in bile acids profiles were still evolving between Week 12 and Week 24. Here again, CDCA, TDCA/CA and indicators of bile acid conjugation were the main discriminants.

1.-65. (canceled)

66. A method of treating at least one symptom of Amyotrophic Lateral Sclerosis (ALS) in a subject, the method comprising:
(a) administering to the subject one or more doses of a composition comprising about 1 gram of Taurursodiol (TURSO) and about 3 grams of sodium phenylbutyrate two hours or more after the subject has consumed food, or one hour or more before the subject consumes food,
(b) determining that the subject has (1) a Cₘₐₓ for sodium phenylbutyrate of about 3 to about 425 μg/mL, and/or (2) a Cₘₐₓ for phenylacetate of about 5 to about 50 μg/mL, and
(c) administering to the subject an additional dose of the composition.

67. The method of claim 66, comprising determining that the subject has a Cₘₐₓ for sodium phenylbutyrate of about 90 to about 170 μg/mL.

68. The method of claim 66, comprising determining that the subject has a Cₘₐₓ for phenylacetate of about 10 to about 45 μg/mL.

69. A method of treating at least one symptom of ALS in a subject, the method comprising:
(a) administering to the subject one or more doses of a composition comprising about 1 gram of TURSO and about 3 grams of sodium phenylbutyrate two hours or more after the subject has consumed food, or one hour or more before the subject consumes food,
(b) determining that the subject has (1) an AUC_{0-∞} for sodium phenylbutyrate of about 25 to about 545 μg*h/mL, and/or (2) an AUC_{0-∞} for phenylacetate of about 21 to about 155 μg*h/mL, and
(c) administering to the subject an additional dose of the composition.

70. The method of claim 69, comprising determining that the subject has an AUC_{0-∞} for sodium phenylbutyrate of about 140 to about 300 μg*h/mL.

71. The method of claim 69, comprising determining that the subject has an AUC_{0-∞} for phenylacetate of about 40 to about 80 μg*h/mL.

72. The method of claim 66, wherein step (a) comprises administering the composition once a day or twice a day.

73. The method of claim 66, wherein step (a) comprises administering the composition once a day for about 3 weeks followed by twice a day for about 9 weeks to about 21 weeks.

74. The method of claim 66, wherein step (b) comprises obtaining a blood sample from the subject about one hour after the last dose of the composition or about four hours after the last dose of the composition.

75. A method of treating at least one symptom of ALS in a subject, the method comprising:
(a) administering to the subject one or more doses of a composition comprising about 1 gram of TURSO and about 3 grams of sodium phenylbutyrate two hours or more after the subject has consumed food, or one hour or more before the subject consumes food;
(b) determining that the subject has (1) an AUC₀₋ₗₐₛₜ for sodium phenylbutyrate of about 20 to about 550 μg*h/mL, and/or (2) an AUC₀₋ₗₐₛₜ for phenylacetate of about 20 to about 160 μg*h/mL; and
(c) administering to the subject an additional dose of the composition.

76. The method of claim 75, wherein step (a) comprises administering the composition once a day or twice a day.

77. The method of claim 75, wherein step (a) comprises administering the composition once a day for about 3 weeks followed by twice a day for about 9 weeks to about 21 weeks.

78. The method of claim 75, wherein step (b) comprises-obtaining a blood sample from the subject about one hour after the last dose of the composition or about four hours after the last dose of the composition.

79.-89. (canceled)

90. A method of increasing the plasma concentration of a bile acid in a subject, the method comprising administering to the subject one or more doses of a composition comprising about 1 gram of TURSO and about 3 grams of sodium phenylbutyrate, wherein the bile acid is selected from TURSO, UDCA or GUDCA,
wherein where the bile acid is TURSO, the plasma concentration is about 20 to about 3250 ng/mL, wherein where the bile acid is UDCA, the plasma concentration is about 20 to about 7340 ng/mL, and wherein where the bile acid is GUDCA, the plasma concentration is about 20 to about 5290 ng/mL.

91. The method of claim 90, the method comprising administering the composition once a day or twice a day for about 1 day to about 40 weeks.

92. The method of claim 90, the method comprising administering the composition once a day for about 3 weeks followed by twice a day for about 9 weeks to about 21 weeks.

93. The method of claim 90, the method comprising determining the plasma concentration of the bile acid about one hour after the last dose of the composition or about four hours after the last dose of the composition.

94. The method of claim 66, wherein the composition is administered orally, through a feeding tube, or by bolus injection.

95. The method of claim 66, wherein the composition is a powder formulation.

96. The method of claim 69, wherein step (a) comprises administering the composition once a day or twice a day.

97. The method of claim 69, wherein step (a) comprises administering the composition once a day for about 3 weeks followed by twice a day for about 9 weeks to about 21 weeks.

98. The method of claim 69, wherein step (b) comprises obtaining a blood sample from the subject about one hour after the last dose of the composition or about four hours after the last dose of the composition.

99. The method of claim 75, comprising determining that the subject has an AUC₀₋ₗₐₛₜ for sodium phenylbutyrate of about 140 to about 300 μg*h/mL.

100. The method of claim 75, comprising determining that the subject has an AUC₀₋ₗₐₛₜ for phenylacetate of about 40 to about 80 μg*h/mL.
